(19) United States
(12) Patent Application Publication
(10) Pub. No.: US 2008/0207708 A1
(43) Pub. Date: Aug. 28, 2008

(54) Oxazole and Thiazole Compounds and Their Use in the Treatment of Pge2 Mediated Disorders

(75) Inventors: Rino Antonio Bit, Essex (GB); Adrian Hall, Essex (GB); David Nigel Hurst, Essex (GB); Tiziana Scoccitti, Essex (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXO GROUP LIMITED, GREENFORD MIDDLESEX UNITED KINGDOM (GB)

(21) Appl. No.: 11/912,336

(22) PCT Filed: Apr. 24, 2006
(86) PCT No.: PCT/EP06/03810

§ 371 Date: Mar. 10, 2008

(30) Foreign Application Priority Data
Apr. 26, 2005 (GB) 0508463.7

(51) Int. Cl. A61K 31/426; C07D 277/22; C07D 263/30; A61K 31/421; C07D 413/02; C07D 417/02

(52) U.S. Cl. 514/365; 548/203; 548/235; 514/374

(57) ABSTRACT

Compounds of formula (I) or a pharmaceutically acceptable derivative thereof:

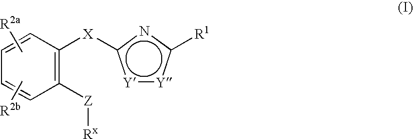

wherein X, Z, Y′, Y″, R¹, R^{2a}, R^{2b}, and R^{x} are as defined in the specification, a process for the preparation of such compounds, pharmaceutical compositions comprising such compounds and the use of such compounds in medicine.

This invention relates to heterocyclic compounds, more specifically thiazole and oxazole compounds, to processes for their preparation, to pharmaceutical compositions containing them and to their use in medicine, in particular their use in the treatment of conditions mediated by the action of PGE₂ at the EP₁ receptor.

Prostaglandin receptors, including the EP₁₋₄, DP, FP IP and TP receptors are the effector proteins for the products (prostaglandins) downstream of COX-1/2 activation (PGE₂, PGD2, PGF2a, PGI2 and thromboxane respectively). The NSAIDS (non-steroidal anti-inflammatory drugs) are indiscriminate cyclooxygenase inhibitors and reduce the levels of these prostaglandins. This in turn reduces the action of the prostaglandins at their respective receptors. In view of the relatively large number of receptors affected, the pharmacology of the NSAIDS is complex.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion.

We have now found a novel group of compounds which bind with high affinity to the EP₁ receptor. These compounds are antagonists of the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S. Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. In The American Physiological Society (1994, 267, R289-R-294), studies suggest that PGE₂-induced hyperthermia in the rat is mediated predominantly through the EP₁ receptor.

The TP (also known as TxA₂) receptor is a prostanoid receptor subtype stimulated by the endogenous mediator thromboxane. Activation of this receptor results in various physiological actions primarily incurred by its platelet aggregatory and smooth muscle constricting effects, thus opposing those of prostacyclin receptor activation.

TP receptors have been identified in human kidneys (G. P. Brown et al, Prostaglandins and other lipid mediators, 1999, 57, 179-188) in the glomerulus and extraglomerular vascular tissue. Activation of TP receptors constricts glomerular capillaries and suppresses glomerular filtration rates (M. D. Breyer et al, Current Opinion in Nephrology and Hypertension, 2000, 9, 23-29), indicating that TP receptor antagonists could be useful for renal dysfunction in glomerulonephritis, diabetes mellitus and sepsis.

Activation of TP receptors induces bronchoconstriction, increase in microvascular permeability, formation of mucosal oedema and mucus secretion, typical characteristic features of bronchial asthma (T. Obata et al, Clinical Review of Allergy, 1994, 12(1), 79-93). TP antagonists have been investigated as potential asthma treatments resulting in, for example, orally active Seratrodast (AA-2414) (S. Terao et al, Yakugaku Zasshi, 1999, 119(5), 377-390). Ramatroban is another TP receptor antagonist currently undergoing phase III clinical trials as an anti-asthmatic compound.

Antagonists at the TP receptor have been shown to have a gastroprotective effect. In rats it has been shown that SQ 33961 and BM 13505 inhibit gastric lesions induced by taurocholate acid, aspirin or indomethacin (E. H. Ogletree et al, Journal of Pharmacology and Experimental Therapeutics, 1992, 263(1), 374-380.

Certain compounds of the present invention also exhibit antagonism at the TP receptor and are therefore indicated to be useful in treating conditions mediated by the action of thromboxane at the TP receptor. Such conditions include those disclosed in WO 2004/039807 (Merck Frosst Canada & Co) which is incorporated herein by reference, and include respiratory diseases e.g. asthma, allergic diseases, male erectile dysfunction, thrombosis, renal disorders and gastric lesions.

WO 96/06822 (7 Mar. 1996), WO 96/11902 (25 Apr. 1996), EP 752421-A1 (8 Jan. 1997), WO 01/19814 (22 Mar. 2001), WO 03/084917 (16 Oct. 2003), WO 03/101959 (11 Dec. 2003), WO 2004/039753 (13 May 2004), WO 2004/083185 (30 Sep. 2004), WO 2005/037786 (28 Apr. 2005), WO 2005/037793 (28 Apr. 2005), WO 2005/037794 (28 Apr. 2005), WO 2005/040128 (6 May 2005), WO 2005/054191 (16 Jun. 2005) and WO2005/108369 (17 Nov. 2005) disdose compounds as being useful in the treatment of prostaglandin mediated diseases.

A. Hall et al, Bioorg. Med. Chem. Lett., 2006, 16, 2666-2671 discloses biaryl heterocyclic EP₁ receptor agonists.

P. Lacombe et al (220th National Meeting of The American Chemical Society, Washington D.C., USA, 20-24 August, 2000) disclosed 2,3-diarylthiophenes as ligands for the human EP₁ prostanoid receptor. Y. Ducharme et al (18^{th} International Symposium on Medicinal Chemistry; Copenhagen, Denmark and Malmo, Sweden; 15^{th}-19^{th} August 2004) disclosed 2,3-diarylthiophenes as EP₁ receptor antagonists. Y. Ducharme et al, Biorg. Med. Chem. Lett., 2005, 15(4) 1155 also discloses 2,3-diarylthiophenes as selective EP₁ receptor antagonists.

Accordingly the present invention provides compounds of formula (I):

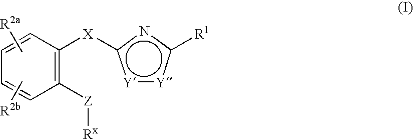

wherein:
either Y′ is CH and Y″ is O or S, or Y′ is O or S and Y″ is CH thus forming an oxazole or a thiazole ring;
X is CR⁷R⁸, O, NR⁴, S, SO, or SO₂, or X is a bond;

### Z is O, S, SO or SO₂;

R^{x} is optionally substituted C₃₋₁₀alkyl, optionally substituted C₃₋₁₀alkenyl, optionally substituted C₃₋₁₀-alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;
R¹ is CO₂H, CQ^{c}Q^{d}CO₂H, tetrazolyl, CH₂tetrazolyl, CONR⁴R⁵, NR⁴CO₂R⁶, NR⁴COR⁶ or 1,2,4-triazol-3-yl optionally substituted on a ring carbon; or R¹ represents imidazolyl or pyrazolyl wherein optionally the imidazole or pyrazole ring is fused to give an optionally substituted bicyclic or tricyclic ring system;
R^{2a} and R^{2b} independently represents hydrogen, halo, CN, SO₂alkyl, SR⁴ or NO₂; or optionally substituted alkyl or optionally substituted alkoxy;
R⁴ is hydrogen or optionally substituted alkyl;
R⁵ is hydrogen or optionally substituted alkyl, optionally substituted aryl, optionally substituted heterocyclyl, optionally substituted SO₂aryl, optionally substituted SO₂alkyl, optionally substituted SO₂heterocyclyl, optionally substituted CQ^{a}Q^{b}aryl, or optionally substituted CQ^{a}Q^{b}heterocyclyl; or
R⁴ and R⁵ together with the nitrogen to which they are attached form a heterocyclic or bicyclic heterocyclic ring;
R⁶ is optionally substituted alkyl or optionally substituted aryl;
R⁷ is hydrogen, fluorine or alkyl;
R⁸ is hydrogen, hydroxy, fluorine or alkyl;
or R⁷ and R⁸ together with the carbon to which they are attached form a cycloalkyl ring, optionally containing up to one heteroatom selected from O, S, NH and N-alkyl; or R⁷ and R⁸ together with the carbon to which they are attached form a carbonyl group; and
Q^{a} and Q^{b} are each independently selected from hydrogen, CH₃ and fluorine;
Q^{c} and Q^{d} are each independently selected from hydrogen and CH₃;
and derivatives thereof;
provided that:
when X is a bond, then R¹ is CQ^{c}Q^{d}CO₂H;
when X is CR⁷R⁸, then R¹ is not CQ^{c}Q^{d}CO₂H;
when R¹ is benzimidazolyl it is unsubstituted on the 1-position; and
when R¹ is benzimidazole optional substituents on the 4 or 7 position are selected from CH₂OH or CO₂H.

Preferably when Y′ or Y″ is O, then R¹ is not CQ^{c}Q^{d}CO₂H.

Preferably the compound of formula (I) is not [2-(5-chloro-2-{[(2,4-difluorophenyl)-methyl]oxy}phenyl)-1,3-oxazol-4-yl]acetic acid (Example 79), [2-(5-chloro-2-{[(2,4,6-trifluorophenyl)methyl]oxy}phenyl)-1,3-oxazol-4-yl]acetic acid (Example 80) or [2-(5-chloro-2-{[(2-chloro-4-fluorophenyl)methyl]oxy}phenyl)-1,3-oxazol-4-yl]acetic acid (Example 81).

Preferably the compound of formula (I) is not 4-[(5-chloro-2-{[(2,3,6-trifluorophenyl)methyl]-oxy}phenyl)methyl]-1,3-thiazole-2-carboxamide (Example 7).

Preferably the compound of formula (I) is not 1,1-Dimethylethyl [2-({5-chloro-2-[(phenylmethyl)oxy]phenyl}methyl)-1,3-oxazol-4-yl]carbamate (Example 161)

Suitably X is CR⁷R⁸, NR⁴, or a bond.

Suitably Z is O.

In one aspect R¹ is not NR⁴CO₂R⁶.

Suitably R¹ is CO₂H, CQ^{c}Q^{d}CO₂H, tetrazolyl, CH₂tetrazolyl, CONR⁴R⁵, NR⁴COR⁶ or 1,2,4-triazol-3-yl optionally substituted on a ring carbon; or R¹ represents imidazolyl or pyrazolyl wherein optionally the imidazole or pyrazole ring is fused to give an optionally substituted bicyclic or tricyclic ring system;

In one aspect R^{2a} is hydrogen.

Suitably R^{2b} is selected from halogen, e.g. Cl or Br, or CF₃.

Preferably R^{2b} is positioned 1,4-relative to the Z substituent and 1,3-relative to the thiazole/oxazole moiety.

In one aspect the compound of formula (I) is a compound of formula (IA):

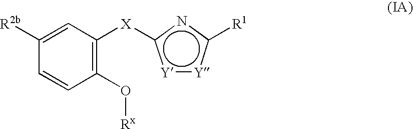

wherein:
either Y′ is CH and Y″ is O or S, or Y′ is O or S and Y″ is CH thus forming an oxazole or a thiazole ring;
X is CR⁷R⁸, or NR⁴, or X is a bond;
R^{x} is optionally substituted C₃₋₁₀alkyl, optionally substituted C₃₋₁₀alkenyl, optionally substituted C₃₋₁₀alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;
R¹ is CO₂H, CQ^{c}Q^{d}CO₂H, tetrazolyl, CH₂tetrazolyl, CONR⁴R⁵, NR⁴COR⁶ or 1,2,4-triazol-3-yl optionally substituted on a ring carbon; or R¹ represents imidazolyl or pyrazolyl wherein optionally the imidazole or pyrazole ring is fused to give an optionally substituted bicyclic or tricyclic ring system;

### R^{2b} is Cl, Br, or CF₃.

R⁴ is hydrogen or optionally substituted alkyl;
R⁵ is hydrogen or optionally substituted alkyl, optionally substituted aryl, optionally substituted heterocyclyl, optionally substituted SO₂aryl, optionally substituted SO₂alkyl, optionally substituted SO₂heterocyclyl, optionally substituted CQ^{a}Q^{b}aryl, or optionally substituted CQ^{a}Q^{b}heterocyclyl; or
R⁴ and R⁵ together with the nitrogen to which they are attached form a heterocyclic or bicyclic heterocyclic ring;
R⁶ is optionally substituted alkyl or optionally substituted aryl;
R⁷ is hydrogen, fluorine or alkyl;
R⁸ is hydrogen, hydroxy, fluorine or alkyl;
or R⁷ and R⁸ together with the carbon to which they are attached form a cycloalkyl ring, optionally containing up to one heteroatom selected from O, S, NH and N-alkyl; or R⁷ and R⁸ together with the carbon to which they are attached form a carbonyl group; and
Q^{a} and Q^{b} are each independently selected from hydrogen, CH₃ and fluorine;
Q^{c} and Q^{d} are each independently selected from hydrogen and CH₃;
and derivatives thereof;
provided that:
when X is a bond, then R¹ is CQ^{c}Q^{d}CO₂H;
when X is CR⁷R⁸, then R¹ is not CQ^{c}Q^{d}CO₂H;
when Y′ or Y″ is O, then R¹ is not CQ^{c}Q^{d}CO₂H;
when R¹ is benzimidazolyl it is unsubstituted on the 1-position; and
when R¹ is benzimidazole optional substituents on the 4 or 7 position are selected from CH₂OH or CO₂H.

In one aspect R¹ is CO₂H, CQ^{c}Q^{d}CO₂H, 1,2,4-triazol-3-yl, 5-methyl-1,2,4-triazolyl, tetrazolyl, CONHR⁵, NHCOR⁶ or imidazolyl wherein optionally the imidazole ring is fused to give an optionally substituted bicyclic or tricyclic ring system.

When R¹ is CQ^{c}Q^{d}CO₂H, suitably it is CH₂CO₂H, C(CH₃)₂CO₂H, or CH(CH₃)CO₂H.

Examples of fused imidazole groups include benzimidazole, imidazo[1,2-a]pyridine, imidazo[4,5-b]pyridine, imidazo[4,5-c]pyridine, imidazo[4,5-b]pyrazine, and 1,5-dihydroimidazo[4,5-f]indazole all of which may be optionally substituted. Suitable optional substituents include halogen e.g. F and C₁, CO₂H, CH₂OH, CH₂CH₂OH, piperazinylalkyl e.g. piperazinylmethyl, pyrrolidinyl, piperidinyl, morpholinyl, CH₂NR^{a}R^{b}, CH₂CH₂NR^{a}R^{b}; wherein R^{a} is hydrogen or methyl and R^{b} is methyl; or R^{a} and R^{b} together with the nitrogen atom to which they are attached form a morpholinyl, piperidinyl, pyrrolidinyl, or piperazinylalkyl, e.g. piperazinylmethyl group.

When R¹ is substituted benzimidazole, preferably it is substituted on the 5 and/or 6 positions.

When R¹ is benzimidazole, in one aspect it is attached to the thiazole or oxazole ring ring through the 2-position carbon atom.

Suitably when R^{x} represents optionally substituted C₃₋₁₀alkyl this group is C₃₋₈alkyl, for example propyl, butyl, pentyl, 2-methylpropyl, 3-methylbutyl, cyclopropylmethylene, cyclobutylmethylene, cyclopentylmethylene, and cyclohexylmethylene. In one aspect the alkyl group is unsubstituted.

When R^{x} represents optionally substituted CQ^{a}Q^{b}-heterocyclyl, optionally substituted CQ^{a}Q^{b}-bicyclic heterocyclyl or optionally substituted CQ^{a}Q^{b}-aryl, suitably R^{x} includes optionally substituted CH₂-heterocyclyl, optionally substituted CH₂-bicyclic heterocyclyl or optionally substituted CH₂-aryl e.g optionally substituted CH₂-phenyl. Optional substituents for CH₂-phenyl include one, two or three substituents each independently selected from Cl, Br and F.

In one aspect R^{x} represents C₃₋₁₀alkyl or optionally substituted CH₂-phenyl.

Suitably R⁴ includes hydrogen and C₁₋₆alkyl. In one aspect R⁴ is hydrogen.

Suitably R⁵ includes hydrogen, C₁₋₆alkyl, phenyl, pyridyl, tetrazolyl, SO₂-phenyl. SO₂C₁₋₆alkyl, optionally substituted SO₂ isoxazole, CH₂pyridyl, and optionally substituted CH₂phenyl.

Suitable substituents for R⁵ when optionally substituted CH₂phenyl include CH₂NR^{c}R^{d} wherein R^{c} methyl and R^{d} is methyl; or R^{c}C and R^{d} together with the nitrogen atom to which they are attached form a morpholinyl, piperidinyl, pyrrolidinyl, or optionally substituted piperazinyl group, e.g. oxopiperazinyl.

Suitably R⁶ includes optionally substituted C₁₋₄alkyl, e.g. methyl, ethyl, isopropyl and benzyl, and optionally substituted phenyl, e.g. PhCH₂OH and PhCH₂piperidine.

Suitably R⁷ includes C₁₋₃alkyl, e.g. CH₃, and hydrogen.

Suitably R⁸ includes C₁₋₃alkyl, e.g. CH₃, and hydrogen.

Suitably Q^{a} is hydrogen.

Suitably Q^{b} is hydrogen.

Compounds of formula (I) include the compounds of examples 1 to 183 and derivatives thereof.

Derivatives of the compound of formula (I) include salts, solvates (including hydrates), solvates (including hydrates) of salts, esters and polymorphs of the compound of formula (I). Derivatives of the compounds of formula (I) include pharmaceutically acceptable derivatives.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The present invention also includes isotopically-labelled compounds, which are identical to the compounds of formula (I), except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ²H, ³H, ¹¹C, ¹⁴C, ¹⁸F, ³⁵S, ¹²³I and ¹²⁵I. Compounds of the present invention and pharmaceutically acceptable derivatives (e.g. salts) of said compounds that contain the aforementioned isotopes and/or other isotopes of other atoms are within the scope of the present invention. Isotopically-labelled compounds of the present invention, for example those into which radioactive isotopes such as ³H and/or ¹⁴C are incorporated, are useful in drug and/or substrate tissue distribution assays. ³H and ¹⁴C are considered useful due to their ease of preparation and detectability. ¹¹C and ¹⁸F isotopes are considered useful in PET (positron emission tomography), and ¹²⁵I isotopes are considered useful in SPECT (single photon emission computerized tomography), all useful in brain imaging. Substitution with heavier isotopes such as 2H can afford certain therapeutic advantages resulting from greater metabolic stability, for example increased in vivo half-life or reduced dosage requirements and, hence, are considered useful in some circumstances. Isotopically labelled compounds of formula (I) of this invention can generally be prepared by carrying out the procedures disclosed in the Schemes and/or in the Examples below, by substituting a readily available isotopically labelled reagent for a non-isotopically labelled reagent.

The following definitions are used herein unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I). In one aspect the term “pharmaceutically acceptable derivative” means any pharmaceutically acceptable salt, solvate or solvate of salt. In an alternative aspect the term “pharmaceutically acceptable derivative” means any pharmaceutically acceptable salt.

It will be appreciated that, for pharmaceutical use, the derivatives referred to above will be pharmaceutically acceptable derivatives, but other derivatives may find use, for example in the preparation of compounds of formula (I) and the pharmaceutically acceptable derivatives thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen” or “halo” are used to represent fluorine, chlorine, bromine or iodine.

The term “alkyl” as a group or part of a group means a straight, branched or cyclic alkyl group or combinations thereof. Unless hereinbefore defined, examples of alkyl include C₁₋₈alkyl, for example methyl, ethyl, n-propyl, iso-propyl, n-butyl, sec-butyl, iso-butyl, t-butyl, pentyl, hexyl, 1,1-dimethylethyl, cyclopropyl, cyclopentyl or cyclohexyl or combinations thereof such as cyclopropylmethylene, cyclohexylmethylene and cyclopentylmethylene.

When used herein the term “cycloalkyl” means a cyclic alkyl group comprising up to eight carbon atoms in a ring.

The term “alkenyl” means linear or branched structures and combinations thereof, of the indicated number of carbon atoms, having at least one carbon-to-carbon double bond, wherein hydrogen may be replaced by an additional carbon to carbon double bond. C₃₋₈alkenyl, for example, includes 2-methyl-2-propenyl and the like.

The term “alkynyl” means linear or branched structures and combinations thereof, of the indicated number of carbon atoms, having at least one carbon-to-carbon triple bond. C₃₋₈alkynyl, for example, includes propynyl and the like.

The term “alkoxy” as a group or as part of a group means a straight, branched or cyclic chain alkoxy group. Unless hereinbefore defined “alkoxy” includes C₁₋₈alkoxy, e.g. methoxy, ethoxy, n-propoxy, iso-propoxy, n-butoxy, sec-butoxy, iso-butoxy, t-butoxy, pentoxy, hexyloxy, cyclopentoxy or cyclohexyloxy. In one aspect “alkoxy” is C₁₋₆alkoxy.

The term “heterocyclyl” as a group or as part of a group means an aromatic or non-aromatic five or six membered ring which contains from 1 to 4 heteroatoms selected from nitrogen, oxygen or sulfur and unsubstituted or substituted by, for example, up to three substituents, preferably one or two substituents. Examples of 5-membered heterocycles include furan, tetrahydrofuran, thiophene, tetrahydrothiophene, pyrrole, pyrroline, pyrrolidine, dioxolane, oxazole, thiazole, imidazole, imidazoline, imidazolidine, pyrazole, pyrazoline, pyrazolidine, isoxazole, isothiazole, oxadiazole, triazole, thiadiazole, and tetrazole. Examples of 6-membered heterocycles include pyran, tetrahydropyran, pyridine, piperidine, dioxane, morpholine, dithiane, thiomorpholine, pyridazine, pyrimidine, pyrazine, piperazine, and triazine.

The term “heterocyclyloxy” as a group or as part of a group refers to an “—O-heterocyclyl” group, wherein the term “heterocyclyl” is as defined above.

The term “aliphatic heterocyclyl” as a group or as part of a group means an aliphatic five or six membered ring which contains 1 or 2 heteroatoms selected from nitrogen, oxygen or sulfur and is unsubstituted or substituted by, for example, up to three substituents, preferably one or two substituents.

The term “aryl” as a group or part of a group means a 5- or 6-membered aromatic ring, for example phenyl, or a 7 to 12 membered bicyclic ring system where at least one of the rings is aromatic, for example naphthyl. An aryl group may be optionally substituted by one or more substituents, for example up to 4, 3 or 2 substituents. Preferably the aryl group is phenyl.

The term “aryloxy” as a group or as part of a group refers to an “—O-aryl” group, wherein the term “aryl” is as defined above.

The term “heteroaryl” as a group or as part of a group means a monocyclic five or six membered aromatic ring, or a fused bicyclic aromatic ring system comprising two of such monocyclic five or six membered aromatic rings. These heteroaryl rings contain one or more heteroatoms selected from nitrogen, oxygen or sulfur, where N-oxides, sulfur oxides and sulfur dioxides are permissible heteroatom substitutions. A heteroaryl group may be optionally substituted by one or more substituents, for example up to 3 or up to 2 substituents. Examples of “heteroaryl” used herein include furyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyrimidinyl, quinolinyl, isoquinolinyl, benzofuryl, benzothienyl, indolyl, and indazolyl.

The term “bicyclic heterocyclyl” when used herein means a fused bicyclic aromatic or non-aromatic bicyclic heterocyclyl ring system comprising up to four, preferably one or two, heteroatoms each selected from oxygen, nitrogen and sulphur. Each ring may have from 4 to 7, preferably 5 or 6, ring atoms. A bicyclic heteroaromatic ring system may include a carbocyclic ring. Examples of bicyclic heterocyclyl groups include quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl or naphthyridinyl.

When the heteroatom nitrogen replaces a carbon atom in an alkyl group, or when nitrogen is present in a heteroaryl, heterocyclyl or bicyclic heterocyclyl group, the nitrogen atom will, where appropriate be substituted by one or two substituents selected from hydrogen and C₁₋₈alkyl, preferably hydrogen and C₁₋₆alkyl, more preferably hydrogen.

Compounds of formula (I) can be prepared as set forth in the following schemes and in the Examples. The following processes form another aspect of the present invention.

Compounds of formula (I) wherein R¹ is CO₂H or CQ^{c}Q^{d}CO₂H, hereinafter referred to as compounds of Formula (Ia), may be prepared by the general route below:

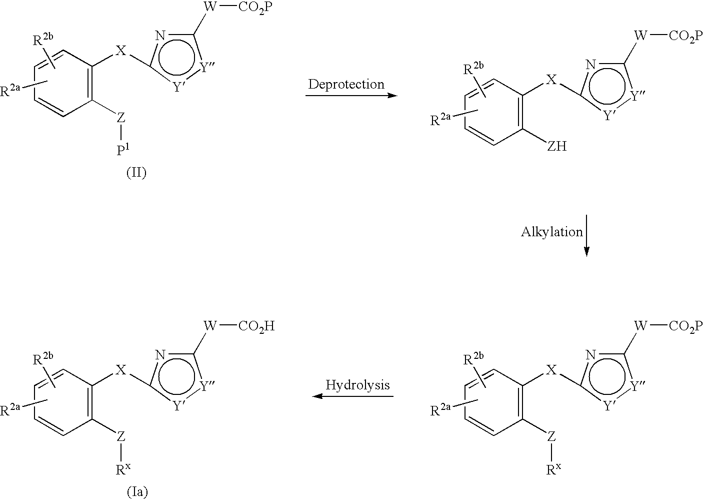

wherein X, Z, R^{2a}, R^{2b} and R^{x} are as defined for compounds of formula (I);
W is CQ^{c}Q^{d} [wherein Q^{c} and Q^{d} are as defined for compounds of formula (I)] or a bond, and P and P¹ are protecting groups.

Compounds of formula (Ia) may be prepared from an intermediate of formula (II) by removal of P¹ followed by reaction with a suitable source of R^{x} wherein R^{x} is as defined for a compound of formula (I). Suitable sources of R^{x} include R^{x}Br. Suitable reaction conditions when the source of R^{x} is R^{x}Br include heating in the presence of a base, e.g. potassium carbonate, in a suitable solvent, e.g. acetone or N,N-dimethylformamide, followed by removal of protecting group P.

Suitable protecting groups will be known to the skilled person. Suitably P is C₁₋₄alkyl or optionally substituted benzyl. Suitable protecting groups when Z is O include C₁₋₄alkyl or benzyl.

Suitable deprotection methods will be known to the skilled person. Conditions for the deprotection of an ester to give the corresponding carboxylic acid are known to those skilled in the art and include heating in the presence of a suitable base, e.g. aqueous sodium hydroxide, in a solvent e.g. an alcohol.

Removal of the protecting group P¹ can be achieved for example through treatment with boron tribromide in a suitable solvent, for example dichloromethane at reduced temperature.

It will be recognised to those skilled in the art that the compounds of formula (I) wherein R¹ is other than CO₂H can be derived from the carboxylic acid (Ia). Compounds wherein R¹ is CONR⁴R⁵, such as amides or acylsulfonamides, can be prepared by activation of the carboxylic acid, for example by forming the acid chloride (for example by reaction of the carboxylic acid with thionyl chloride) followed by reaction with an amine or a sulfonamide respectively. Other derivatives may be accessed by using the Curtius reaction (P.A.S. Smith, Org. React. 3, 337-449 (1946) and J. H. Saunders, R. J. Slocombe, Chem. Rev. 43, 205 (1948)), followed by deprotection of the resulting carbamate and reaction with a carboxylic acid derivative such as an acid chloride. It will be recognised to those skilled in the art that a carboxylic acid group may be converted to a pyrazole, triazole or imidazole group by a sequence of well known functional group transformations such as those described in the Examples. Tetrazoles may be formed from carboxylic acids by converting the carboxylic acid to the primary amide, for example by reaction with thionyl chloride followed by ammonia, followed by dehydration of the amide to the nitrile, for example by heating in phosphorous oxychloride, followed by reaction with azide.

Compounds of formula (I) wherein R¹ is an imidazole moiety fused to give an optionally substituted bicyclic or tricyclic ring system [hereinafter referred to as compounds of formula (Ib)] may be prepared from compounds of formula (III) in accordance with the following scheme:

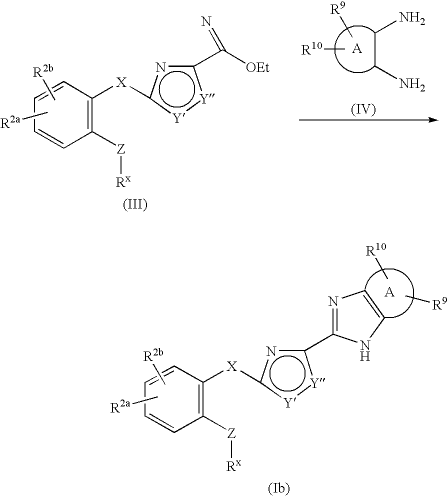

wherein X, Z, R^{2a}, R^{2b}, Y′, Y″ and R^{x} are as defined for compounds of formula (I); A represents e.g. phenyl, pyridine, quinoline, or thiophene, and R⁹ and R¹⁰ each represent hydrogen or a substituent.

Suitable reaction conditions for the preparation of a compound of formula (Ib) include heating the intermediates together in a suitable solvent e.g. ethanol.

Diamines of formula (IV) are commercially available, or may be prepared by known methods.

Compounds of formula (Ib) wherein R¹ is a benzimidazole may also be prepared from the reaction of a diamine of formula (IV) with a compound of formula (I) wherein R¹ is CO₂H.

Compounds of formula (II) when X is CH₂ or a bond, R¹ is CO₂P or CQ^{c}Q^{d}CO₂P, Y′ is S and Y″ is CH may be prepared by known methods. Suitable methods include for example the Hantsch thiazole synthesis comprising the reaction of an α-haloketone with a thioamide as described by the following scheme:

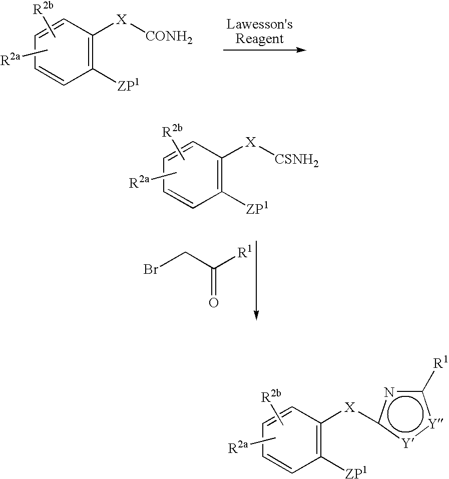

wherein Z, R^{2a}, R^{2b}, P and P¹ are as defined for compounds of formula (I); and P and P¹ are protecting groups as defined above.

Suitable reaction conditions include carrying out the reaction in a suitable solvent such as 1,2-dimethoxyethane in the presence of potassium hydrogen carbonate, trifluoroacetic anhydride and pyridine as described in the examples (Bredekamp, Synth. Comm. 20 (1990) 2235). α-Haloketones are commercially available or can be prepared by known methods. Suitable amide starting materials are commercially available or may be readily prepared from commercially available starting materials by known functional group tranformations.

Compounds of formula (II) when X is CH₂, R¹ is CO₂P or CQ^{c}Q^{d}CO₂P, Y′ is CH and Y″ is S may be prepared by the following scheme:

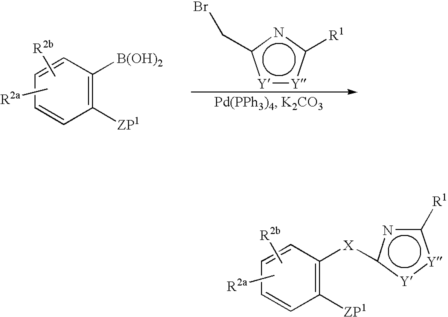

Compounds of formula (II) when X is CH₂ or a bond, R¹ is CO₂P or CQ^{c}Q^{d}CO₂P, Y′ is O and Y″ is CH may be prepared by the following scheme:

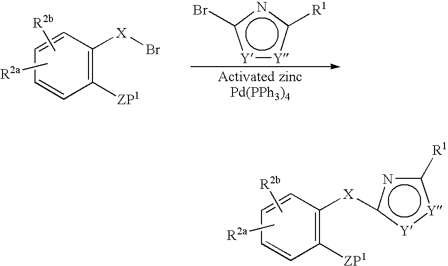

Compounds of formula (III) may be prepared from the corresponding carboxylic acid of formula (Ia) by known methods, for example as described in the Examples. Suitable methods include the reaction of a compound of formula (Ia) with thionyl chloride then ammonia, then phosphorus oxychloride then sodium methoxide in methanol.

Compounds of formula (Ib) wherein R¹ is benzimidazole may be functionalised on the benzimidazole ring using methods described in the Examples and in the scheme below:

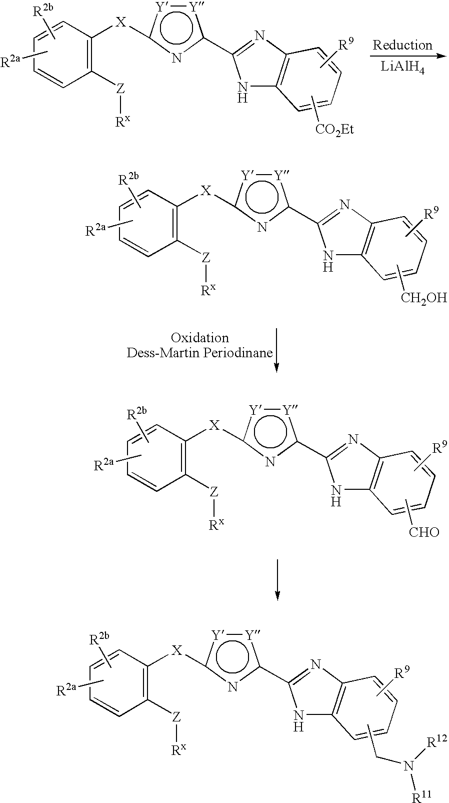

wherein X, Z, R^{2a}, R^{2b}, and R^{x} are as hereinbefore defined for compounds of formula (I) and R¹¹ and R¹² are independently selected from hydrogen, and optionally substituted C₁₋₄alkyl, or R¹¹ and R¹² together with the nitrogen atom to which they are attached form a heterocyclyl ring optionally containing another heteroatom selected from O, NH, NC₁₋₄alkyl, or S.

Accordingly the present invention also provides a process for the preparation of a compound of formula (I) or a derivative thereof:

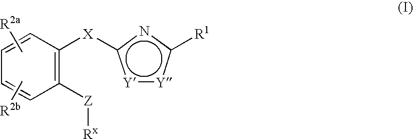

wherein:
either Y′ is CH and Y″ is O or S, or Y′ is O or S and Y″ is CH thus forming an oxazole or a thiazole ring;
X is CR⁷R⁸, O, NR⁴, S, SO, or SO₂, or X is a bond;

### Z is O, S, SO or SO₂;

R^{x} is optionally substituted C₃₋₁₀alkyl, optionally substituted C₃₋₁₀alkenyl, optionally substituted C₃₋₁₀alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;
R¹ is CO₂H, CQ^{c}Q^{d}CO₂H, tetrazolyl, CH₂tetrazolyl, CONR⁴R⁵, NR⁴CO₂R⁶, NR⁴COR⁶ or 1,2,4-triazol-3-yl optionally substituted on a ring carbon; or R¹ represents imidazolyl or pyrazolyl wherein optionally the imidazole or pyrazole ring is fused to give an optionally substituted bicyclic or tricyclic ring system;
R^{2a} and R^{2b} independently represents hydrogen, halo, CN, SO₂alkyl, SR⁴ or NO₂; or optionally substituted alkyl or optionally substituted alkoxy;
R⁴ is hydrogen or optionally substituted alkyl;
R⁵ is hydrogen or optionally substituted alkyl, optionally substituted aryl, optionally substituted heterocyclyl, optionally substituted SO₂aryl, optionally substituted SO₂alkyl, optionally substituted SO₂heterocyclyl, optionally substituted CQ^{a}Q^{b}aryl, or optionally substituted CQ^{a}Q^{b}heterocyclyl; or
R⁴ and R⁵ together with the nitrogen to which they are attached form a heterocyclic or bicyclic heterocyclic ring;
R⁶ is optionally substituted alkyl or optionally substituted aryl;
R⁷ is hydrogen, fluorine or alkyl;
R⁸ is hydrogen, hydroxy, fluorine or alkyl;
or R⁷ and R⁸ together with the carbon to which they are attached form a cycloalkyl ring, optionally containing up to one heteroatom selected from O, S, NH and N-alkyl; or R⁷ and R⁸together with the carbon to which they are attached form a carbonyl group; and
Q^{a} and Q^{b} are each independently selected from hydrogen, CH₃ and fluorine;
Q^{c} and Q^{d} are each independently selected from hydrogen and CH₃;
provided that:
when X is a bond, then R¹ is CQ^{c}Q^{d}CO₂H;
when X is CR⁷R⁸, then R¹ is not CQ^{c}Q^{d}CO₂H;
when R¹ is benzimidazolyl it is unsubstituted on the 1-position; and
when R¹ is benzimidazole optional substituents on the 4 or 7 position are selected from CH₂OH or CO₂H;
comprising:
alkylating a compound:

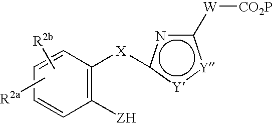

with a source of R^{x};
wherein R^{2a}, R^{2b}, Y′, Y″, R^{x}, Z, X are as defined above for a compound of formula (I), W is CH₂ or a bond, and P is a protecting group; and
in any order:
effecting deprotection; and
if necessary, converting WCO₂H or WCO₂P to another group R¹; and
if necessary forming a derivative thereof.

Certain substituents in any of the reaction intermediates and compounds of formula (I) may be converted to other substituents by conventional methods known to those skilled in the art. Examples of such transformations include the hydrolysis of esters and esterification of carboxylic acids. Such transformations are well known to those skilled in the art and are described in for example, Richard Larock, Comprehensive Organic Transformations, 2nd edition, Wiley-VCH, ISBN 0471-19031-4.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

The compounds of the invention bind to the EP₁ receptor and are antagonists of this receptor. They are therefore considered useful in treating conditions mediated by the action of PGE₂ at EP₁ receptors.

One condition mediated by the action of PGE₂ at EP₁ receptors is pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, multiple sclerosis pain, fibromyalgia, HIV-related neuropathy, post-herpetic neuralgia, trigeminal neuralgia, and pain resulting from physical trauma, amputation, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions mediated by the action of PGE₂ at EP₁ receptors include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) and cyclooxygenase-2 (COX-2) inhibitors, cardiovascular diseases; neurodegenerative diseases and neurodegeneration, neurodegeneration following trauma, tinnitus, dependence on a dependence-inducing agent such as opiods (e.g. morphine), CNS depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence.

Inflammatory conditions include skin conditions (e.g. sunburn, burns, eczema, dermatitis, psoriasis), ophthalmic diseases such as glaucoma, retinitis, retinopathies, uveitis and of acute injury to the eye tissue (e.g. conjunctivitis), inflammatory lung disorders (e.g. asthma, bronchitis, emphysema, allergic rhinitis, respiratory distress syndrome, pigeon fancier's disease, farmer's lung, chronic obstructive pulmonary disease (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendonitis, bursitis, and Sjogren's syndrome.

Immunological diseases include autoimmune diseases, immunological deficiency diseases or organ transplantation. The compounds of formula (I) are also effective in increasing the latency of HIV infection

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendonitis and bursitis.

Cardiovascular diseases include hypertension or myocardiac ischemia; functional or organic venous insufficiency; varicose therapy; haemorrhoids; and shock states associated with a marked drop in arterial pressure (e.g. septic shock).

Neurodegenerative diseases include dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, particularly mesangial proliferative glomerulonephritis and nephritic syndrome.

The compounds of formula (I) are also considered useful for the preparation of a drug with diuretic action.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof.

A proposed daily dosage of compounds of formula (I) or their pharmaceutically acceptable derivatives for the treatment of man is from 0.01 to 80 mg/kg body weight, more particularly 0.01 to 30 mg/kg body weight per day, for example 0.1 to 10 mg/kg body weight per day, which may be administered as a single or divided dose, for example one to four times per day. The dose range for adult human beings is generally from 8 to 4000 mg/day, more particularly from 8 to 2000 mg/day, such as from 20 to 1000 mg/day, for example 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO99/012930); 5-lipoxygenase inhibitors; NSAIDs (non-steroidal anti-inflammatory drugs) such as diclofenac, indomethacin, nabumetone or ibuprofen; leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists; ligands for the α₂δ-subunit of voltage gated calcium channels, such as gabapentin and pregabalin; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands; vanilloid receptor ligand; and purinergic receptor ligands, including antagonists at P2X₃, P2X_{2/3}, P2X₄, P2X₇ or P2X₄/7. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

In addition to activity at the EP₁ receptor, certain compounds of the present invention and pharmaceutically acceptable derivatives thereof exhibit antagonism of the TP receptor and are therefore indicated to be useful in treating conditions mediated by the action of thromboxane at the TP receptor. Conditions mediated by the action of thromboxane at the TP receptor include renal disorders, asthma, or gastric lesions.

In certain situations it is envisaged that the administration of a compound exhibiting antagonism of TP receptors in combination with a compound exhibiting antagonism of EP₁ receptors may be advantageous.

Certain compounds of the invention are selective for EP₁ over EP₃.

No toxicological effects have currently been observed with the compounds of the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

It will be appreciated to those skilled in the art that where compounds are named as hydrochloride salts the stoichiometry of the isolated reaction products is undetermined due to the nature of their preparation. Compounds have therefore been named as hydrochlorides and denoted as xHCl, where x is 0-3 and represents the stoichiometry of said salt.

### Abbreviations

AcOH, acetic acid, Bn (benzyl), Bu, Pr, Me, Et (butyl, propyl, methyl, ethyl), DMSO (dimethyl sulfoxide), DCM/MDC (dichloromethane), DME (ethylene glycol dimethyl ether), DMF (N,N-dimethylformamide), EDAC (N-(3-Dimethylaminopropyl)-N′-ethylcarbodiimide hydrochloride), EDTA (ethylenediaminetetraacetic acid), EtOAc (ethyl acetate), EtOH (ethanol), HOBt (1-Hydroxybenzotriazole), HPLC (High pressure liquid chromatography), IPA (isopropanol), LCMS (Liquid chromatography/Mass spectroscopy), MDAP (Mass Directed Auto Preparation), MeOH (methanol), ML (mother liquor), NMR (Nuclear Magnetic Resonance (spectrum)), NMP (n-methylpyrrolidone), Ph (phenyl), pTSA (para-toluene sulphonic acid), RT/Rt (retention time), SM (starting material), SPE (Solid Phase Extraction—silica cartridge chromatography), TBAF (tetrabutylammonium fluoride), TBME (tertiary butyl methyl ether), THF (tetrahydrofuran), s, d, dd, t, q, m, br (singlet, doublet, double doublet, triplet, quartet, multiplet, broad.).

### Purification of Reaction Products

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the Examples may be carried out by conventional methods such as chromatography and/or recrystallisation using suitable solvents. Chromatographic methods are known to the skilled person and include e.g. column chromatography, flash chromatography, HPLC (high performance liquid chromatography), and MDAP (mass directed autopreparation, also referred to as mass directed LCMS purification). MDAP is described in e.g. W. Goetzinger et al, Int. J. Mass Spectrom., 2004, 238, 153-162.

Flash Master II is an automated chromatography system using commercial prepacked columns. Biotage is a chromatography system using commercial pre-packed silica gel cartridges. The term FLEX (Parallel Flex) when used herein refers to a parallel HPLC purification system.

### LCMS

The following conditions were used for LCMS in the preparation of the examples.
Column: 3.3 cm×4.6 mm ID, 3 μm ABZ+PLUS
Flow Rate: 3 ml/min
Injection Volume: 5 μl
Temp: Room temperature
UV Detection Range: 215 to 330 nm

| Solvents: | A: 0.1% Formic Acid + 10 m Molar Ammonium Acetate. | | |
|---|---|---|---|
| | B: 95% Acetonitrile + 0.05% Formic Acid | | |
| Gradient: | Time | A % | B % |
| | 0.00 | 100 | 0 |
| | 0.70 | 100 | 0 |
| | 4.20 | 0 | 100 |
| | 5.30 | 0 | 100 |
| | 5.50 | 100 | 0 |

All retention times are measured in minutes.

### Preparation of Intermediates

### Ethyl (2-bromo-1,3-thiazol-4-yl)acetate

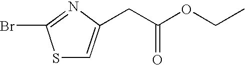

Ethyl 2-amino-4-thiazoleacetate (5 g, 26.8 mmol) was added under nitrogen to a solution of copper(II) bromide (6.77 g, 30 mmol) and t-butyl nitrite (4.79 ml, 40 mmol) in acetonitrile (20 ml) at −20° C. The reaction mixture was slowly warmed to room temperature and stirred for two hours. The solution was then diluted with diethyl ether and washed with 25 ml of 10% hydrochloric acid solution; the aqueous phase was extracted with 20 ml of diethyl ether. The combined organic phases were dried and evaporated to dryness. The residue was purified by flash chromatography with 20% of ethyl acetate in iso-hexane to yield the title compound as a yellow liquid (2.2 g, 32%).

LC/MS Rt=2.33, [MH⁺] 252.

### 4-(4-Morpholinyl)-1,2-benzenediamine

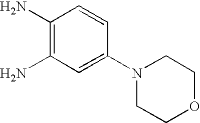

A solution of 5-(4-morpholinyl)-2-nitroaniline (1 g, 4.5 mmol) in DMF (20 ml) with 50 mg of 5% Pd/charcoal was hydrogenated over the weekend. Catalyst was filtered off, the filtrate was evaporated and the residue was chromatographed using 15% of methanol in dichloromethane and triturated with diethyl ether to give a brown solid.

LC/MS Rt=0.39, [MH⁺] 194.2, 195.2

### 2-(3,4-Diaminophenyl)ethanol

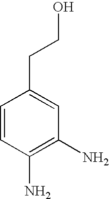

Zinc dust (13.63 g, 208.8 mmol) was added in portions to a stirred solution of 2-(4-amino-3-nitrophenyl)ethanol (3.8 g, 20.88 mmol) in acetic acid (80 ml) with water bath cooling and the mixture stirred for one hour then filtered and evaporated. The residue was re-evaporated with toluene, dissolved in methanol and triethylamine (20 ml) added. The solution was evaporated and purified by flash chromatography on silica eluting with methanol/dichloromethane (8:92) and triturated with ether to give the title compound as an off-white solid which darkened on standing.

LC/MS Rt=0.32 min, [MH⁺] 153.08.

### {5-Chloro-2-[(phenylmethyl)oxy]phenyl}methanol

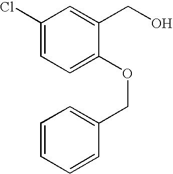

4-Chloro-2-(hydroxymethyl)phenol (5 g, 31 mmol) was dissolved in acetone (30 ml), potassium carbonate (4.78 g, 34 mmol) and benzyl bromide (3.74 ml, 31 mmol) were added. The resulting mixture was refluxed for 2 hours. The reaction was then cooled and the solid was filtered off. The solution was concentrated in vacuo to give the title compound as colorless oil (˜8.2 g).

¹H NMR (CDCl₃)δ: 2.29 (1H, t), 4.68 (2H, d), 5.07 (2H, s), 6.84 (1H, d), 7.6 (1H, d), 7.30-7.39 (6H, m).

### 2-(Bromomethyl)-4-chlorophenyl phenylmethyl ether

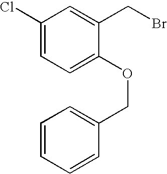

A solution of {5-chloro-2-[(phenylmethyl)oxy]phenyl}methanol (8.2 g, 33 mmol) in dichloromethane (30 ml) was stirred under nitrogen and cooled to −10° C. A solution of phosphorous tribromide (3.12 ml, 33 mmol) in dichloromethane (15 ml) was added slowly at −10° C. and the mixture stirred for 15 minutes at −10° C. The reaction was then allowed to warm to room temperature and was stirred overnight under nitrogen. The reaction mixture was cooled (ice/water bath) and saturated sodium hydrogen carbonate solution was then added slowly and the mixture diluted with dichloromethane and water, brine was added to help the separation of the two phases. The organic phase was separated, washed with water twice then dried (MgSO₄) and evaporated to dryness. The residue was purified by flash chromatography with 5% of ethyl acetate in iso-hexane to yield the title compound as a white solid (8.1 g, 79%).

¹H NMR (CDCl₃)δ: 4.53 (2H, s), 5.14 (2H, s), 6.84 (1H, d, J=8.8 Hz), 7.21 (1H, dd, J=8.8, 2.6 Hz), 7.32-7.47 (6H, m).

### 4-Chloro-2-(chloromethyl)phenyl phenylmethyl ether

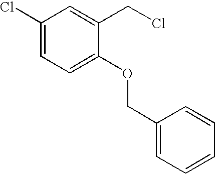

Thionyl chloride (3.37 ml, 50.9 mmol) was added to a solution of {5-chloro-2-[(phenylmethyl)oxy]phenyl}methanol (11.5 g, 46.2 mmol) in DCM (150 ml) and stirred at room temperature for 3 h. Solvent was evaporated and azeotroped with toluene to give 11.5 g of the title compound.

¹H NMR (CDCl₃)δ: 4.63 (2H, s), 5.11 (2H, s), 6.85 (1H, d, J=8.8 Hz), 7.21-7.44 (7H, m)

### 4-Chloro-2-(chloromethyl)phenyl 2-methylpropyl ether

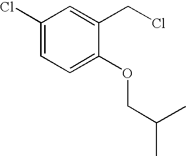

The title compound was prepared in a similar manner to 4-chloro-2-(chloromethyl)phenyl phenylmethyl ether using {5-chloro-2-[(2-methylpropyl)oxy]phenyl}methanol.

¹H NMR (CDCl₃)δ: 1.05 (6H, d, J=6.8 Hz), 2.1-2.16 (1H, m), 3.75 (2H, d, J=6.4 Hz), 4.6 (2H, s), 6.78 (1H, d, J=8.8 Hz), 7.16-7.34 (2H, m)

### {5-Chloro-2-[(2-methylpropyl)oxy]phenyl}methanol

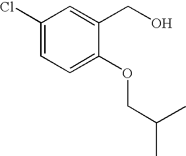

Sodium borohydride (760 mg, 20 mmol) was added to a stirred solution of 5-chloro-2-[(2-methylpropyl)oxy]benzaldehyde (3.8 g, 17.88 mmol) in 50 ml of ethanol and stirred for 1 h. Solvent was evaporated and residue was partitioned between water and ethyl acetate, organic phase was dried and evaporated to give 3.71 g of a pale yellow oil.

¹H NMR (CDCl₃)δ: 1.03 (6H, d, J=6.8 Hz), 2.07-2.14 (1H, m), 2.3-2.4 (1H, bs), 3.75 (2H, d, J=6.4 Hz), 4.66 (2H, s), 6.76 (1H, d, J=8.8 Hz), 7.18 (1H, dd, J=8.8, 2.8 Hz), 7.28 (1H, d, J=2.8 Hz).

### 1-{5-Chloro-2-[(phenylmethyl)oxy]phenyl}ethanone

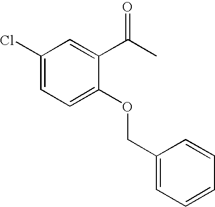

A solution of 1-(5-chloro-2-hydroxyphenyl)ethanone (17.1 g, 100 mmol) in acetone (100 ml) was treated with benzyl bromide (13.08 ml, 110 mmol) and potassium carbonate (16.56 g, 120 mmol). The mixture was stirred and heated to reflux under nitrogen for 2 h. After cooling, the solid was filtered, washed with acetone and the filtrate evaporated. The residue was triturated with iso-hexane and the white solid filtered and dried in vacuo. (24.6 g).

¹H NMR (CDCl₃) δ: 2.59 (3H, s), 5.15 (2H, s), 6.96 (1H, d), 7.35-7.42 (6H, m), 7.71 (1H, d).

### 1-[2-[(Phenylmethyl)oxy]-5-(trifluoromethyl)phenyl]ethanone

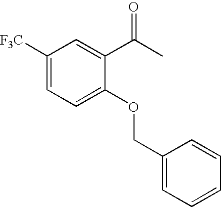

2-[(Phenylmethyl)oxy]-5-(trifluoromethyl)benzoic acid (10 g, 33.8 mmol) was dissolved in dry diethyl ether (100 ml) under nitrogen and 1.4M methyl lithium solution in diethyl ether (72.3 ml, 101.3 mmol) was added dropwise with stirring. A little ice cooling was necessary to stop the ether boiling. The mixture was heated to reflux for 1.5 h, cooled then poured onto a mixture of ice and 2M hydrochloric acid (200 ml). The organic layer was washed with water and 5% sodium bicarbonate solution, dried (MgSO₄) and evaporated. The product was purified by flash chromatography, eluting with 1:1 dichloromethane and iso-hexane to leave a yellow oil (4.1 g).

¹H NMR (CDCl₃) δ: 2.61 (3H, s), 5.23 (2H, s), 7.11 (1H, d), 7.36-7.45 (5H, m), 7.68 (1H, dd), 8.03 (1H, d).

### Ethyl {5-chloro-2-[(phenylmethyl)oxy]phenyl}acetate

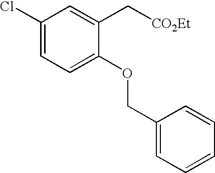

A stirred mixture of 1-{5-chloro-2-[(phenylmethyl)oxy]phenyl}ethanone (15.64 g, 60 mmol), triethyl orthoformate (30 ml) and silver nitrate (21.4 g, 126 mmol) in ethanol (120 ml) under nitrogen was treated with iodine (15.94 g, 63 mmol) and the suspension heated to reflux for 16 h. After cooling, the mixture was filtered and the filtrate evaporated. The residue was dissolved in diethyl ether and the solution washed with water and brine, dried (MgSO₄) and evaporated. The residue was flash chromatographed, eluting with 3-5% ethyl acetate in isohexane to leave a pale yellow solid. (11.58 g).

¹H NMR (CDCl₃) δ: 1.20 (3H, t), 3.62 (2H, s), 4.10 (2H, q), 5.06 (2H, s), 6.83 (1H, d), 7.18 (2H, m), 7.32-7.40 (5H, m).

### Ethyl [2-[(phenylmethyl)oxy]-5-(trifluoromethyl)phenyl]acetate

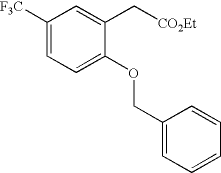

The title compound was prepared in a similar manner to ethyl {5-chloro-2-[(phenylmethyl)oxy]phenyl}acetate using 1-[2-[(phenylmethyl)oxy]-5-(trifluoromethyl)phenyl]ethanone.

¹H NMR (CDCl₃) δ: 1.22 (3H, t), 3.69 (2H, s), 4.12 (2H, q), 5.13 (2H, s), 6.97 (1H, d), 7.32-7.42 (5H, m), 7.47-7.51 (2H, m).

### {5-Chloro-2-[(phenylmethyl)oxy]phenyl}acetic acid

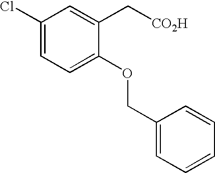

A solution of ethyl {5-chloro-2-[(phenylmethyl)oxy]phenyl}acetate (11.58 g, 38 mmol) in ethanol (60 ml) and water (20 ml) was treated with sodium hydroxide (6.08 g, 152 mmol) and the mixture stirred and heated at 90° C. for 1.5 h. After cooling, the mixture was diluted with water and extracted with diethyl ether. The organic phase was washed with 2M sodium hydroxide solution (50 ml) and the combined aqueous phases acidified (concentrated hydrochloric acid) and extracted with ethyl acetate (2×100 ml). The combined organic phases were washed with water, dried (MgSO₄) and evaporated to an orange oil (6.86 g).

LC/MS Rt=3.30 min, [MH⁺] 277, 279.

### [2-[(Phenylmethyl)oxy]-5-(trifluoromethyl)phenyl]acetic acid

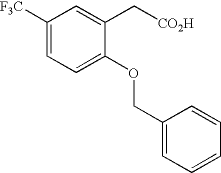

The title compound was prepared in a similar manner to {5-chloro-2-[(phenylmethyl)oxy]phenyl}acetic acid using ethyl [2-[(phenylmethyl)oxy]-5-(trifluoromethyl)phenyl]acetate.

LC/MS Rt=3.37 min.

### {5-Chloro-2-[(2-methylpropyl)oxy]phenyl}acetonitrile

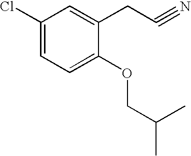

4-Chloro-2-(chloromethyl)phenyl 2-methylpropyl ether (14.7 g, 63.3 mmol) and sodium cyanide (3.41 g, 69.6 mmol) in DMSO (100 ml) were stirred at 60° C. for 1 h. More sodium cyanide (500 mg) was added, stirred at 60° C. for another 40 minutes. Cooled, diluted with water and diethyl ether, the organic phase was washed with water (×3), dried (MgSO₄) and evaporated to give the title compound as yellow oil (13 g).

LC/MS Rt=3.39, [MH⁺] 224.

### {5-Chloro-2-[(phenylmethyl)oxy]phenyl}acetonitrile

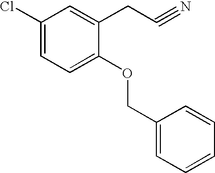

The title compound was prepared in a similar manner to {5-chloro-2-[(2-methylpropyl)oxy]phenyl}acetonitrile using 4-chloro-2-(chloromethyl)phenyl phenylmethyl ether and 1.2 equivalents of sodium cyanide.

¹H NMR (CDCl₃)δ: 3.69 (2H, s), 5.1 (2H, s), 6.87 (1H, d, J=8.8 Hz), 7.24-7.43 (7H, m).

### Methyl {5-chloro-2-[(2-methylpropyl)oxy]phenyl}(oxo)acetate

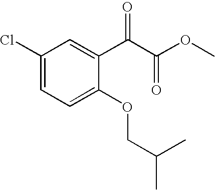

AlCl₃ (2.65 g, 19.9 mmol) was slowly added to a solution of 4-chlorophenyl 2-methylpropyl ether (3 g, 16.6 mmol) and methyl chlorooxoacetate (1.8 ml, 19.9 mmol) in 20 ml of DCM under argon. The reaction mixture was stirred at room temperature for 3 h. Quenched carefully with water and extracted with DCM, the organic phase was dried (MgSO₄) and evaporated. The residue was chromatographed using 20% of DCM in hexane to give the title compound as a yellow solid (1.87 g). LC/MS Rt=3.44 min, [MH⁺] 271.1.

### Methyl {5-chloro-2-[(2-methylpropyl)oxy]phenyl}(difluoro)acetate

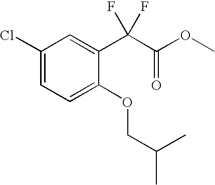

Methyl {5-chloro-2-[(2-methylpropyl)oxy]phenyl}(oxo)acetate (1.67 g, 6.17 mmol) was dissolved in DCM (16 ml) under argon and (diethylamino)sulphur trifluoride (0.97 ml, 7.4 mmol) was added, the mixture was stirred at room temperature overnight. More (diethylamino)sulphur trifluoride (0.3 ml) was added and the mixture was stirred for other 24 h. Quenched with saturated bicarbonate solution and extracted with DCM. The organic phase was dried (MgSO₄) and evaporated to give the title compound as a yellow oil (1.53 g).

¹H NMR (CDCl₃) δ: 1.0 (6H, d, J=6.8 Hz), 1.98-2.05 (1H, m), 3.72 (2H, d, J=6.4 Hz), 3.84 (3H, s), 6.84 (1H, d, J=8.8 Hz), 7.38 (1H, dd, J=8.8, 2.4 Hz), 7.6 (1H, d, J=2.4 Hz).

### 2-{5-Chloro-2-[(2-methylpropyl)oxy]phenyl}-2,2-difluoroacetamide

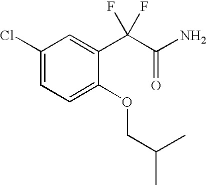

Methyl {5-chloro-2-[(2-methylpropyl)oxy]phenyl}(difluoro)acetate (1.53 g, 5.96 mmol) was treated with 7N ammonia in methanol (6 ml) and stirred at room temperature for 1 hour; solvent was evaporated to give the title compound as a solid (1.45 g).

¹H NMR (CDCl₃) δ: 1.0 (6H, d, J=6.8 Hz), 2.0-2.1 (1H, m), 3.75 (2H, d, J=6.4 Hz), 5.7-5.9 (1H, bs), 6.4-6.6 (1H, bs), 6.85 (1H, d, J=8.8 Hz), 7.38 (1H, dd, J=8.8, 2.4 Hz), 7.63 (1H, d, J=2.8 Hz).

### 2-{5-Chloro-2-[(phenylmethyl)oxy]phenyl}acetamide

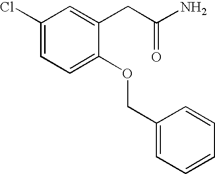

A stirred solution of {5-chloro-2-[(phenylmethyl)oxy]phenyl}acetic acid (6.86 g, 24.79 mmol) in dichloromethane (60 ml) was treated with ammonium 1H-1,2,3-benzotriazol-1-olate (4.15 g, 27.27 mmol), N-(3-dimethylaminopropyl)-N′-ethylcarbodiimide (5.68 g, 29.75 mmol) and N-methylmorpholine (5.45 ml, 49.58 mmol). The mixture was stirred at room temperature for 2 h. The solvent was evaporated and the residue dissolved in ethyl acetate which was washed with 5% sodium bicarbonate solution, 2M hydrochloric acid and water, dried (MgSO₄) and evaporated. The residue was triturated with iso-hexane and diethyl:ether (1:1) and the beige solid filtered and dried in vacuo (4.28 g).

LC/MS Rt=2.88 min, [MH⁺] 276, 278.

### 2-{5-Bromo-2-[(phenylmethyl)oxy]phenyl}acetamide

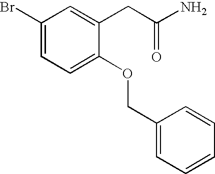

The title compound was prepared in a similar manner to 2-{5-chloro-2-[(phenylmethyl)oxy]phenyl}acetamide using {5-bromo-2-[(phenylmethyl)oxy]phenyl}acetic acid.

¹HNMR (CDCl₃)δ: 3.56 (2H, s), 5.09 (2H, s), 5.2 (1H, bs), 5.6 (1H, bs), 6.85 (1H, d), 7.34-7.40 (7H, m).

### 2-[2-[(Phenylmethyl)oxy]-5-(trifluoromethyl)phenyl]acetamide

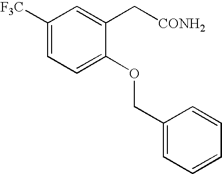

The title compound was prepared in a similar manner to 2-{5-chloro-2-[(phenylmethyl)oxy]phenyl}acetamide using [2-[(phenylmethyl)oxy]-5-(trifluoromethyl)phenyl]acetic acid. LC/MS Rt=2.84 min, [MH⁺] 310.

### 5-Bromo-2-[(phenylmethyl)oxy]benzamide

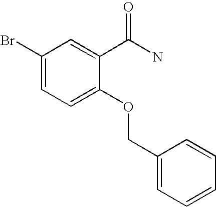

A solution of 5-bromo-2-[(phenylmethyl)oxy]benzoic acid (13.8 g, 45 mmol) and 4-methylmorpholine (4.77 g, 47.2 mmol) in dry tetrahydrofuran (100 ml) was cooled to −12° C. and treated with isobutyl chloroformate (6.44 g, 47.2 mmol). The reaction mixture was stirred at −12° C. for 4 minutes, then 0.880 ammonia (50 ml) was added and the mixture allowed to warm to room temperature. The mixture was diluted with ethyl acetate (50 ml). The organic phase was separated, washed with water and brine, dried and evaporated. The residue was triturated with iso-hexane to give the title compound as a colourless solid 11.1 g, 81%. LC/MS Rt=2.99 min, [MH⁺] 306, 308.

### 2-[5-Chloro-2-(methyloxy)phenyl]acetamide

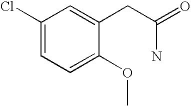

The title compound was prepared in a similar manner to 2-{5-chloro-2-[(phenylmethyl)oxy]phenyl}acetamide using [5-chloro-2-(methyloxy)phenyl]acetic acid.

LC/MS Rt=1.89 min, [MH⁺] 200.

### 2-[5-Bromo-2-(methyloxy)phenyl]acetamide

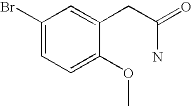

The title compound was prepared in a similar manner to 2-{5-chloro-2-[(phenylmethyl)oxy]phenyl}acetamide using [5-bromo-2-(methyloxy)phenyl]acetic acid.

¹H NMR (CDCl₃) δ: 3.36 (2H, s), 3.75 (3H, s), 6.85 (1H, d, J=8 Hz), 7.25-7.50 (2H, m).

### Methyl 2-{5-chloro-2-[(2-methylpropyl)oxy]phenyl}ethanimidoate hydrochloride

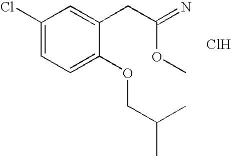

HCL was bubbled through an ice cold solution of {5-chloro-2-[(2-methylpropyl)oxy]phenyl}acetonitrile (13 g, 58.2 mmol) in 80 ml of methanol for ½ h until saturated. The solution was allowed to reach room temperature and left stirring for 2 h. The solvent was evaporated to give a solid which was triturated with Et₂O and filtered off to give 10.4 g of a light pink solid. LC/MS Rt=1.9, [MH⁺] 256.2, 258.2, 259.1.

### Methyl 2-{5-chloro-2-[(phenylmethyl)oxy]phenyl}ethanimidoate hydrochloride

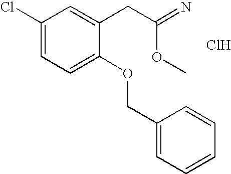

HCL was bubbled through a ice cold solution of {5-chloro-2-[(phenylmethyl) oxy]phenyl}acetonitrile (12.9 g, 50 mmol) in methanol (80 ml) for ½ h until saturated. The solution was allowed to reach room temperature and left stirring for 3 h. LCMS analysis showed the presence of some starting material, the mixture was left in the fridge over the weekend. The solvent was evaporated and the residue was triturated with Et₂O to give the title compound as a white solid. LC/MS Rt=1.97, [MH⁺] 290.1, 292.1.

### 2-{5-Chloro-2-[(phenylmethyl)oxy]phenyl}ethanethioamide

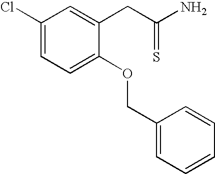

A solution of 2-{5-chloro-2-[(phenylmethyl)oxy]phenyl}acetamide (4.28 g, 15.52 mmol) in 1,2-dimethoxyethane (40 ml) was treated with Lawesson's Reagent (3.14 g, 7.76 mmol). The mixture was stirred at room temperature for 2 h. The solvent was evaporated and the residue dissolved in dichloromethane and washed with 1% sodium hydroxide solution (×2) and brine, dried (MgSO₄) and evaporated. The solid was triturated with diethyl ether, filtered and dried in vacuo (3.43 g). LC/MS Rt=3.32 min, [MH⁺] 292, 294.

### 2-{5-Bromo-2-[(phenylmethyl)oxy]phenyl}ethanethioamide

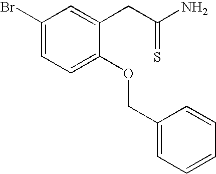

The title compound was prepared in a similar manner to 2-{5-chloro-2-[(phenylmethyl)oxy]phenyl}ethanethioamide using 2-{5-bromo-2-[(phenylmethyl)oxy]phenyl}acetamide.

¹HNMR (CDCl₃)δ: 4.05 (2H, s), 5.10 (2H, s), 6.88 (1H, d), 7.36-7.47 (7H, m).

### 5-Bromo-2-[(phenylmethyl)oxy]benzenecarbothioamide

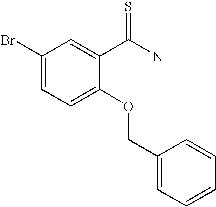

The title compound was prepared in a similar manner to 2-{5-chloro-2-[(phenylmethyl)oxy]phenyl}ethanethioamide using 5-bromo-2-[(phenylmethyl)oxy]benzamide.

¹H NMR (CDCl₃) δ: 5.16 (2H, s), 6.92 (1H, d), 7.39-7.44 (5H, m), 7.53 (1H, d), 7.88-7.90 (1H, br s), 8.75 (1H, s), 8.85-8.90 (1H, br s).

### 2-[2-[(Phenylmethyl)oxy]-5-(trifluoromethyl)phenyl]ethanethioamide

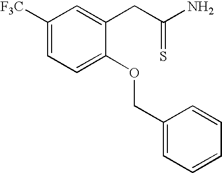

The title compound was prepared in a similar manner to 2-{5-chloro-2-[(phenylmethyl)oxy]phenyl}ethanethioamide using 2-[2-[(phenylmethyl)oxy]-5-(trifluoromethyl)phenyl]acetamide. LC/MS Rt=3.20 min, [MH⁺] 326.

### 2-{5-Chloro-2-[(2-methylpropyl)oxy]phenyl}-2,2-difluoroethanethioamide

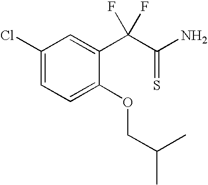

Phosphorus pentasulfide (1.28 g, 2.89 mmol) was added to a solution of 2-{5-chloro-2-[(2-methylpropyl)oxy]phenyl}-2,2-difluoroacetamide (1.6, 5.78 mmol) in 1,2-dimethoxyethane (10 ml), the mixture was stirred at room temperature for 6 h. Diluted with ethyl acetate and washed with a saturated solution of sodium bicarbonate, followed by water. The organic phase was dried (MgSO₄) and evaporated to give a yellow solid (1.54 g).

LC/MS Rt=3.21 min, [MH⁺] 294.1, [MH⁻] 292.1, 294.1

### 2-[5-Chloro-2-(methyloxy)phenyl]ethanethioamide

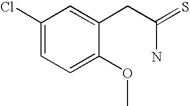

The title compound was prepared in a similar manner to 2-{5-chloro-2-[(phenylmethyl)oxy]phenyl}ethanethioamide using 2-[5-chloro-2-(methyloxy)phenyl]acetamide.

¹H NMR (CDCl₃) δ: 3.85 (3H, s), 4.03 (2H, s), 6.85 (1H, d), 7.23-7.29 (2H, m).

### 2-[5-Bromo-2-(methyloxy)phenyl]ethanethioamide

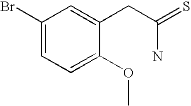

The title compound was prepared in a similar manner to 2-{5-chloro-2-[(phenylmethyl)oxy]phenyl}ethanethioamide using 2-[5-bromo-2-(methyloxy)phenyl]acetamide.

¹H NMR (CDCl₃) δ: 3.85 (3H, s), 4.03 (2H, s), 6.80 (1H, d), 7.38-7-43 (2H, m).

### Methyl 2-({5-chloro-2-[(2-methylpropyl)oxy]phenyl}methyl)-4,5-dihydro-1,3-oxazole-4-carboxylate

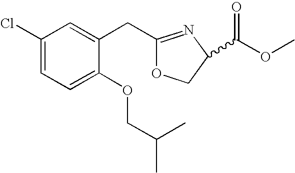

Methyl 2-{5-chloro-2-[(2-methylpropyl)oxy]phenyl}ethanimidoate hydrochloride (10.4 g, 35.6 mmol), diisopropylethylamine (6.2 ml, 35.6 mmol) and DL-serine hydrochloride (5.5 g, 35.6 mmol) in 100 ml of DCM was stirred at room temperature overnight. Washed with water, dried and evaporated; the residue was purified on a Biotage using 30% of ethyl acetate in hexane to give a yellow oil (8.32 g).

LC/MS Rt=3.29, [MH⁺] 326.2, 328.4, 329.2

### Methyl 2-({5-chloro-2-[(phenylmethyl)oxy]phenyl}methyl)-4,5-dihydro-1,3-oxazole-4-carboxylate

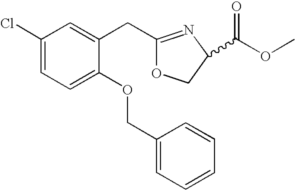

The title compound was prepared in a similar manner to methyl 2-({5-chloro-2-[(2-methylpropyl)oxy]phenyl}methyl)-4,5-dihydro-1,3-oxazole-4-carboxylate.

LC/MS Rt=3.11, [MH⁺] 360, 361, 362, 363

### Methyl 2-({5-chloro-2-[(2-methylpropyl)oxy]phenyl}methyl)-1,3-oxazole-4-carboxylate

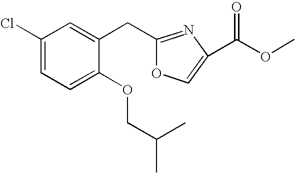

1,8-Diazabicyclo(5.4.0)undec-7-ene (15.3 ml, 102.1 mmol) was added to an ice cold solution of CuBr₂ (22.77 g, 102.1 mmol) and hexamethylene tetramine (14.3 g, 102.1 mmol) in DCM (250 ml); to this mixture methyl 2-({5-chloro-2-[(2-methylpropyl)oxy]phenyl}methyl)-4,5-dihydro-1,3-oxazole-4-carboxylate (8.32 g, 25.5 mmol) was slowly added. The reaction mixture was stirred at room temperature overnight, diluted with diethyl ether and washed (×3) with a 1:1 mixture of aqueous ammonia (0.88) and saturated ammonium chloride solution, followed by H₂O and 2M HCl. The organic phase was dried, evaporated and the residue purified on a Biotage using 20% of ethyl acetate in hexane to give the title compound as a white solid (4.8 g). LC/MS Rt=3.46, [MH⁺] 324.1, 326.1.

### Methyl 2-({5-chloro-2-[(phenylmethyl)oxy]phenyl}methyl)-1,3-oxazole-4-carboxylate

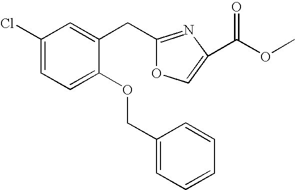

The title compound was prepared in a similar manner to methyl 2-({5-chloro-2-[(2-methylpropyl)oxy]phenyl}methyl)-1,3-oxazole-4-carboxylate.

LC/MS Rt=3.32, [MH⁺] 358.1, 360

### Ethyl {2-[5-chloro-2-(methyloxy)phenyl]-1,3-oxazol-4-yl}acetate

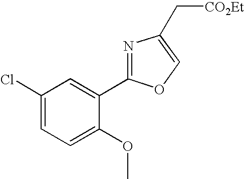

A mixture of ethyl 4-chloroacetoacetate (2.82 g, 17.1 mmol), 5-chloro-2-methoxybenzamide (3.5 g, 18.9 mmol) and pyridine (1.49 g, 18.9 mmol) was heated at 120° C. for 3 hours. After cooling to room temperature the mixture was partitioned between ethyl acetate (25 ml) and water (25 ml). The organic phase was separated, washed with brine, dried and evaporated. Purification of the residue by flash chromatography eluting with 20-50% ethyl acetate in hexane gave the title compound as a yellow solid 1.62 g, 30%.

LC/MS Rt=3.10 min, [MH⁺] 296.

### Ethyl 2-({5-chloro-2-[(phenylmethyl)oxy]phenyl}methyl)-1,3-thiazole-4-carboxylate

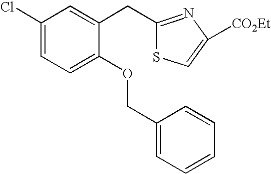

2-{5-Chloro-2-[(phenylmethyl)oxy]phenyl}ethanethioamide (3.43 g, 11.76 mmol) and potassium hydrogen carbonate (9.43 g, 94.3 mmol) were stirred in 1,2-dimethoxyethane (40 ml) under nitrogen for 5 minutes. Ethyl bromopyruvate (4.44 ml, 35.28 mmol) was added and the resulting mixture stirred for 1 minute, then the suspension cooled to 0° C. Trifluoroacetic anhydride (6.64 ml, 47.04 mmol) and pyridine (7.6 ml, 94.3 mmol) were dissolved in 1,2-dimethoxyethane (50 ml) and cooled to 0° C. The solution was added carefully to the original mixture at 0° C. The resulting slurry was stirred and allowed to reach room temperature over 1.5 h. The solvent was evaporated and the residue dissolved in dichloromethane and washed with water (×2), dried (MgSO₄) and evaporated. The residue was purified by flash chromatography, eluting with 20% ethyl acetate in iso-hexane to leave orange oil (3.93 g). LC/MS Rt=3.79 min, [MH⁺] 388, 390.

### Ethyl 2-({5-bromo-2-[(phenylmethyl)oxy]phenyl}methyl)-1,3-thiazole-4-carboxylate

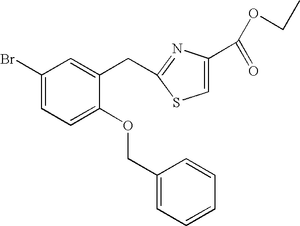

The title compound was prepared in a similar manner to ethyl 2-({5-chloro-2-[(phenylmethyl)oxy]phenyl}methyl)-1,3-thiazole-4-carboxylate using 2-{5-bromo-2-[(phenylmethyl)oxy]phenyl}ethanethioamide. LC/MS Rt=3.84, [MH⁺] 434, 435.

### Ethyl 2-{[2-[(phenylmethyl)oxy]-5-(trifluoromethyl)phenyl]methyl}-1,3-thiazole-4-carboxylate

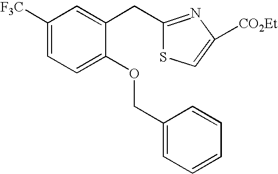

The title compound was prepared in a similar manner to ethyl 2-({5-chloro-2-[(phenylmethyl)oxy]phenyl}methyl)-1,3-thiazole-4-carboxylate using 2-[2-[(phenylmethyl)oxy]-5-(trifluoromethyl)phenyl]ethanethioamide.

LC/MS Rt=3.43 min, [MH⁺] 422.

### Ethyl 2-{[5-chloro-2-[(2-methylpropyl)oxy]phenyl}(difluoro)methyl]-1,3-thiazole-4-carboxylate

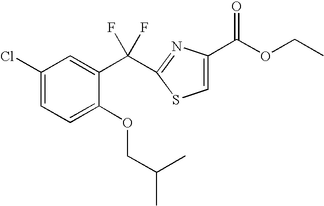

The title compound was prepared in a similar manner to ethyl 2-({5-chloro-2-[(phenylmethyl)oxy]phenyl}methyl)-1,3-thiazole-4-carboxylate using 2-{5-chloro-2-[(2-methylpropyl)oxy]phenyl}-2,2-difluoroethanethioamide.

LC/MS Rt=3.77 min, [MH⁺] 390.1, 392.1

### Ethyl 2-({5-chloro-2-[(phenylmethyl)oxy]phenyl}methyl)-1,3-oxazole-4-carboxylate

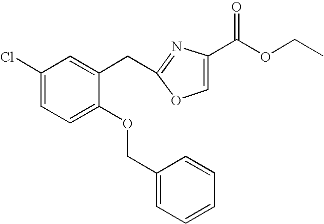

2-(Bromomethyl)-4-chlorophenyl phenylmethyl ether (600 mg, 1.92 mmol) was added dropwise to a suspension of activated zinc dust* (500 mg, 7.7 mmol) in dry THF under a nitrogen atmosphere. As soon as the flask reached room temperature, the solution was filtered under an inert atmosphere and added to a mixture of ethyl 2-bromo-1,3-oxazole-4-carboxylate (333 mg, 1.5 mmol) and Pd(PPh₃)₄ (catalytic) in dry THF under nitrogen. The mixture was stirred at room temperature for 26 h then evaporated and purified using 20% of ethyl acetate in iso-hexane to give the title compound as white solid (240 mg).

LC/MS Rt=3.34, [MH⁺] 372, 374 *Activation of Zinc dust.

A Suspension of 500 mg of zinc in 2 ml of THF containing 1,2-dibromoethane (26 μl, 0.30 mmol) was heated at 65° C. for 1 minute. Cooled to 25° C. and 29 μl of chlorotrimethylsilane (0.23 mmol) was added. The mixture was stirred at 20° C. for 15 minutes before being used.

### [2-({5-Chloro-2-[(phenylmethyl)oxy]phenyl}methyl)-1,3-thiazol-4-yl]methanol

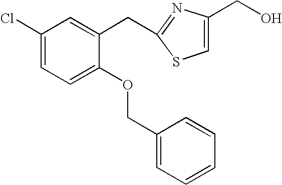

1.0M Lithium aluminium hydride in tetrahydrofuran (0.56 ml, 0.56 mmol) was added, under nitrogen, to a solution of ethyl 2-({5-chloro-2-[(phenylmethyl)oxy]phenyl}methyl)-1,3-thiazole-4-carboxylate (0.2 g, 0.51 mmol) in tetrahydrofuran (4 ml) at −78° C. The reaction mixture was stirred at −78° C. for 1 hour then warmed to room temperature before adding water. The solution was then extracted with dichloromethane, the solvent was dried (MgSO₄) and evaporated to dryness. The residue was purified by flash chromatography eluting with 20% of ethyl acetate in iso-hexane to yield the title compound.

¹H NMR (CDCl₃)δ: 2.09 (1H, bs), 4.32 (2H, s), 4.72 (2H, s), 5.07 (2H, s), 6.86 (1H, d), 7.03 (1H, s), 7.18-7.37 (7H, m).

### (2-{5-Chloro-2-[(phenylmethyl)oxy]phenyl}-1,3-thiazol-4-yl)methanol

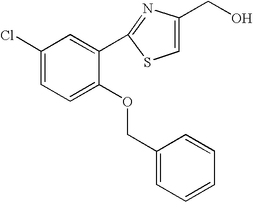

The title compound was prepared in a similar manner to [2-({5-chloro-2-[(phenylmethyl)oxy]phenyl}methyl)-1,3-thiazol-4-yl]methanol using ethyl 2-{5-chloro-2-[(phenylmethyl)oxy]phenyl}-1,3-thiazole-4-carboxylate.

¹H NMR (CDCl₃)δ: 2.28 (1H, t), 4.83 (2H, d), 5.28 (2H, s), 6.99 (1H, d), 7.21 (1H, s), 7.27-7.49 (6H, m), 8.40 (1H, d).

### 2-({5-Chloro-2-[(phenylmethyl)oxy]phenyl}methyl)-1,3-thiazole-4-carbaldehyde

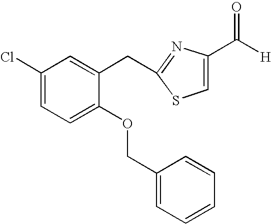

Chromium (VI) oxide (344 mg, 3.47 mmol) was added to a stirred solution of pyridine (0.56 ml, 6.9 mmol) in dichloromethane (5 ml). The mixture was stirred for 15 minutes, then [2-({5-chloro-2-[(phenylmethyl)oxy]phenyl}methyl)-1,3-thiazol-4-yl]methanol (200 mg, 0.58 mmol) in dichloromethane (5 ml) was slowly added. When there was no starting material left the solvent was decanted and the residue washed several times with diethyl ether. The combined organic solutions were washed with 5% sodium hydroxide solution, 5% hydrochloric acid solution, 5% sodium hydrogen carbonate solution and saturated sodium chloride solution. The organic solvent was then dried (MgSO₄) and evaporated to dryness to give the title compound as colourless oil 130 mg.

¹H NMR (CDCl₃)δ: 4.3 (2H, s), 4.99 (2H, s), 6.81 (1H, d), 7.12-7.29 (7H, m), 7.94 (1H, s), 9.9 (1H, s).

### 2-{5-Chloro-2-[(phenylmethyl)oxy]phenyl}-1,3-thiazole-4-carbaldehyde

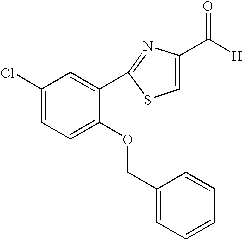

The title compound was prepared in a similar manner to 2-({5-chloro-2-[(phenylmethyl)oxy]phenyl}methyl)-1,3-thiazole-4-carbaldehyde using (2-{5-chloro-2-[(phenylmethyl)oxy]phenyl}-1,3-thiazol-4-yl)methanol.

¹H NMR (CDCl₃)δ: 5.29 (2H, s), 7.04 (1H, d), 7.34-7.48 (6H, m), 8.18 (1H, s), 8.50 (1H, d), 10.1 (1H, s).

### Ethyl (2-{5-bromo-2-[(phenylmethyl)oxy]phenyl}-1,3-thiazol-4-yl)acetate

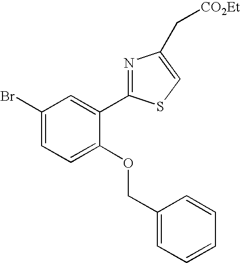

The title compound was prepared in a manner similar to ethyl 2-({5-chloro-2-[(phenylmethyl)oxy]phenyl}methyl)-1,3-thiazole-4-carboxylate using 5-bromo-2-[(phenylmethyl)oxy]benzenecarbothioamide.

LC/MS Rt=4.11 min, [MH⁺] 432, 434.

### 2-[(5-Chloro-2-hydroxyphenyl)methyl]-1,3-thiazole-4-carboxylic acid

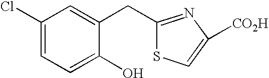

Ethyl 2-({5-chloro-2-[(phenylmethyl)oxy]phenyl}methyl)-1,3-thiazole-4-carboxylate (2.7 g, 6.96 mmol) was dissolved in dimethylformamide (20 ml) and sodium methanethiolate (2.44 g, 34.81 mmol) added. The mixture was heated at 100° C. for 2 h. After cooling, water (100 ml) was added and the mixture extracted with diethyl ether (×2). The aqueous layer was acidified with glacial acetic acid and extracted with ethyl acetate (×2) which was washed with water (×3), dried (MgSO₄) and evaporated to an orange oil 2.01 g.

LC/MS Rt=3.01 min, [MH⁺] 270, 272.

### 2-[(5-Bromo-2-hydroxyphenyl)methyl]-1,3-thiazole-4-carboxylic acid

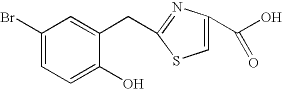

The title compound was prepared in a similar manner to 2-[(5-chloro-2-hydroxyphenyl)methyl]-1,3-thiazole-4-carboxylic acid using ethyl 2-({5-bromo-2-[(phenylmethyl)oxy]phenyl}methyl)-1,3-thiazole-4-carboxylate.

LC/MS Rt=3.09, [MH⁺] 316, 317.

### 2-{[2-Hydroxy-5-(trifluoromethyl)phenyl]methyl}-1,3-thiazole-4-carboxylic acid

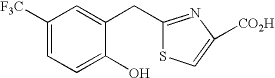

The title compound was prepared in a similar manner to 2-[(5-chloro-2-hydroxyphenyl)methyl]-1,3-thiazole-4-carboxylic acid using ethyl 2-{[2-[(phenylmethyl)oxy]-5-(trifluoromethyl)phenyl]methyl}-1,3-thiazole-4-carboxylate.

### [2-(5-Chloro-2-hydroxyphenyl)-1,3-thiazol-4-yl]acetic acid

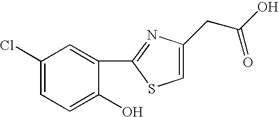

The title compound was prepared in a similar manner to 2-[(5-chloro-2-hydroxyphenyl)methyl]-1,3-thiazole-4-carboxylic acid using ethyl (2-{5-chloro-2-[(phenylmethyl)oxy]phenyl}-1,3-thiazol-4-yl)acetate. LC/MS Rt=3.14, [MH⁺] 270, 272.

### 2-[(5-Chloro-2-hydroxyphenyl)amino]-1,3-thiazole-4-carboxylic acid

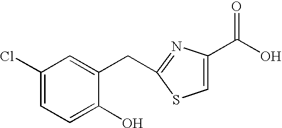

A) The title compound was prepared in a similar manner to 2-[(5-chloro-2-hydroxyphenyl)methyl]-1,3-thiazole-4-carboxylic acid using ethyl 2-{[5-chloro-2-(methyloxy)phenyl]amino}-1,3-thiazole-4-carboxylate and heating the reaction mixture at 60° C. for one day. LC/MS Rt=3.0 min., [MH⁺] 271.

B) Boron tribromide (39.75 g; 15 ml; 158.4 mmol) was added carefully dropwise to a water-bath cooled solution of ethyl 2-{[5-chloro-2-(methyloxy)phenyl]amino}-1,3-thiazole-4-carboxylate (10.0 g, 31.97 mmol) in dichloromethane (250 ml). The resulting mixture was stirred at room temperature for 1 hour, then poured onto ice (˜500 g) and ethyl acetate and water added. The organic layer was dried (MgSO₄) and evaporated to a buff solid 8.65 g.

LC/MS Rt=3.0 min., [MH⁺] 271.

### Ethyl 2-[(5-chloro-2-hydroxyphenyl)methyl]-1,3-thiazole-4-carboxylate

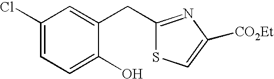

2-[(5-Chloro-2-hydroxyphenyl)methyl]-1,3-thiazole-4-carboxylic acid (2.0 g, 7.42 mmol) was dissolved in ethanol (20 ml) and concentrated sulphuric acid (0.2 ml) added. The solution was heated at reflux for 2 h. The solvent was evaporated and the residue dissolved in diethyl ether and washed with 5% sodium bicarbonate solution and brine, dried (MgSO₄) and evaporated. The residue was purified by flash chromatography, eluting with 30-50% ethyl acetate in iso-hexane to give an orange oil, 983 mg.

LC/MS Rt=3.03 min, [MH⁺] 298, 300.

### Ethyl 2-[(5-bromo-2-hydroxyphenyl)methyl]-1,3-thiazole-4-carboxylate

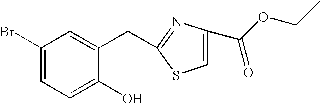

The title compound was prepared in a similar manner to ethyl 2-[(5-chloro-2-hydroxyphenyl)methyl]-1,3-thiazole-4-carboxylate using 2-[(5-bromo-2-hydroxyphenyl)methyl]-1,3-thiazole-4-carboxylic acid.

LC/MS Rt=3.15, [MH⁺] 344, 345.

### Ethyl [2-(5-chloro-2-hydroxyphenyl)-1,3-thiazol-4-yl]acetate

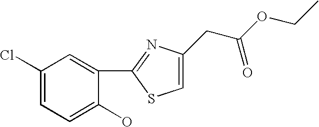

The title compound was prepared in a similar manner to ethyl 2-[(5-chloro-2-hydroxyphenyl)methyl]-1,3-thiazole-4-carboxylate using [2-(5-chloro-2-hydroxyphenyl)-1,3-thiazol-4-yl]acetic acid. LC/MS Rt=3.5, [MH⁺] 298, 301, [MH⁻] 296.

### Ethyl 2-{[2-hydroxy-5-(trifluoromethyl)phenyl]methyl}-1,3-thiazole-4-carboxylate

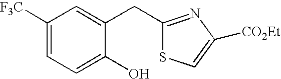

The title compound was prepared in a similar manner to ethyl 2-[(5-chloro-2-hydroxyphenyl)methyl]-1,3-thiazole-4-carboxylate using 2-{[2-hydroxy-5-(trifluoromethyl)phenyl]methyl}-1,3-thiazole-4-carboxylic acid.

LC/MS Rt=2.97 min, [MH⁺] 332.

### Ethyl 2-[(5-chloro-2-hydroxyphenyl)amino]-1,3-thiazole-4-carboxylate

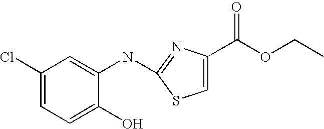

A) The title compound was prepared in a similar manner to ethyl 2-[(5-chloro-2-hydroxyphenyl)methyl]-1,3-thiazole-4-carboxylate using 2-[(5-chloro-2-hydroxyphenyl)amino]-1,3-thiazole-4-carboxylic acid.

LC/MS Rt=3.33 min., [MH⁺] 299, 301.

B) 2-[(5-chloro-2-hydroxyphenyl)amino]-1,3-thiazole-4-carboxylic acid (8.65 g, 31.97 mmol) was dissolved in ethanol (100 ml) and concentrated sulphuric acid (10 ml) added carefully. The solution was heated at 80° C. for 6 hours and left at room temperature overnight. The solvent was evaporated and ethyl acetate and water added. Potassium carbonate was added until the mixture was basic and the organic layer was washed with water, dried (MgSO₄) and evaporated. The residue was triturated with diethyl ether and the solid filtered and dried 6.3 g. LC/MS Rt=2.93 min [MH⁺] 299, 301.

### 4-[(5-Chloro-2-hydroxyphenyl)methyl]-1,3-thiazole-2-carboxamide

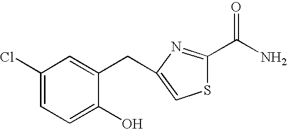

Ethyl 4-({5-chloro-2-[(phenylmethyl)oxy]phenyl}methyl)-1,3-thiazole-2-carboxylate (1 g, 2.57 mmol) in 3.15 ml of hydrogen bromide in acetic acid (48%) was heated at 50° C. for 3 hours. The mixture was then cooled, diluted with water, basified with potassium carbonate and extracted with diethyl ether (3×20 ml). The combined organic phases were dried and evaporated to dryness. The residue was dissolved with ethanol, 3 ml of ammonia were added and the resulting solution was stirred at room temperature overnight. The mixture was then acidified with 2M hydrochloric acid solution and extracted with ethyl acetate (×3). The combined organic phases were dried, filtered and concentrated to yield the title compound (0.6 g, 87%). LC/MS Rt=2.66, [MH⁺] 269, 271.

### Ethyl [2-(5-chloro-2-hydroxyphenyl)-1,3-oxazol-4-yl]acetate

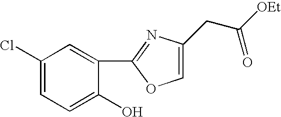

The title compound was prepared from ethyl {2-[5-chloro-2-(methyloxy)phenyl]-1,3-oxazol-4-yl}acetate in a manner similar to that used to prepare ethyl {2-[(5-chloro-2-hydroxyphenyl)methyl]-1,3-thiazol-4-yl}acetate. LC/MS Rt=3.63 min, [MH⁺] 282.

### Ethyl {2-[(5-bromo-2-hydroxyphenyl)methyl]-1,3-thiazol-4-yl}acetate

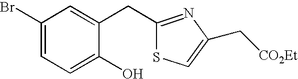

The title compound was prepared from ethyl (2-{[5-bromo-2-(methyloxy)-phenyl]methyl}-1,3-thiazol-4-yl)acetate in a manner similar to that used to prepare ethyl {2-[(5-chloro-2-hydroxyphenyl)methyl]-1,3-thiazol-4-yl}acetate. LC/MS Rt=3.16 min, [MH⁺] 356, 358.

### Ethyl [2-(5-chloro-2-{[(4-fluorophenyl)methyl]oxy}phenyl)-1,3-thiazol-4-yl]acetate

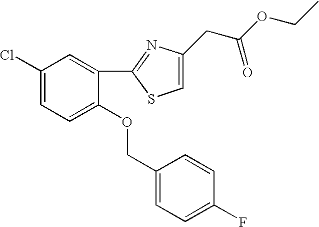

(5-Chloro-2-{[(4-fluorophenyl)methyl]oxy}phenyl)boronic acid (2.96 g, 10.5 mmol), ethyl (2-bromo-1,3-thiazol-4-yl)acetate (2.2 g, 8.8 mmol), potassium carbonate (9.7 g, 70 mmol), and tetrakis(triphenylphosphine)palladium(0) (576 mg, 0.5 mmol) in 1:1 toluene/ethanol (40 ml) were stirred and heated at 90° C. under nitrogen for two hours. After cooling the solvent was evaporated and the residue was diluted with water and extracted with ethyl acetate (3×30 ml), the combined organic phases were dried and evaporated to dryness. The residue was purified by flash chromatography eluting with 15% of ethyl acetate in iso-hexane to yield the title compound as a white solid (2.9 g, 81%). LC/MS Rt=3.77 min, [MH⁺] 406, 409.

### Ethyl 4-({5-chloro-2-[(phenylmethyl)oxy]phenyl}methyl)-1,3-thiazole-2-carboxylate

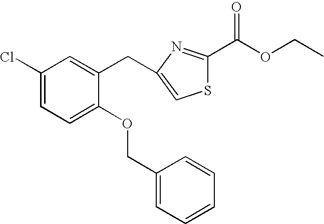

The title compound was prepared in a similar manner to ethyl [2-(5-chloro-2-{[(4-fluorophenyl)methyl]oxy}phenyl)-1,3-thiazol-4-yl]acetate using {5-chloro-2-[(phenylmethyl)oxy]phenyl}boronic acid and ethyl 4-(bromomethyl)-1,3-thiazole-2-carboxylate. LC/MS Rt=3.95 min, [MH⁺] 388.

### Ethyl 4-[(5-chloro-2-{[(4-fluorophenyl)methyl]oxy}phenyl)methyl]-1,3-thiazole-2-carboxylate

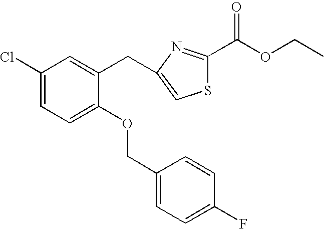

The title compound was prepared in a similar manner to ethyl [2-(5-chloro-2-{[(4-fluorophenyl)methyl]oxy}phenyl)-1,3-thiazol-4-yl]acetate using (5-chloro-2-{[(4-fluorophenyl)methyl]oxy}phenyl)boronic acid and ethyl 4-(bromomethyl)-1,3-thiazole-2-carboxylate. LC/MS Rt=3.95 min, [MH⁺] 406.

### Ethyl (2-{5-chloro-2-[(phenylmethyl)oxy]phenyl}-1,3-thiazol-4-yl)acetate

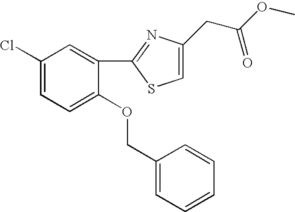

The title compound was prepared in a similar manner to ethyl [2-(5-chloro-2-{[(4-fluorophenyl)methyl]oxy}phenyl)-1,3-thiazol-4-yl]acetate using {5-chloro-2-[(phenylmethyl)oxy]phenyl}boronic acid. LC/MS Rt=4.03 min, [MH⁺] 388.

### Ethyl 2-{5-chloro-2-[(phenylmethyl)oxy]phenyl}-1,3-thiazole-4-carboxylate

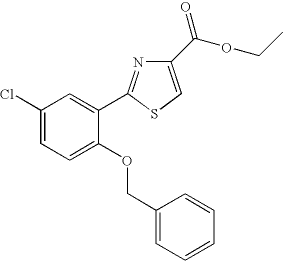

The title compound was prepared in a similar manner to ethyl [2-(5-chloro-2-{[(4-fluorophenyl)methyl]oxy}phenyl)-1,3-thiazol-4-yl]acetate using {5-chloro-2-[(phenylmethyl)oxy]phenyl}boronic acid and ethyl 2-bromo-1,3-thiazole-4-carboxylate.

LC/MS Rt=3.94, [MH⁺] 374, 376.

### Ethyl 2-(2-{5-chloro-2-[(phenylmethyl)oxy]phenyl}-1,3-thiazol-4-yl)propanoate

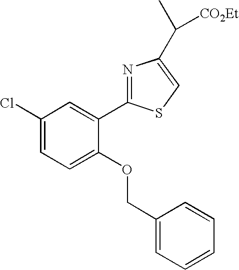

A solution of ethyl (2-{5-chloro-2-[(phenylmethyl)oxy]phenyl}-1,3-thiazol-4-yl)acetate (400 mg, 1.03 mmol) in dry tetrahydrofuran (5 ml) was cooled to −78° C., under N₂ and treated with 2.0M lithium diisopropylamide in heptane/tetrahydrofuran/ethylbenzene (0.5 ml, 1 mmol). The mixture was stirred at −78° C. for 15 mins. Then treated with methyl iodide (142 mg, 1 mmol). After stirring at room temperature for 30mins. the mixture was cooled to −78° C. and treated with a further portion of 2.0M lithium diisopropylamide in heptane/tetrahydrofuran/ethylbenzene (0.5 ml, 1 mmol) and methyl iodide (142 mg, 1 mmol). The mixture was stirred at room temperature for 1 hour then quenched with water (10 ml). The mixture was extracted with diethyl ether (2×10 ml). The combined extracts were dried and evaporated. The residue was purified by flash chromatography, eluting with 8% ethyl actetate in hexane to give the title compound as a colourless oil. 170 mg 42%.

LC/MS Rt=4.16 min, [MH⁺] 416.

### Ethyl 2-(2-{5-bromo-2-[(phenylmethyl)oxy]phenyl}-1,3-thiazol-4-yl)propanoate

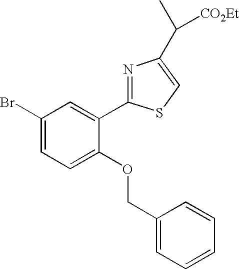

The title compound was prepared from ethyl (2-{5-bromo-2-[(phenylmethyl)oxy]phenyl}-1,3-thiazol-4-yl)acetate in a similar manner to that used to prepare ethyl 2-(2-{5-chloro-2-[(phenylmethyl)oxy]phenyl}-1,3-thiazol-4-yl)propanoate. LC/MS Rt=4.30 min, [MH⁺] 446, 448.

### Ethyl 2-(2-{5-chloro-2-[(phenylmethyl)oxy]phenyl}-1,3-thiazol-4-yl)-2-methylpropanoate

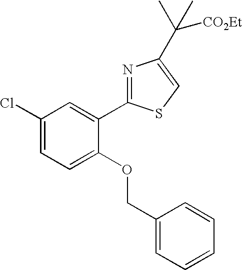

The title compound was prepared in a similar manner to ethyl 2-(2-{5-chloro-2-[(phenylmethyl)oxy]phenyl}-1,3-thiazol-4-yl)propanoate using ethyl 2-(2-{5-chloro-2-[(phenylmethyl)oxy]phenyl}-1,3-thiazol-4-yl)propanoate as the starting material.

LC/MS Rt=4.42 min, [MH⁺] 416.

### Ethyl 2-(2-{5-bromo-2-[(phenylmethyl)oxy]phenyl}-1,3-thiazol-4-yl)-2-methylpropanoate

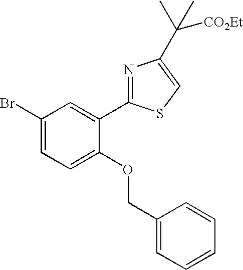

The title compound was prepared in a similar manner to ethyl 2-(2-{5-chloro-2-[(phenylmethyl)oxy]phenyl}-1,3-thiazol-4-yl)propanoate using ethyl 2-(2-{5-bromo-2-[(phenylmethyl)oxy]phenyl}-1,3-thiazol-4-yl)propanoate as the starting material.

LC/MS Rt=4.31 min, [MH⁺] 460, 462.

### General Procedure 1

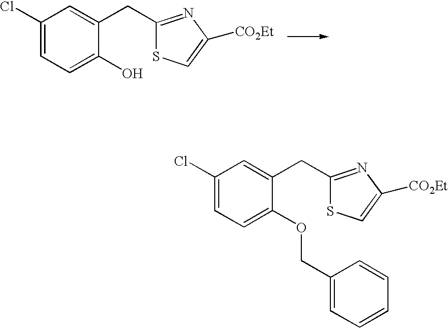

The ethyl 2-[(5-chloro-2-hydroxyphenyl)methyl]-1,3-thiazole-4-carboxylate was dissolved in acetone and potassium carbonate (2.5 to 5 equivalents) was added, followed by the appropriate benzyl bromide (1.1 to 2 equivalents). The reaction was stirred at reflux for between 1 and 18 h. The mixture was filtered and the filtrate evaporated.

The residue was purified by flash chromatography on silica.

The following intermediates were prepared by general procedure 1 from the appropriate starting materials.

| | Name | Data |
|---|---|---|
| 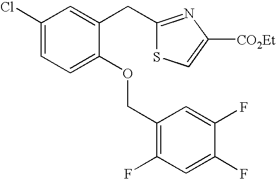 | ethyl 2-[(5-chloro-2-{[(2,4,5-trifluorophenyl)methyl]oxy}phenyl)methyl]-1,3-thiazole-4-carboxylate | LC/MSRt = 3.46 min,[MH⁺] 442,444. |
| 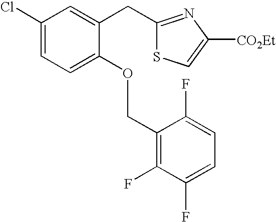 | Ethyl 2-[(5-chloro-2-{[(2,3,6-trifluorophenyl)methyl]oxy}phenyl)methyl]-1,3-thiazole-4-carboxylate | LC/MSRt = 3.41 min,[MH⁺] 442,444. |
| 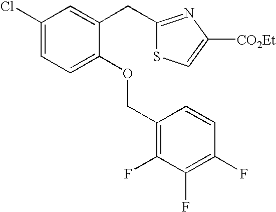 | Ethyl 2-[(5-chloro-2-{[(2,3,4-trifluorophenyl)methyl]oxy}phenyl)methyl]-1,3-thiazole-4-carboxylate | LC/MSRt = 3.47 min,[MH⁺] 442,444. |
| 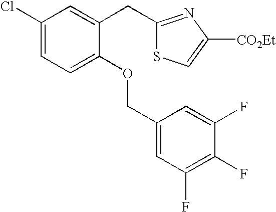 | Ethyl 2-[(5-chloro-2-{[(3,4,5-trifluorophenyl)methyl]oxy}phenyl)methyl]-1,3-thiazole-4-carboxylate | LC/MSRt = 3.45 min,[MH⁺] 442,444. |
| 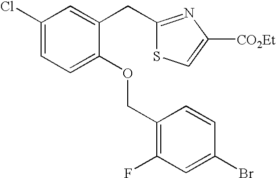 | Ethyl 2-[(2-{[(4-bromo-2-fluorophenyl)methyl]oxy}-5-chlorophenyl)methyl]-1,3-thiazole-4-carboxylate | LC/MSRt = 3.66 min,[MH⁺] 486,488. |
| 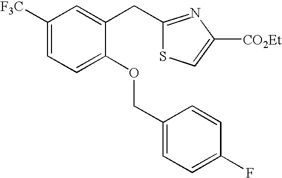 | Ethyl 2-{[2-{[(4-fluorophenyl)methyl]oxy}-5-(trifluoromethyl)phenyl]methyl}-1,3-thiazole-4-carboxylate | LC/MSRt = 3.56 min,[MH⁺] 440. |
| 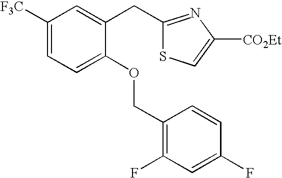 | Ethyl 2-{[2-{[(2,4-difluorophenyl)methyl]oxy}-5-(trifluoromethyl)phenyl]methyl}-1,3-thiazole-4-carboxylate | LC/MSRt = 3.58 min,[MH⁺] 458. |
| 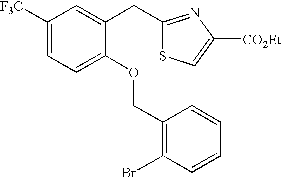 | Ethyl 2-{[2-{[(2-bromophenyl)methyl]oxy}-5-(trifluoromethyl)phenyl]methyl}-1,3-thiazole-4-carboxylate | LC/MSRt = 4.11 min,[MH⁺] 500,502. |
| 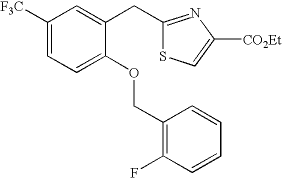 | Ethyl 2-{[2-{[(2-fluorophenyl)methyl]oxy}-5-(trifluoromethyl)phenyl]methyl}-1,3-thiazole-4-carboxylate | LC/MSRt = 3.90 min,[MH⁺] 440. |
| 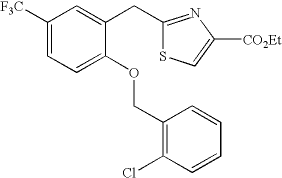 | Ethyl 2-{[2-{[(2-chlorophenyl)methyl]oxy}-5-(trifluoromethyl)phenyl]methyl}-1,3-thiazole-4-carboxylate | LC/MSRt = 4.05 min,[MH⁺] 456,458. |
| 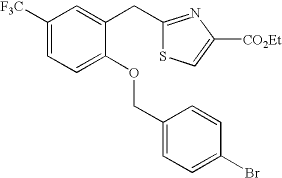 | Ethyl 2-{[2-{[(4-bromophenyl)methyl]oxy}-5-(trifluoromethyl)phenyl]methyl}-1,3-thiazole-4-carboxylate | LC/MSRt = 4.10 min,[MH⁺] 500,502. |
| 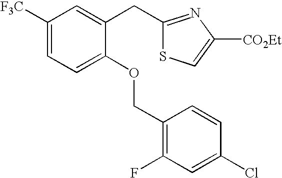 | Ethyl 2-{[2-{[(4-chloro-2-fluorophenyl)methyl]oxy}-5-(trifluoromethyl)phenyl]methyl}-1,3-thiazole-4-carboxylate | LC/MSRt = 4.09 min,[MH⁺] 474,476. |
| 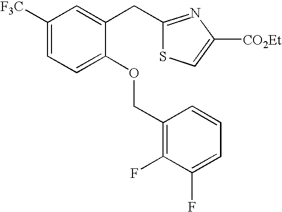 | Ethyl 2-{[2-{[(2,3-difluorophenyl)methyl]oxy}-5-(trifluoromethyl)phenyl]methyl}-1,3-thiazole-4-carboxylate | LC/MSRt = 3.92 min,[MH⁺] 458. |
| 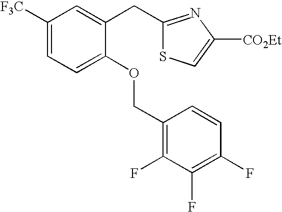 | Ethyl 2-[(5-(trifluoromethyl)-2-{[(2,3,4-trifluorophenyl)methyl]oxy}phenyl)methyl]-1,3-thiazole-4-carboxylate | LC/MSRt = 3.97 min,[MH⁺] 476. |
| 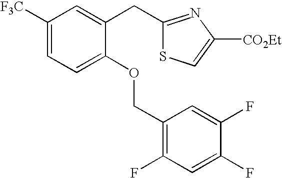 | Ethyl 2-[(5-(trifluoromethyl)-2-{[(2,4,5-trifluorophenyl)methyl]oxy}phenyl)methyl]-1,3-thiazole-4-carboxylate | LC/MSRt = 3.94 min,[MH⁺] 476. |
| 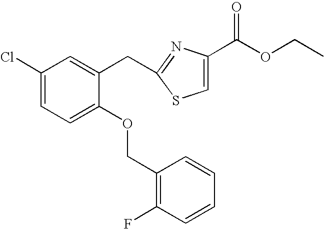 | Ethyl 2-[(5-chloro-2-{[(2-fluorophenyl)methyl]oxy}phenyl)methyl]-1,3-thiazole-4-carboxylate | LC/MSRt = 3.62 min,[MH⁺] 406,408 |
| 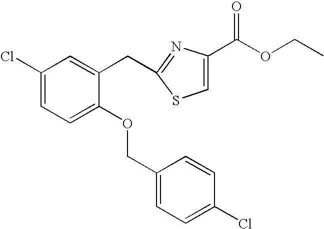 | Ethyl 2-[(5-chloro-2-{[(4-chlorophenyl)methyl]oxy}phenyl)methyl]-1,3-thiazole-4-carboxylate | LC/MSRt = 3.80 min,[MH⁺] 422,425 |
| 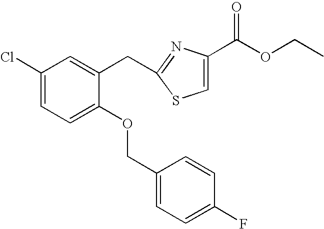 | Ethyl 2-[(5-chloro-2-{[(4-fluorophenyl)methyl]oxy}phenyl)methyl]-1,3-thiazole-4-carboxylate | LC/MSRt = 3.63 min,[MH⁺] 406 |
| 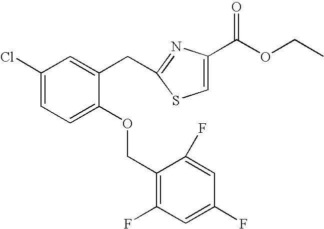 | Ethyl 2-[(5-chloro-2-{[(2,4,6-trifluorophenyl)methyl]oxy}phenyl)methyl]-1,3-thiazole-4-carboxylate | Rt = 3.64 min,[MH+]442, 445 |
| 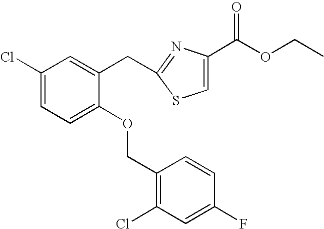 | Ethyl 2-[(5-chloro-2-{[(2-chloro-4-fluorophenyl)methyl]oxy}phenyl)methyl]-1,3-thiazole-4-carboxylate | Rt = 3.88 min[MH+]440, 444 |
| 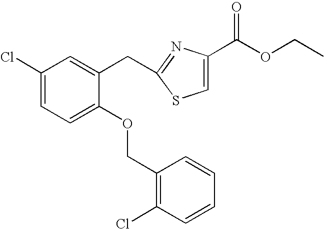 | Ethyl 2-[(5-chloro-2-{[(2-chlorophenyl)methyl]oxy}phenyl)methyl]-1,3-thiazole-4-carboxylate | Rt = 3.86 min.[MH+]422, 426 |
| 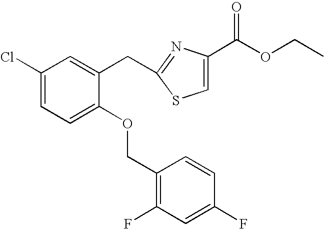 | Ethyl 2-[(5-chloro-2-{[(2,4-difluorophenyl)methyl]oxy}phenyl)methyl]-1,3-thiazole-4-carboxylate | Rt = 3.77 min.[MH+]424, 427 |
| 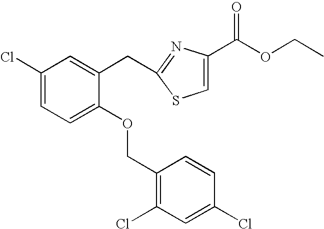 | Ethyl 2-[(5-chloro-2-{[(2,4-dichlorophenyl)methyl]oxy}phenyl)methyl]-1,3-thiazole-4-carboxylate | Rt = 4.16 min.[MH+]458, 460 |
| 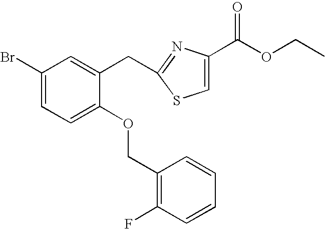 | Ethyl 2-[(5-bromo-2-{[(2-fluorophenyl)methyl]oxy}phenyl)methyl]-1,3-thiazole-4-carboxylate | Rt = 3.9 min.[MH+]452, 453 |
| 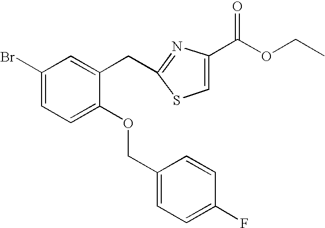 | Ethyl 2-[(5-bromo-2-{[(4-fluorophenyl)methyl]oxy}phenyl)methyl]-1,3-thiazole-4-carboxylate | Rt = 3.9 min.[MH+]452, 453 |
| 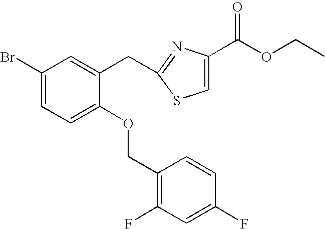 | Ethyl 2-[(5-bromo-2-{[(2,4-difluorophenyl)methyl]oxy}phenyl)methyl]-1,3-thiazole-4-carboxylate | Rt = 3.89 min.[MH+]470, 471 |
| 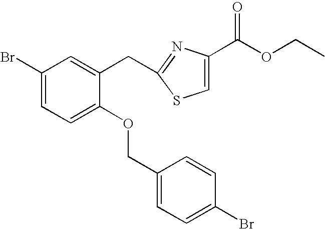 | Ethyl 2-[(5-bromo-2-{[(4-bromophenyl)methyl]oxy}phenyl)methyl]-1,3-thiazole-4-carboxylate | Rt = 4.07 min[MH+]510, 512,514, 515 |
| 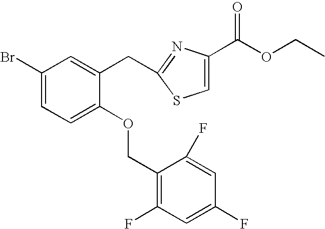 | Ethyl 2-[(5-bromo-2-{[(2,4,6-trifluorophenyl)methyl]oxy}phenyl)methyl]-1,3-thiazole-4-carboxylate | Rt = 3.83 min[MH+]488, 489 |
| 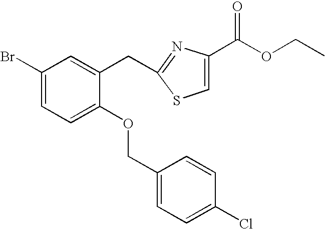 | Ethyl 2-[(5-bromo-2-{[(4-chlorophenyl)methyl]oxy}phenyl)methyl]-1,3-thiazole-4-carboxylate | Rt = 3.88 min[MH+]468, 470 |
| 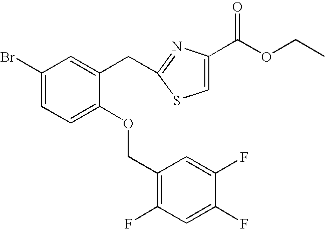 | Ethyl 2-[(5-bromo-2-{[(2,4,5-trifluorophenyl)methyl]oxy}phenyl)methyl]-1,3-thiazole-4-carboxylate | Rt = 3.89 min.[MH+]488, 489 |
| 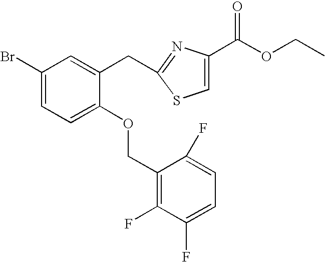 | Ethyl 2-[(5-bromo-2-{[(2,3,6-trifluorophenyl)methyl]oxy}phenyl)methyl]-1,3-thiazole-4-carboxylate | Rt = 3.83 min[MH+]488, 489 |
| 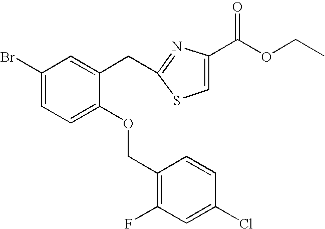 | Ethyl 2-[(5-bromo-2-{[(4-chloro-2-fluorophenyl)methyl]oxy}phenyl)methyl]-1,3-thiazole-4-carboxylate | Rt = 4.05 min[MH+]486, 488 |
| 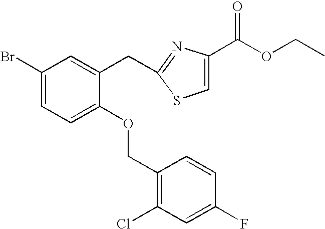 | Ethyl 2-[(5-bromo-2-{[(2-chloro-4-fluorophenyl)methyl]oxy}phenyl)methyl]-1,3-thiazole-4-carboxylate | Rt = 4.07 min[MH+]486, 488 |
| 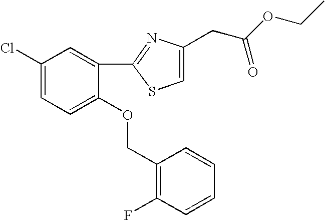 | Ethyl {2-(5-chloro-2-{[(2-fluorophenyl)methyl]oxy}phenyl)-1,3-thiazol-4-yl]acetate | Rt = 3.92 min[MH+]406, 408 |
| 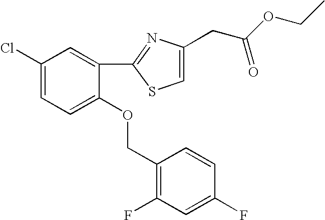 | Ethyl [2-(5-chloro-2-{[(2,4-difluorophenyl)methyl]oxy}phenyl)-1,3-thiazol-4-yl]acetate | Rt = 3.99 min[MH+]424, 426 |
| 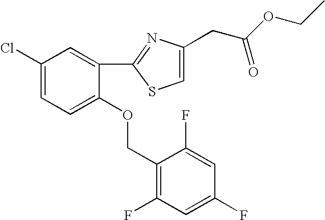 | Ethyl [2-(5-chloro-2-{[(2,4,6-trifluorophenyl)methyl]oxy}phenyl)-1,3-thiazol-4-yl]acetate | Rt = 3.99 min[MH+]442, 444 |
| 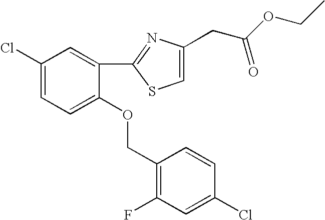 | Ethyl [2-(5-chloro-2-{[(4-chloro-2-fluorophenyl)methyl]oxy}phenyl)-1,3-thiazol-4-yl]acetate | Rt = 4.0 min.[MH+]440, 444 |
| 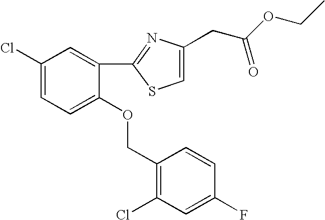 | Ethyl [2-(5-chloro-2-{[(2-chloro-4-fluorophenyl)methyl]oxy}phenyl)-1,3-thiazol-4-yl]acetate | Rt = 4.22 min[MH+]440, 443 |
| 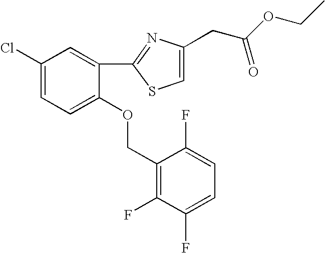 | Ethyl [2-(5-chloro-2-{[(2,3,6-trifluorophenyl)methyl]oxy}phenyl)-1,3-thiazol-4-yl]acetate | Rt = 3.9 min[MH+]442, 444 |
| 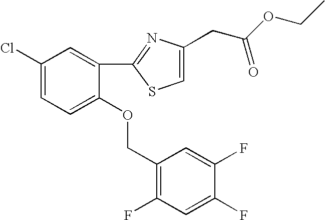 | Ethyl [2-(5-chloro-2-{[(2,4,5-trifluorophenyl)methyl]oxy}phenyl)-1,3-thiazol-4-yl]acetate | Rt = 3.4 min[MH+]442, 444 |
| 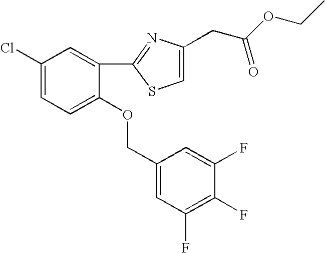 | Ethyl [2-(5-chloro-2-{[(3,4,5-trifluorophenyl)methyl]oxy}phenyl)-1,3-thiazol-4-yl]acetate | Rt = 4.11 min[MH+]442, 444 |
| 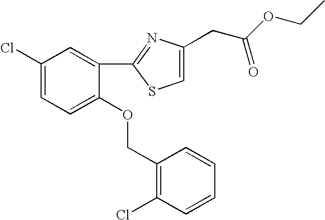 | Ethyl [2-(5-chloro-2-{[(2-chlorophenyl)methyl]oxy}phenyl)-1,3-thiazol-4-yl]acetate | Rt = 4.0 min[MH+]422, 425 |
| 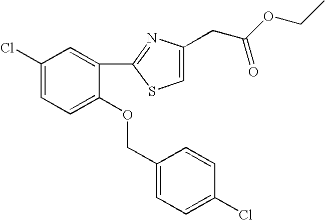 | Ethyl [2-(5-chloro-2-{[(4-chlorophenyl)methyl]oxy}phenyl)-1,3-thiazol-4-yl]acetate | Rt = 3.6 min[MH+]422, 426 |
| 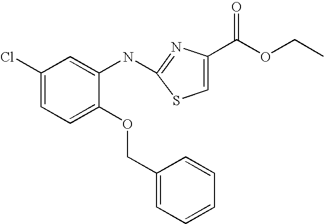 | Ethyl 2-({5-chloro-2-[(phenylmethyl)oxy]phenyl}amino)-1,3-thiazole-4-carboxylate | Rt = 3.83 min[MH+]389, 391 |
| 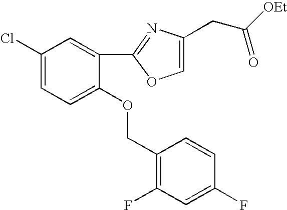 | Ethyl [2-(5-chloro-2-{[(2,4-difluorophenyl)methyl]oxy}phenyl)-1,3-oxazol-4-yl]acetate | Rt = 3.71 min[MH+]408. |
| 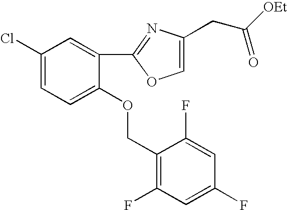 | Ethyl [2-(5-chloro-2-{[(2,4,6-trifluorophenyl)methyl]oxy}phenyl)-1,3-oxazol-4-yl]acetate | Rt = 3.61 min[MH+]426. |
| 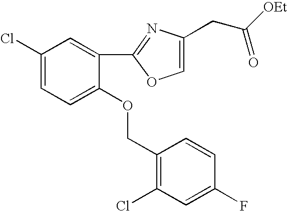 | Ethyl [2-(5-chloro-2-{[(2-chloro-4-fluorophenyl)methyl]oxy}phenyl)-1,3-oxazol-4-yl]acetate | Rt = 3.92 min[MH+]424. |

The following examples were prepared by general procedure 1 from the appropriate intermediates.

| Example | | Name | Data |
|---|---|---|---|
| 1 | 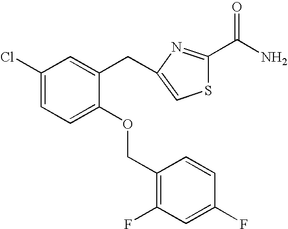 | 4-[(5-chloro-2-{[(2,4-difluorophenyl)methyl]oxy}phenyl)methyl]-1,3-thiazole-2-carboxamide | Rt = 3.60 min[MH+]395, 397 |
| 2 | 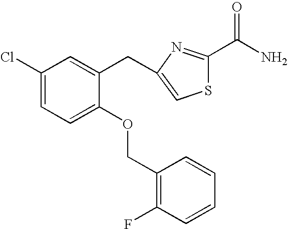 | 4-[(5-chloro-2-{[(2-fluorophenyl)methyl]oxy}phenyl)methyl]-1,3-thiazole-2-carboxamide | Rt = 3.57 min[MH+]377, 379 |
| 3 | 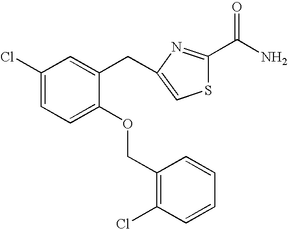 | 4-[(5-chloro-2-{[(2-chlorophenyl)methyl]oxy}phenyl)methyl]-1,3-thiazole-2-carboxamide | Rt = 3.77 min[MH+]393, 397 |
| 4 | 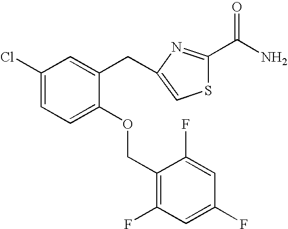 | 4-[(5-chloro-2-{[(2,4,6-trifluorophenyl)methyl]oxy}phenyl)methyl]-1,3-thiazole-2-carboxamide | Rt = 3.57 min[MH+]413, 415 |
| 5 | 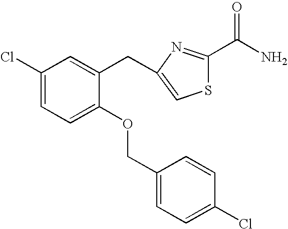 | 4-[(5-chloro-2-{[(4-chlorophenyl)methyl]oxy}phenyl)methyl]-1,3-thiazole-2-carboxamide | Rt = 3.73 min[MH⁺]393 |
| 6 | 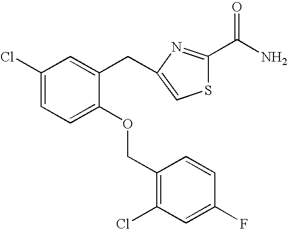 | 4-[(5-chloro-2-{[(2-chloro-4-fluorophenyl)methyl]oxy}phenyl)methyl]-1,3-thiazole-2-carboxamide | Rt = 3.78 min[MH⁺]411, 415 |
| 7 | 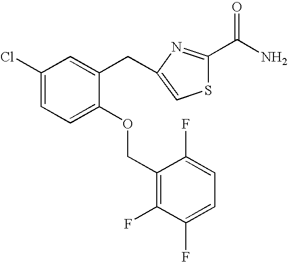 | 4-[(5-chloro-2-{[(2,3,6-trifluorophenyl)methyl]oxy}phenyl)methyl]-1,3-thiazole-2-carboxamide | Rt = 3.56 min[MH⁺]413, 415 |
| 8 | 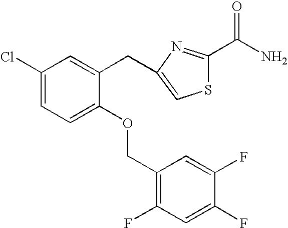 | 4-[(5-chloro-2-{[(2,4,5-trifluorophenyl)methyl]oxy}phenyl)methyl]-1,3-thiazole-2-carboxamide | Rt = 3.63 min[MH⁺]413, 415 |

### General Procedure 2:

### Ethyl 2-({5-chloro-2-[(2-methylpropyl)oxy]phenyl}methyl)-1,3-thiazole-4-carboxylate

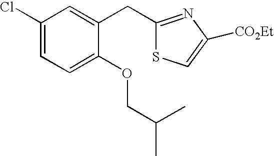

Ethyl 2-[(5-chloro-2-hydroxyphenyl)methyl]-1,3-thiazole-4-carboxylate (98 mg, 0.329 mmol) was dissolved in dimethylformamide (2 ml) and potassium carbonate (227 mg, 1.65 mmol) added, followed by 1-iodo-2-methylpropane (89 mg, 0.395 mmol). The reaction was stirred at 90° C. for 6 h. The mixture was filtered and the filtrate evaporated. The residue was purified by flash chromatography, eluting with 20% ethyl acetate in iso-hexane (53 mg).

LC/MS Rt=3.44 min, [MH⁺] 354, 356.

The following intermediates were prepared by general procedure 2 from the appropriate starting materials.

| | Name | Data |
|---|---|---|
| 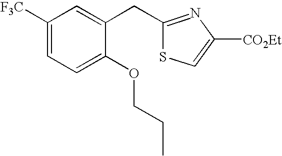 | Ethyl 2-{[2-(propyloxy)-5-(trifluoromethyl)phenyl]methyl}-1,3-thiazole-4-carboxylate | LC/MSRt = 3.77 min,[MH⁺] 374. |
| 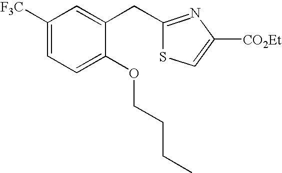 | Ethyl 2-{[2-(butyloxy)-5-(trifluoromethyl)phenyl]methyl}-1,3-thiazole-4-carboxylate | LC/MSRt = 3.93 min,[MH⁺] 388. |
| 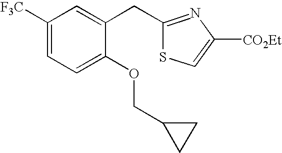 | Ethyl 2-{[2-[(cyclopropylmethyl)-oxy]-5-(trifluoromethyl)phenyl]methyl}-1,3-thiazole-4-carboxylate | LC/MSRt = 3.77 min,[MH⁺] 386. |
| 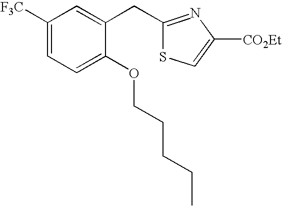 | Ethyl 2-{[2-(pentyloxy)-5-(trifluoromethyl)phenyl]methyl}-1,3-thiazole-4-carboxylate | LC/MSRt = 4.09 min,[MH⁺] 402. |
| 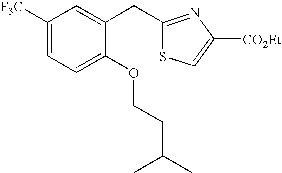 | Ethyl 2-{[2-[(3-methylbutyl)oxy]-5-(trifluoromethyl)phenyl]methyl}-1,3-thiazole-4-carboxylate | LC/MSRt = 4.05 min,[MH⁺] 402. |
| 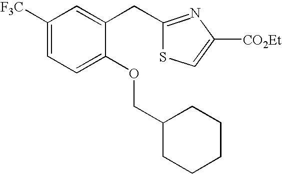 | Ethyl 2-{[2-[(cyclohexylmethyl)oxy]-5-(trifluoromethyl)phenyl]methyl}-1,3-thiazole-4-carboxylate | LC/MSRt = 4.26 min,[MH⁺] 428. |
| 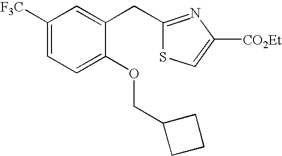 | Ethyl 2-{[2-[(cyclobutylmethyl)oxy]-5-(trifluoromethyl)phenyl]methyl}-1,3-thiazole-4-carboxylate | LC/MSRt = 3.99 min,[MH⁺] 400. |
| 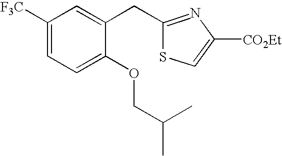 | Ethyl 2-{[2-[(2-methylpropyl)oxy]-5-(trifluoromethyl)phenyl]methyl}-1,3-thiazole-4-carboxylate | LC/MSRt = 3.92 min,[MH⁺] 388. |

### Ethyl 2-{5-chloro-2-[(cyclopentylmethyl)oxy]phenyl}methyl)-1,3-thiazole-4-carboxylate

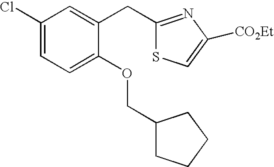

Ethyl 2-[(5-chloro-2-hydroxyphenyl)methyl]-1,3-thiazole-4-carboxylate (130 mg, 0.437 mmol) was dissolved in tetrahydrofuran (2 ml) under nitrogen. Triphenylphosphine (115 mg, 0.478 mmol), diethyl azodicarboxylate (91 μl, 0.478 mmol) and cyclopentanemethanol (47 μl, 0.437 mmol) were added and the mixture stirred at room temperature for 16 h. The reaction was incomplete, so powdered 4A sieve (50 mg) was added, followed by triphenylphosphine (115 mg, 0.478 mmol), diethyl azodicarboxylate (91 μl, 0.478 mmol) and cyclopentanemethanol (47 μl, 0.437 mmol). The reaction was stirred for a further 3 h. The solvent was evaporated and the residue purified by flash chromatography, eluting with 10% ethyl acetate in iso-hexane (62 mg). LC/MS Rt=4.00 min, [MH⁺] 326, 328.

### Ethyl 2-{[2-[(cyclopentylmethyl)oxy]-5-(trifluoromethyl)phenyl]methyl}-1,3-thiazole-4-carboxylate

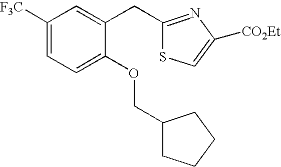

The title compound was prepared in a similar manner to ethyl 2-({5-chloro-2-[(cyclopentylmethyl)oxy]phenyl}methyl)-1,3-thiazole-4-carboxylate using ethyl 2-{[2-hydroxy-5-(trifluoromethyl)phenyl]methyl}-1,3-thiazole-4-carboxylate and cyclopentanemethanol. LC/MS Rt=4.15 min, [MH⁺] 414.

### General Procedure 3

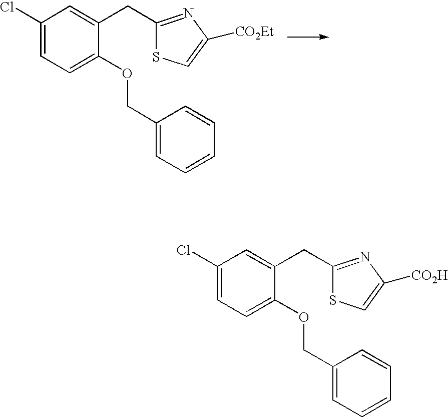

Ethyl 2-({5-chloro-2-[(phenylmethyl)oxy]phenyl}methyl)-1,3-thiazole-4-carboxylate was dissolved in ethanol and an excess of sodium hydroxide added [either 2M sodium hydroxide solution or solid NaOH followed by H₂O]. The solution was stirred at room temperature or warmed to 60° C. overnight.

The solvent was evaporated and the residue diluted with water, extracted with 1:1 diethyl ether and iso-hexane and the aqueous layer acidified with 1M hydrochloric acid or acetic acid. The mixture was extracted with either dichloromethane, diethyl ether or ethyl acetate; the organic phases were washed with water, dried and evaporated.

The following examples were prepared by general procedure 3 from the appropriate intermediates

| Example | | Name | Data |
|---|---|---|---|
| 9 | 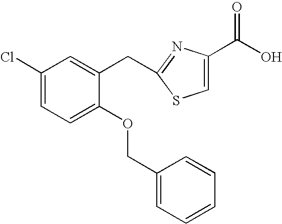 | 2-({5-chloro-2-[(phenylmethyl)oxy]phenyl}methyl)-1,3-thiazole-4-carboxylicacid | Rt =3.68 min[MH+]360, 362 |
| 10 | 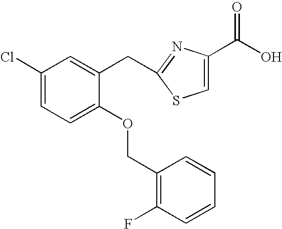 | 2-[(5-chloro-2-{[(2-fluorophenyl)methyl]oxy}phenyl)methyl]-1,3-thiazole-4-carboxylicacid | Rt =3.71 min[MH+]378, 380 |
| 11 | 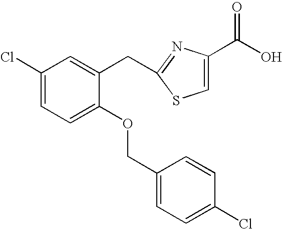 | 2-[(5-chloro-2-{[(4-chlorophenyl)methyl]oxy}phenyl)methyl]-1,3-thiazole-4-carboxylicacid | Rt =3.93 min[MH+]394 |
| 12 | 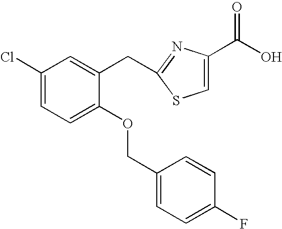 | 2-[(5-chloro-2-{[(4-fluorophenyl)methyl]oxy}phenyl)methyl]-1,3-thiazole-4-carboxylicacid | Rt =3.6 min[MH+]378 |
| 13 | 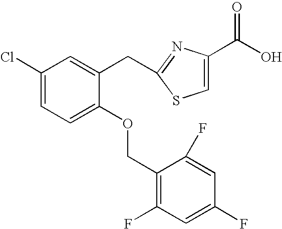 | 2-[(5-chloro-2-{[(2,4,6-trifluorophenyl)methyl]oxy}phenyl)methyl]-1,3-thiazole-4-carboxylicacid | Rt =3.64 min[MH+]414, 416 |
| 14 | 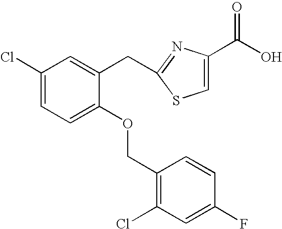 | 2-[(5-chloro-2-{[(2-chloro-4-fluorophenyl)methyl]oxy}phenyl)methyl]-1,3-thiazole-4-carboxylicacid | Rt =4.02 min[MH+]412, 416 |
| 15 | 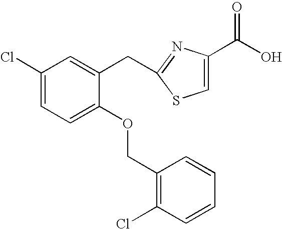 | 2-[(5-chloro-2-{[(2-chlorophenyl)methyl]oxy}phenyl)methyl]-1,3-thiazole-4-carboxylicacid | Rt =4.05 min[MH+]394, 398 |
| 16 | 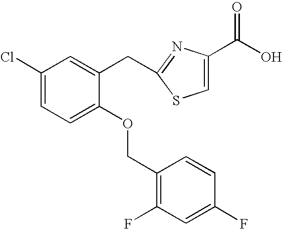 | 2-[(5-chloro-2-{[(2,4-difluorophenyl)methyl]oxy}phenyl)methyl]-1,3-thiazole-4-carboxylicacid | Rt =3.74 min[MH+]396, 398 |
| 17 | 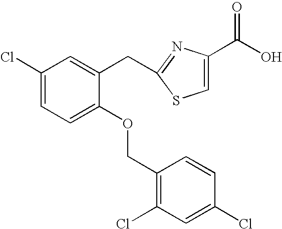 | 2-[(5-chloro-2-{[(2,4-dichlorophenyl)methyl]oxy}phenyl)methyl]-1,3-thiazole-4-carboxylicacid | Rt =4.34 min[MH+]430, 432 |

### 2-({5-Chloro-2-[(2-methylpropyl)oxy]phenyl}methyl)-1,3-oxazole-4-carboxylic acid

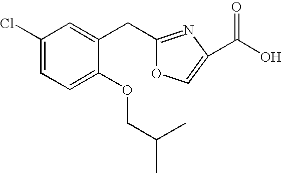

Methyl 2-({5-chloro-2-[(2-methylpropyl)oxy]phenyl}methyl)-1,3-oxazole-4-carboxylate (6.54 g, 20.2 mmol) was dissolved in methanol (35 ml) and 2M sodium hydroxide solution (10 ml) added. The solution was stirred at 50° C. for 1 h. The solvent was evaporated and the residue diluted with water, extracted with diethyl ether and the aqueous layer acidified with 2M hydrochloric acid. The aqueous layer was extracted with ethyl acetate (×3), dried and evaporated to give a white solid (5.94 g). LC/MS Rt=3.08, [MH⁺] 310.2, 312.2.

### 2-({5-Chloro-2-[(phenylmethyl)oxy]phenyl}methyl)-1,3-oxazole-4-carboxylic acid

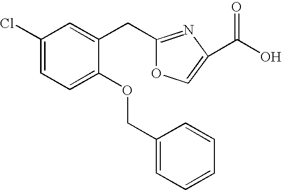

The title compound was prepared in a similar manner to 2-({5-chloro-2-[(2-methylpropyl)oxy]phenyl}methyl)-1,3-oxazole-4-carboxylic acid.

LC/MS Rt=2.89, [MH⁺] 344, 346.

### 2-[{5-Chloro-2-[(2-methylpropyl)oxy]phenyl}(difluoro)methyl]-1,3-thiazole-4-carboxylic acid

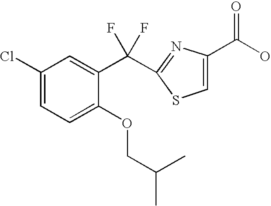

Ethyl 2-[{5-chloro-2-[(2-methylpropyl)oxy]phenyl}(difluoro)methyl]-1,3-thiazole-4-carboxylate (275 mg, 0.7 mmol) was dissolved in EtOH (6 ml) and 2M NaOH (2 ml) added, the solution was stirred at 50° C. for 2 h. Cooled, solvent evaporated, diluted with water, extracted with diethyl ether, the aqueous layer acidified with CH₃COOH. The mixture was extracted with ethyl acetate (×3), dried (MgSO₄) and evaporated to give a pale yellow solid.

LC/MS Rt=3.26 min, [MH⁺] 362.1, 364, [MH^{- ]} 360.1, 362.1

### 2-({5-Chloro-2-[(2-methylpropyl)oxy]phenyl}methyl)-1,3-thiazole-4-carboxylic acid

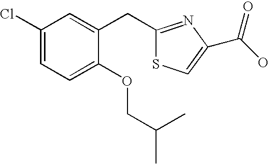

The title compound was prepared from ethyl 2-({5-chloro-2-[(2-methylpropyl)oxy]phenyl}-methyl)-1,3-thiazole-4-carboxylate using a method analogous to General Procedure 3.

LC/MS Rt=3.02 min, [MH⁺] 326.

### Ethyl 2-({5-chloro-2-[(2-methylpropyl)oxy]phenyl}amino)-1,3-thiazole-4-carboxylate

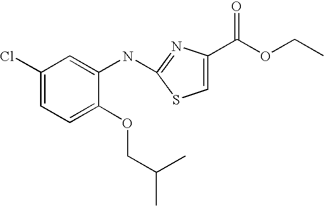

Ethyl 2-[(5-chloro-2-hydroxyphenyl)amino]-1,3-thiazole-4-carboxylate (3.0 g; 10 mmol), 1-bromo-2-methylbutane (2.74 g; 2.18 ml; 20 mmol) and potassium carbonate (5.52 g; 40 mmol) in DMF (50 ml) were stirred under argon and heated at 90° C. for 16 hours. Diethyl ether and water were added and the organic layer washed with water (×3), dried (MgSO₄) and evaporated. The residue was purified by flash chromatography, eluting with 10-30% ethyl acetate in hexane 1.69 g. LC/MS Rt=3.84 min [MH⁺] 355, 357.

### 2-({5-Chloro-2-[(2-methylpropyl)oxy]phenyl}amino)-1,3-thiazole-4-carboxylic acid

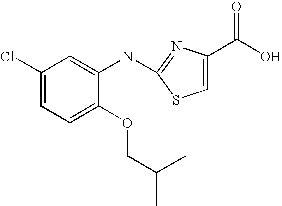

Ethyl 2-({5-chloro-2-[(2-methylpropyl)oxy]phenyl}amino)-1,3-thiazole-4-carboxylate (1.69 g, 4.76 mmol) was dissolved in ethanol (20 ml) and 2M sodium hydroxide solution (10 ml) added. The mixture was stirred at 80° C. for 30 minutes, cooled, then ethyl acetate and water added. The mixture was acidified with concentrated hydrochloric acid and the organic layer washed with water, dried (MgSO₄) and evaporated to a yellow foam 1.69 g.

LC/MS Rt=3.17 min [MH⁺] 327, 329.

### 2-({5-Chloro-2-[(2-methylpropyl)oxy]phenyl}amino)-1,3-thiazole-4-carboxamide

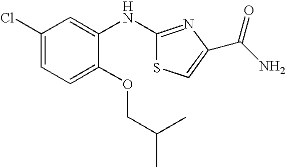

A mixture of 2-({5-chloro-2-[(2-methylpropyl)oxy]phenyl}amino)-1,3-thiazole-4-carboxylic acid (810 mg, 2.48 mmol), EDAC (570 mg, 2.98 mmol), triethylamine (606 mg, 6 mmol) and hydroxybenzotriazole ammonium salt (417 mg, 2.73 mmol) in dichloromethane (20 ml) was stirred at room temperature for 18 hours then washed with saturated sodium bicarbonate, dried (magnesium sulphate), evaporated and purified by flash chromatography on silica eluting with ethyl acetate/hexane (1:1) to give the title compound as an off-white solid (760 mg). LC/MS: Rt=3.11, [MH⁺] 326.11, 328.11

### Methyl 2-[2-({5-chloro-2-[(2-methylpropyl)oxy]phenyl}amino)-1,3-thiazol-4-yl]-1H-benzimidazole-5-carboxylate

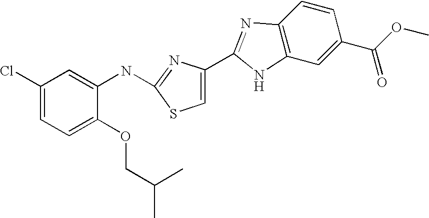

2-({5-Chloro-2-[(2-methylpropyl)oxy]phenyl}amino)-1,3-thiazole-4-carboxylic acid (1.7 g. 5.2 mmol), HOBT (773 mg, 5.8 mmol), EDAC (1.1 g, 5.8 mmol), N-methylmorpholine (1.15m, 10.4 mmol) and methyl 3,4-diaminobenzoate (950 mg, 5.8 mmol) were dissolved in dichloromethane (25 ml) and the mixture stirred at room temperature for 3 hours. The mixture was washed with 5% sodium bicarbonate solution and water, dried (MgSO₄) and evaporated. The residue was dissolved in glacial acetic acid (5 ml) and heated at 110° C. for 2 hours. The solvent was evaporated and the residue re-evaporated from toluene, then purified by flash chromatography, eluting with 10-50% ethyl acetate in hexane. The product was triturated with diethyl ether and the solid filtered and dried 980 mg.

LC/MS Rt=3.56 min [MH⁺] 457, 459.

### {2-[2-({5-Chloro-2-[(2-methylpropyl)oxy]phenyl}amino)-1,3-thiazol-4-yl]-1H-benzimidazol-5-yl}methanol

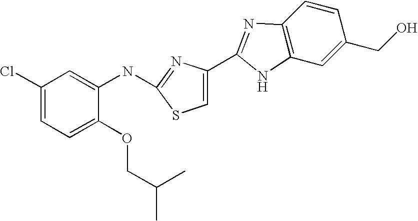

Methyl 2-[2-({5-chloro-2-[(2-methylpropyl)oxy]phenyl}amino)-1,3-thiazol-4-yl]-1H-benzimidazole-5-carboxylate (980 mg, 2.15 mmol) was dissolved in THF (10 ml) and stirred under argon. 1M Lithium aluminium hydride in diethyl ether (2.25 ml, 2.25 mmol) was added carefully at room temperature over 5 minutes. The reaction was stirred at room temperature for 1 hour then a few drops of water carefully added. After effervescence had subsided, ethyl acetate and water were added. The mixture was filtered through Hyflo to remove insoluble material and the organic layer washed with water, dried (MgSO₄) and evaporated. The residue was purified by flash chromatography, eluting with 10-50% ethyl acetate in hexane to give a yellow oil 850 mg. LC/MS Rt=2.49 min [MH⁺] 429, 431.

### 2-[2-({5-Chloro-2-[(2-methylpropyl)oxy]phenyl}amino)-1,3-thiazol-4-yl]-1H-benzimidazole-5-carbaldehyde

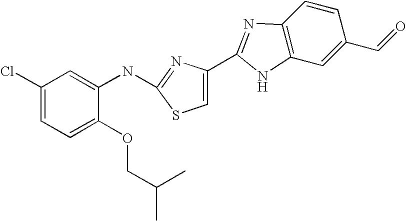

{2-[2-({5-Chloro-2-[(2-methylpropyl)oxy]phenyl}amino)-1,3-thiazol-4-yl]-1H-benzimidazol-5-yl}methanol (850 mg, 1.98 mmol) was dissolved in dichloromethane (15 ml) and Dess-Martin periodinane (899 mg, 2.17 mmol) added. The reaction was stirred at room temperature for 1 hour. The mixture was diluted with dichloromethane and washed with 5% sodium thiosulfate solution and water, dried (MgSO₄) and evaporated. The residue was purified by flash chromatography, eluting with 5-30% ethyl acetate in hexane 470 mg.

LC/MS Rt=3.51 min [MH⁺] 427, 429.

### General Procedure 4:

### Example 18

### Sodium 2-[(5-chloro-2-{[(2,4,5-trifluorophenyl)methyl]oxy}phenyl)methyl]-1,3-thiazole-4-carboxylate

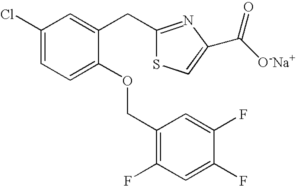

Ethyl 2-[(5-chloro-2-{[(2,3,6-trifluorophenyl)methyl]oxy}phenyl)methyl]-1,3-thiazole-4-carboxylate (86 mg, 0.195 mmol) was dissolved in ethanol (1 ml) and 2M sodium hydroxide solution (0.5 ml) was added. The mixture was heated at 120° C. for 10 minutes in a Smithcreator®microwave. After cooling, the reaction was diluted with water and extracted with ethyl acetate (×2). The combined organics were washed with water, dried (MgSO₄) and evaporated. The residue was triturated with iso-hexane and diethyl ether and the product filtered and dried in vacuo (18 mg). LC/MS Rt=3.69 min, [MH⁺] 414, 416.

The following examples were prepared by general procedure 4 from the appropriate intermediates.

| Example | | Name | Data |
|---|---|---|---|
| 19 | 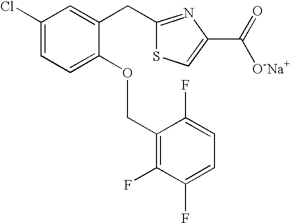 | Sodium 2-[(5-chloro-2-{[(2,3,6-trifluorophenyl)methyl]oxy}phenyl)methyl]-1,3-thiazole-4-carboxylate | LC/MSRt = 3.63 min,[MH⁺] 414,416. |
| 20 | 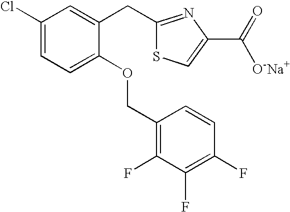 | Sodium 2-[(5-chloro-2-{[(2,3,4-trifluorophenyl)methyl]oxy}phenyl)methyl]-1,3-thiazole-4-carboxylate | LC/MSRt = 3.78 min,[MH⁺] 414,416. |
| 21 | 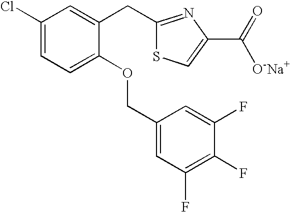 | Sodium 2-[(5-chloro-2-{[(3,4,5-trifluorophenyl)methyl]oxy}phenyl)methyl]-1,3-thiazole-4-carboxylate | LC/MSRt = 3.71 min,[MH⁺] 414,416. |
| 22 | 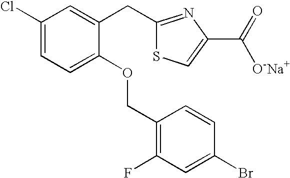 | Sodium 2-[(2-{[(4-bromo-2-fluorophenyl)methyl]oxy}-5-chlorophenyl)methyl]-1,3-thiazole-4-carboxylate | LC/MSRt = 4.04 min,[MH⁺] 458,460. |
| 23 | 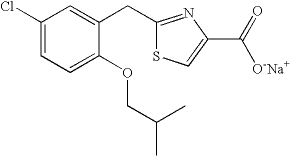 | Sodium 2-({5-chloro-2-[(2-methylpropyl)oxy}phenyl}methyl)-1,3-thiazole-4-carboxylate | LC/MSRt = 3.58 min,[MH⁺] 458,460. |

### General Procedure 5

### Example 24

### Sodium 2-{[2-[(phenylmethyl)oxy]-5-(trifluoromethyl)phenyl]methyl}-1,3-thiazole-4-carboxylate

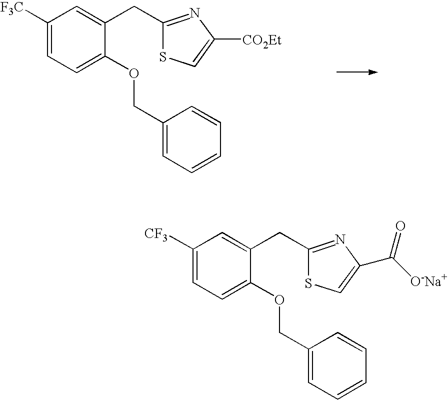

Ethyl 2-{[2-[(phenylmethyl)oxy]-5-(trifluoromethyl)phenyl]methyl}-1,3-thiazole-4-carboxylate was dissolved in ethanol and excess sodium hydroxide added (either 2M sodium hydroxide solution or solid NaOH followed by H₂O). The solution was stirred from 50° C. to 80° C. from 1 h to 3 h. In some cases solid precipitated and this was filtered off and dried. When solid did not precipitate, the solvent was evaporated and the residue partitioned between ethyl acetate and water or just diluted with ethyl acetate and water. The organic layer was dried (MgSO₄) and evaporated.

The following examples were prepared by general procedure 5 from the appropriate intermediates.

| Example | | Name | Data |
|---|---|---|---|
| 24 | 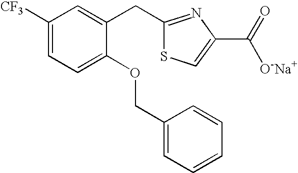 | Sodium 2-{[2-[(phenylmethyl)oxy]-5-(trifluoromethyl)phenyl]methyl}-1,3-thiazole-4-carboxylate | LC/MSRt = 3.27 min,[MH⁺] 394 |
| 25 | 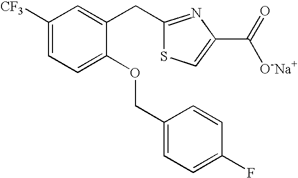 | Sodium 2-{[2-{[(4-fluorophenyl)methyl]oxy}-5-(trifluoromethyl)phenyl]methyl}-1,3-thiazole-4-carboxylate | LC/MSRt = 3.14 min,[MH⁺] 412. |
| 26 | 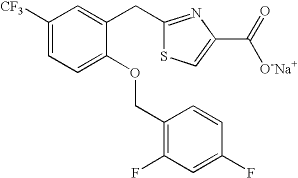 | Sodium 2-{[2-{[(2,4-difluorophenyl)methyl]oxy}-5-(trifluoromethyl)phenyl]methyl}-1,3-thiazole-4-carboxylate | LC/MSRt = 3.28 min,[MH⁺] 430. |
| 27 | 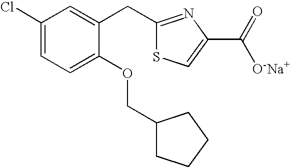 | Sodium 2-({5-chloro-2-[(cyclopentylmethyl)oxy]phenyl}methyl)-1,3-thiazole-4-carboxylate | LC/MSRt = 4.08 min,[MH⁺] 352,354. |
| 28 | 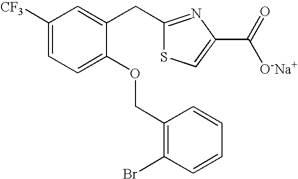 | Sodium 2-{[2-{[(2-bromophenyl)methyl]oxy}-5-(trifluoromethyl)phenyl]methyl}-1,3-thiazole-4-carboxylate | LC/MSRt = 3.95 min,[MH⁺] 472,474. |
| 29 | 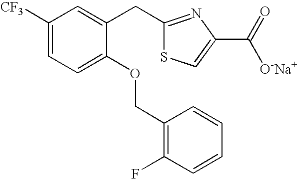 | Sodium 2-{[2-{[(2-fluorophenyl)methyl]oxy}-5-(trifluoromethyl)phenyl]methyl}-1,3-thiazole-4-carboxylate | LC/MSRt = 3.65 min,[MH⁺] 412. |
| 30 | 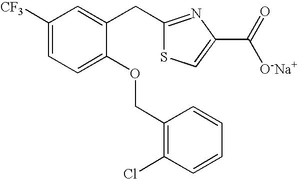 | Sodium 2-{[2-{[(2-chlorophenyl)methyl]oxy}-5-(trifluoromethyl)phenyl]methyl}-1,3-thiazole-4-carboxylate | LC/MSRt = 3.83 min,[MH⁺] 428,430. |
| 31 | 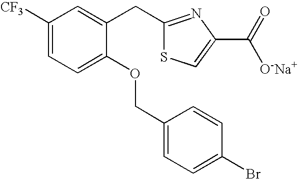 | Sodium 2-{[2-{[(4-bromophenyl)methyl]oxy}-5-(trifluoromethyl)phenyl]methyl}-1,3-thiazole-4-carboxylate | LC/MSRt = 3.91 min,[MH⁺] 472,474. |
| 32 | 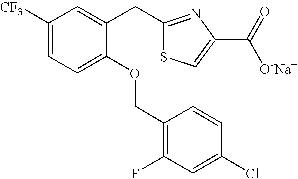 | Sodium 2-{[2-{[(4-chloro-2-fluorophenyl)methyl]oxy}-5-(trifluoromethyl)phenyl]methyl}-1,3-thiazole-4-carboxylate | LC/MSRt = 3.90 min,[MH⁺] 446,448. |
| 33 | 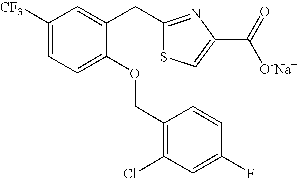 | Sodium 2-{[2-{[(2-chloro-4-fluorophenyl)methyl]oxy}-5-(trifluoromethyl)phenyl]methyl}-1,3-thiazole-4-carboxylate | LC/MSRt = 3.91 min,[MH⁺] 446,448. |
| 34 | 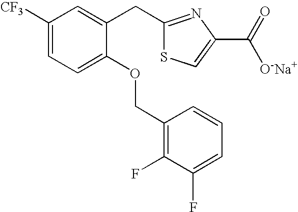 | Sodium 2-{[2-{[(2,3-difluorophenyl)methyl]oxy}-5-(trifluoromethyl)phenyl]methyl}-1,3-thiazole-4-carboxylate | LC/MSRt = 3.69 min,[MH⁺] 430. |
| 35 | 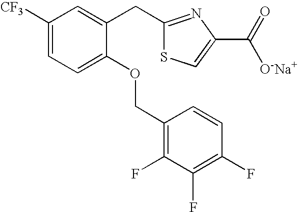 | Sodium 2-{[5-(trifluoromethyl)-2-{[(2,3,4-trifluorophenyl)methyl]oxy}phenyl)methyl]-1,3-thiazole-4-carboxylate | LC/MSRt = 3.74 min,[MH⁺] 448. |
| 36 | 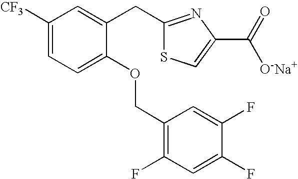 | Sodium 2-[(5-(trifluoromethyl)-2-{[(2,4,5-trifluorphenyl)methyl]oxy}phenyl)methyl]-1,3-thiazole-4-carboxylate | LC/MSRt = 3.67 min,[MH⁺] 448. |
| 37 | 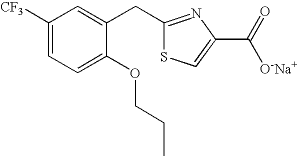 | Sodium 2-{[2-(propyloxy)-5-(trifluoromethyl)phenyl]methyl}-1,3-thiazole-4-carboxylate | LC/MSRt = 3.51 min,[MH⁺] 346. |
| 38 | 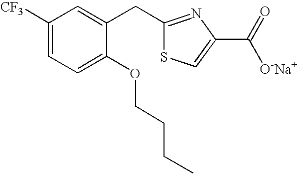 | Sodium 2-{[2-(butyloxy)-5-(trifluoromethyl)phenyl]methyl}-1,3-thiazole-4-carboxylate | LC/MSRt = 3.70 min,[MH⁺] 360. |
| 39 | 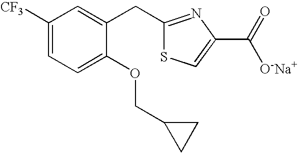 | Sodium 2-{[2-[(cyclopropylmethyl)oxy]-5-(trifluoromethyl)phenyl]methyl}-1,3-thiazole-4-carboxylate | LC/MSRt = 3.50 min,[MH⁺] 358. |
| 40 | 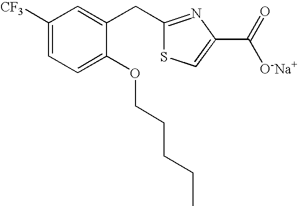 | Sodium 2-{[2-(pentyloxy)-5-(trifluoromethyl)phenyl]methyl}-1,3-thiazole-4-carboxylate | LC/MSRt = 3.89 min,[MH⁺] 374. |
| 41 | 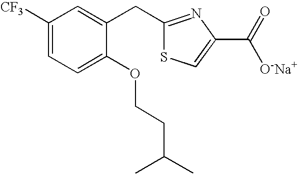 | Sodium 2-{[2-[(3-methylbutyl)oxy]-5-(trifluoromethyl)phenyl]methyl}-1,3-thiazole-4-carboxylate | LC/MSRt = 3.85 min,[MH⁺] 374. |
| 42 | 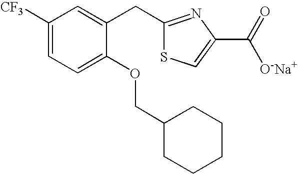 | Sodium 2-{[2-[(cyclohexylmethyl)oxy]-5-(trifluoromethyl)phenyl]methyl}-1,3-thiazole-4-carboxylate | LC/MSRt = 4.13 min,[MH⁺] 400. |
| 43 | 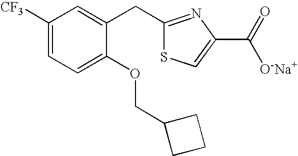 | Sodium 2-{[2-[(cyclobutylmethyl)oxy]-5-(trifluoromethyl)phenyl]methyl}-1,3-thiazole-4-carboxylate | LC/MSRt = 3.79 min,[MH⁺] 372. |
| 44 | 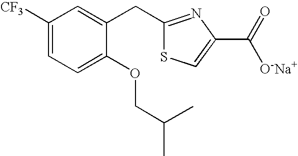 | Sodium 2-{[2-[(2-methylpropyl)oxy]-5-(trifluoromethyl)phenyl]methyl}-1,3-thiazole-4-carboxylate | LC/MSRt = 3.67 min,[MH⁺] 360. |
| 45 | 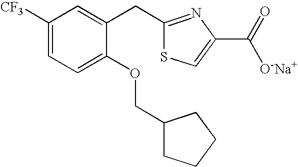 | Sodium 2-{[2-[(cyclopentylmethyl)oxy]-5-(trifluoromethyl)phenyl]methyl}-1,3-thiazole-4-carboxylate | LC/MSRt = 4.02 min,[MH⁺] 386. |
| 46 | 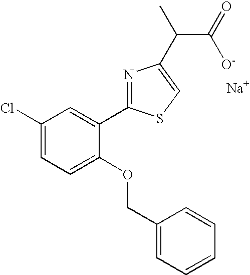 | Sodium 2-(2-{5-chloro-2-[(phenylmethyl)oxy]phenyl}-1,3-thiazol-4-yl)propanoate | LC/MSRt = 3.88 min,[MH⁺] 374 |
| 47 | 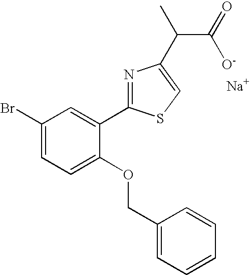 | Sodium 2-(2-{5-bromo-2-[(phenylmethyl)oxy]phenyl}-1,3-thiazol-4-yl)propanoate | LC/MSRt = 3.82 min,[MH⁺] 418,420. |
| 48 | 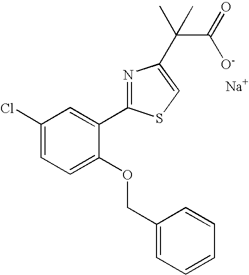 | Sodium 2-(2-{5-chloro-2-[(phenylmethyl)oxy]phenyl}-1,3-thiazol-4-yl)-2-methylpropanoate | LC/MSRt = 4.04 min,[MH⁺] 388. |
| 49 | 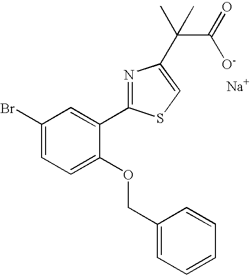 | Sodium 2-(2-{5-bromo-2-[(phenylmethyl)oxy]phenyl}-1,3-thiazol-4-yl)-2-methylpropanoate | LC/MSRt = 4.10 min,[MH⁺]432, 434. |
| 50 | 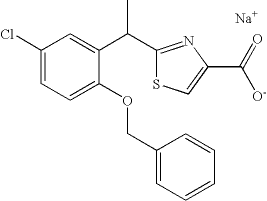 | Sodium 2-(1-{5-chloro-2-[(phenylmethyl)oxy]phenyl}-1,3-thiazole-4-carboxylate | LC/MSRt = 3.77 min,[MH⁺] 374. |
| 51 | 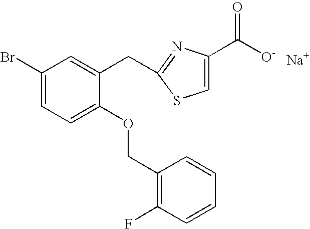 | Sodium 2-[(5-bromo-2-{[(2-fluorophenyl)methyl]oxy}phenyl)methyl]-1,3-thiazole-4-carboxylate | Rt = 3.4 min[MH+]424, 425 |
| 52 | 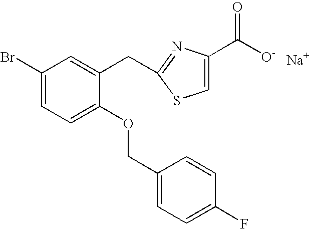 | Sodium 2-[(5-bromo-2-{[(4-fluorophenyl)methyl]oxy}phenyl)methyl]-1,3-thiazole-4-carboxylate | Rt = 3.52 min[MH+]424 |
| 53 | 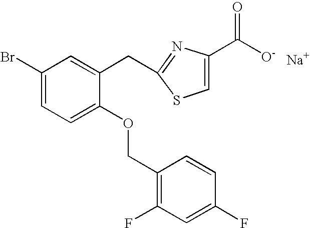 | Sodium 2-[(5-bromo-2-{[(2,4-difluorophenyl)methyl]oxy}phenyl)methyl]-1,3-thiazole-4-carboxylate | Rt = 3.58 min[MH+]442, 443 |
| 54 | 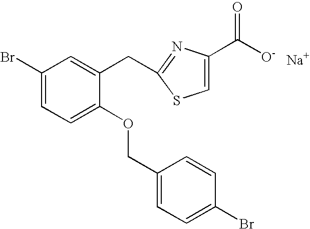 | Sodium 2-[(5-bromo-2-{[(4-bromophenyl)methyl]oxy}phenyl)methyl]-1,3-thiazole-4-carboxylate | Rt = 3.71 min[MH+]484, 486 |
| 55 | 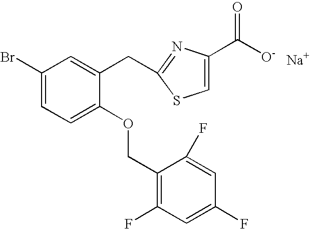 | Sodium 2-[(5-bromo-2-{[(2,4,6-trifluorophenyl)methyl]oxy}phenyl)methyl]-1,3-thiazole-4-carboxylate | Rt = 3.46 min[MH+]460, 461 |
| 56 | 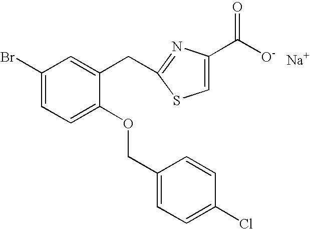 | Sodium 2-[(5-bromo-2-{[(4-chlorophenyl)methyl]oxy}phenyl)methyl]-1,3-thiazole-4-carboxylate | Rt = 3.7 min[MH+]440, 442 |
| 57 | 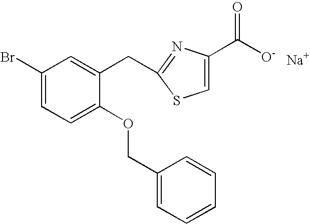 | Sodium 2-({5-bromo-2-[(phenylmethyl)oxy]phenyl}methyl)-1,3-thiazole-4-carboxylate | Rt = 3.77 min[MH+]406, 407 |
| 58 | 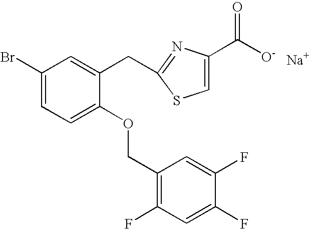 | Sodium 2-[(5-bromo-2-{[(2,4,5-trifluorophenyl)methyl]oxy}phenyl)methyl]-1,3-thiazole-4-carboxylate | Rt = 3.49 min[MH+]460, 461 |
| 59 | 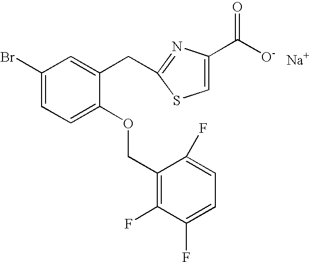 | Sodium 2-[(5-bromo-2-{[(2,3,6-trifluorophenyl)methyl]oxy}phenyl)methyl]-1,3-thiazole-4-carboxylate | Rt = 3.3 min[MH+]460, 461 |
| 60 | 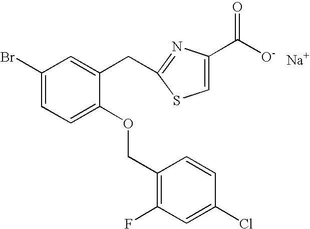 | Sodium 2-[(5-bromo-2-{[(4-chloro-2-fluorophenyl)methyl]oxy}phenyl)methyl]-1,3-thiazole-4-carboxylate | Rt = 3.83 min[MH+]458, 460 |
| 61 | 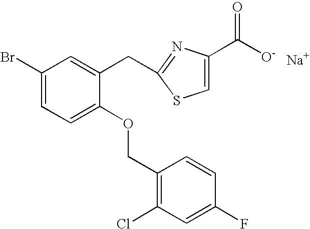 | Sodium 2-[(5-bromo-2-{[(2-chloro-4-fluorophenyl)methyl]oxy}phenyl)methyl]-1,3-thiazole-4-carboxylate | Rt = 3.9 min[MH+]458, 460 |
| 62 | 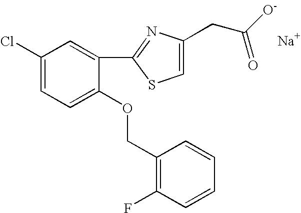 | Sodium [2-(5-chloro-2-{[(2-fluorophenyl)methyl]oxy}phenyl)-1,3-thiazol-4-yl]acetate | Rt = 3.51 min[MH+]378, 380 |
| 63 | 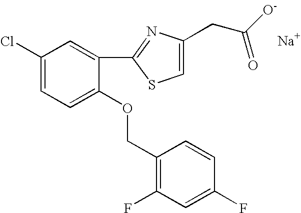 | Sodium [2-(5-chloro-2-{[(2,4-difluorophenyl)methyl]oxy}phenyl)-1,3-thiazol-4-yl]acetate | Rt = 3.58 min[MH+]396, 398 |
| 64 | 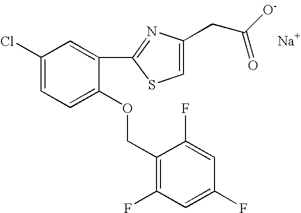 | Sodium [2-(5-chloro-2-{[(2,4,6-trifluorophenyl)methyl]oxy}phenyl)-1,3-thiazol-4-yl]acetate | Rt = 3.5 min[MH+]414, 416 |
| 65 | 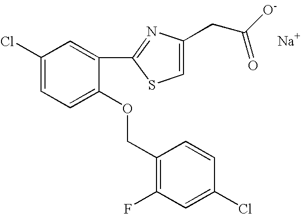 | Sodium [2-(5-chloro-2-{[(4-chloro-2-fluorophenyl)methyl]oxy}phenyl)-1,3-thiazol-4-yl]acetate | Rt = 3.75 min[MH+]412, 415 |
| 66 | 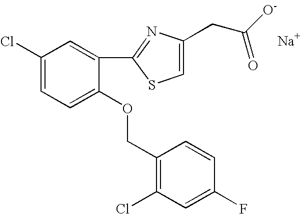 | Sodium [2-(5-chloro-2-{[(2-chloro-4-fluorophenyl)methyl]oxy}phenyl)-1,3-thiazol-4-yl]acetate | Rt = 3.75 min[MH+]412, 416 |
| 67 | 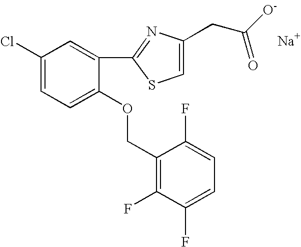 | Sodium [2-(5-chloro-2-{[(2,3,6-trifluorophenyl)methyl]oxy}phenyl)-1,3-thiazol-4-yl]acetate | Rt = 3.5 min[MH+]414, 416 |
| 68 | 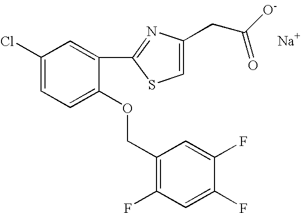 | Sodium [2-(5-chloro-2-{[(2,4,5-trifluorophenyl)methyl]oxy}phenyl)-1,3-thiazol-4-yl]acetate | Rt = 3.59 min[MH+]414, 416 |
| 69 | 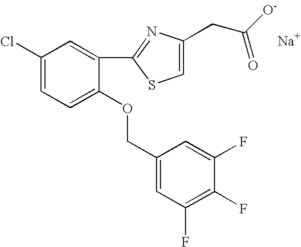 | Sodium [2-(5-chloro-2-{[(3,4,5-trifluorophenyl)methyl]oxy}phenyl)-1,3-thiazol-4-yl]acetate | Rt = 3.62 min[MH+]414, 416 |
| 70 | 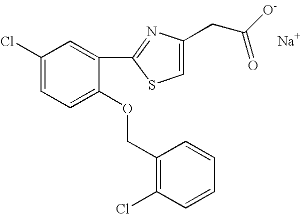 | Sodium [2-(5-chloro-2-{[(2-chlorophenyl)methyl]oxy}phenyl)-1,3-thiazol-4-yl]acetate | Rt = 3.73 min[MH+]394, 398 |
| 71 | 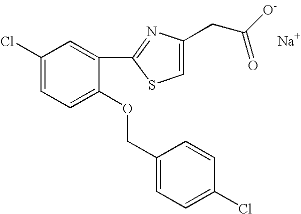 | Sodium [2-(5-chloro-2-{[(4-chlorophenyl)methyl]oxy}phenyl)-1,3-thiazol-4-yl]acetate | Rt = 3.5 min[MH+]394 |
| 72 | 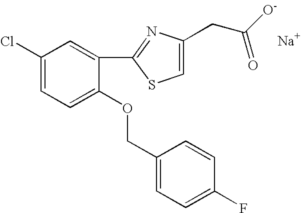 | Sodium [2-(5-chloro-2-{[(4-fluorophenyl)methyl]oxy}phenyl)-1,3-thiazol-4-yl]acetate | Rt = 3.38 min[MH+]378 |
| 73 | 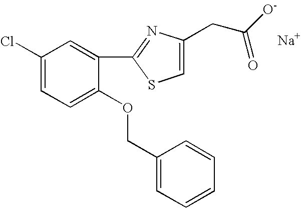 | Sodium [2-{5-chloro-2-[(phenylmethyl)oxy]phenyl}-1,3-thiazol-4-yl)acetate | Rt = 3.65 min[MH+]360, 362 |
| 74 | 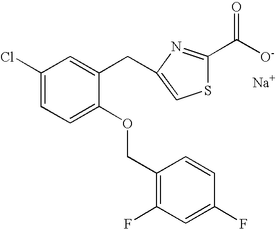 | Sodium 4-[(5-chloro-2-{[(2,4-difluorophenyl)methyl]oxy}phenyl)methyl]-1,3-thiazole-2-carboxylate | Rt = 4.29 min[MH+]396, 398 |
| 75 | 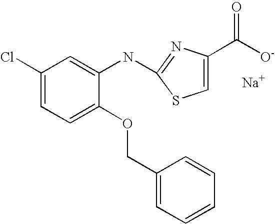 | Sodium 2-({5-chloro-2-[(phenylmethyl)oxy]phenyl}amino)-1,3-thiazole-4-carboxylate | Rt = 3.77 min[MH+]361, 363 |
| 76 | 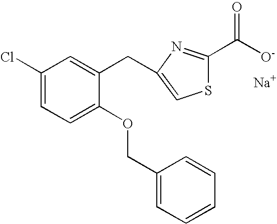 | Sodium 4-({5-chloro-2-[(phenylmethyl)oxy]phenyl}methyl)-1,3-thiazole-2-carboxylate | Rt = 3.85 min,[MH+]360, 362. |
| 77 | 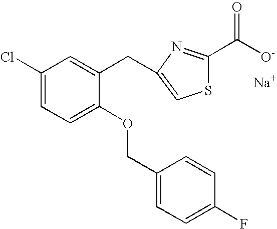 | Sodium 4-[(5-chloro-2-{[(4-fluorophenyl)methyl]oxy}phenyl)methyl]-1,3-thiazole-2-carboxylate | Rt = 4.1 min,[MH+]378. |
| 78 | 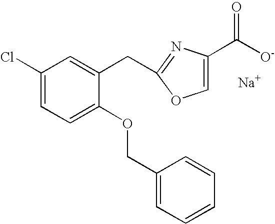 | Sodium 2-({5-chloro-2-[(phenylmethyl)oxy]phenyl}methyl)-1,3-oxazole-4-carboxylate | Rt = 3.40 min[MH+]344. |
| 79 | 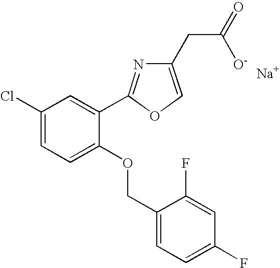 | Sodium [2-(5-chloro-2-{[(2,4-difluorophenyl)methyl]oxy}phenyl)-1,3-oxazol-4-yl]acetate | Rt = 3.51 min[MH⁺]380. |
| 80 | 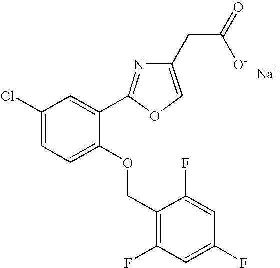 | Sodium [2-(5-chloro-2-{[(2,4,6-trifluorophenyl)methyl]oxy}phenyl)-1,3-oxazol-4-yl]acetate | Rt = 3.35 min[MH⁺]398. |
| 81 | 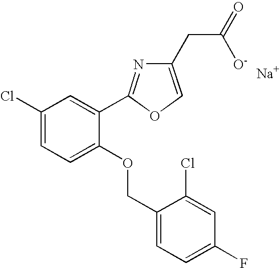 | Sodium [2-(5-chloro-2-{[(2-chloro-4-fluorophenyl)methyl]oxy}phenyl)-1,3-oxazol-4-yl]acetate | Rt = 3.83 min[MH⁺]396. |
| 82 | 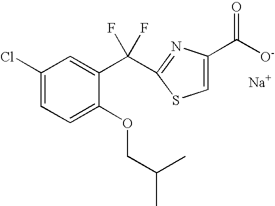 | Sodium 2-[{5-chloro-2-[(2-methylpropyl)oxy]phenyl}(difluoro)methyl]-1,3-thiazole-4-carboxylate | LC/MSRt = 3.26 min,[MH⁺] 362,364, [MH]360.1, 362.1 |

### Example 83

### Sodium 4-[(5-chloro-2-{[(2-chlorophenyl)methyl]oxy}phenyl)methyl]-1,3-thiazole-2-carboxylate

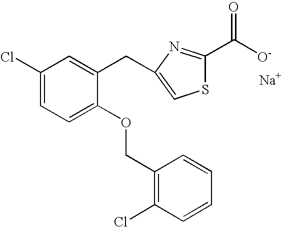

4-[(5-chloro-2-{[(2-chlorophenyl)methyl]oxy}phenyl)methyl]-1,3-thiazole-2-carboxamide (54 mg, 0.137 mmol) was dissolved in ethanol (3 ml) and water (1 ml), sodium hydroxide (22 mg, 0.55 mmol) was added and the solution heated at 60° C. for 8 hours, then at 90° C. for another 10 hours. The mixture was cooled, evaporated, diluted with water and extracted with ethyl acetate (×3). The organic phase was dried, filtered and concentrated to yield the title compound. LC/MS Rt=4.76, [MH⁺] 394.

The following compounds were prepared in a similar manner to sodium 4-[(5-chloro-2-{[(2-chlorophenyl)methyl]oxy}phenyl)methyl]-1,3-thiazole-2-carboxylate from the appropriate intermediates

| Example | | Name | Data |
|---|---|---|---|
| 84 | 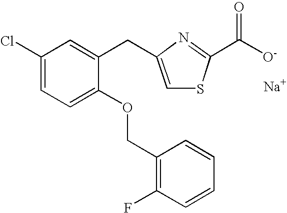 | Sodium 4-[(5-chloro-2-{[(2-fluorophenyl)methyl]oxy}-phenyl)methyl]-1,3-thiazole-2-carboxylate | Rt = 4.12min [MH+]378 |
| 85 | 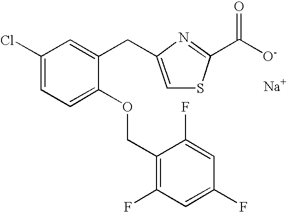 | Sodium 4-[(5-chloro-2-{[(2,4,6-trifluorophenyl)methyl]-oxy}phenyl)methyl]-1,3-thiazole-2-carboxylate | Rt = 4.0 min[MH+]414, 416 |
| 86 | 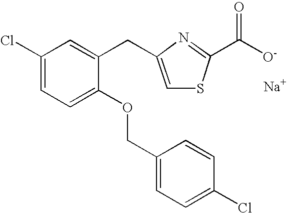 | Sodium 4-[(5-chloro-2-{[(4-chlorophenyl)methyl]-oxy}phenyl)methyl]-1,3-thiazole-2-carboxylate | Rt = 4.3 min[MH+] 394 |
| 87 | 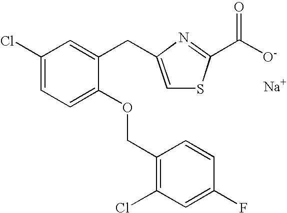 | Sodium 4-[(5-chloro-2-{[(2-chloro-4-fluorophenyl)methyl]oxy}-phenyl)methyl]-1,3-thiazole-2-carboxylate | Rt = 4.52min [MH+]412 |
| 88 | 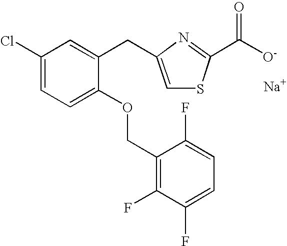 | Sodium 4-[(5-chloro-2-{[(2,3,6-trifluorophenyl)methyl]-oxy}phenyl)methyl]-1,3-thiazole-2-carboxylate | Rt = 4.02min [MH+]414, 416 |
| 89 | 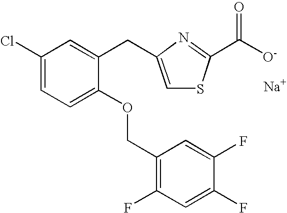 | Sodium 4-[(5-chloro-2-{[(2,4,5-trifluorophenyl)methyl]-oxy}phenyl)methyl]-1,3-thiazole-2-carboxylate | Rt = 4.07min [MH+]414, 416 |

### 2-({-5-Chloro-2-[(phenylmethyl)oxy]phenyl}methyl)-1,3-thiazole-4-carbonitrile

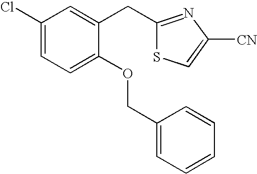

2-({5-Chloro-2-[(phenylmethyl)oxy]phenyl}methyl)-1,3-thiazole-4-carboxamide (632 mg, 1.76 mmol) was dissolved in phosphorus(III)oxychloride (6 ml) and the solution heated at 90° C. for 1 h.

The solution was evaporated and the residue dissolved in diethyl ether and washed with water and 5% sodium bicarbonate solution, dried (MgSO₄) and evaporated. The orange oil was triturated with diethyl ether and iso-hexane and the cream solid filtered and dried in vacuo (600 mg). LC/MS Rt=3.78 min, [MH⁻] 341, 343.

### 2-({-5-Chloro-2-[(2-methylpropyl)oxy]phenyl}methyl)-1,3-thiazole-4-carbonitrile

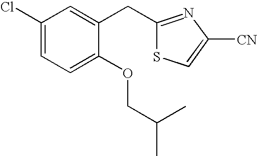

The title compound was prepared in a similar manner to 2-({5-chloro-2-[(phenylmethyl)oxy]phenyl}methyl)-1,3-thiazole-4-carbonitrile using 2-({5-chloro-2-[(2-methylpropyl)oxy]phenyl}methyl)-1,3-thiazole-4-carboxamide.

LC/MS Rt=3.57 min, [MH⁺] 307.

### 2-({5-Chloro-2-[(2-methylpropyl)oxy]phenyl}methyl)-1,3-oxazole-4-carbonitrile

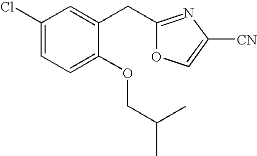

A solution of 2-({5-chloro-2-[(2-methylpropyl)oxy]phenyl}methyl)-1,3-oxazole-4-carboxamide (1.16 g, 3.76 mmol) in phosphorus oxychloride (5 ml) was stirred and heated at 75° C. for one hour then poured onto ice, extracted with ether and the organic phase washed with water and saturated sodium bicarbonate solution then dried with magnesium sulphate and evaporated to dryness. The residue was purified by chromatography on a Biotage eluting with (1:9) ethyl acetate/hexane to give the title compound as a colourless gum (990 mg). LC/MS Rt=3.56, [MH⁺] 291.

### 2-({5-Chloro-2-[(2-methylpropyl)oxy]phenyl}amino)-1,3-thiazole-4-carbonitrile

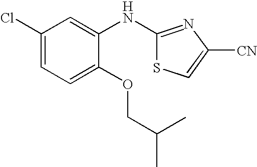

A mixture of 2-({5-chloro-2-[(2-methylpropyl)oxy]phenyl}amino)-1,3-thiazole-4-carboxamide (690 mg, 2.12 mmol) and phosphorus oxychloride (5 ml) was stirred and heated to reflux for one minute then cooled to room temperature. The solution was poured onto ice, extracted with ether and the organic phase washed with 2M sodium hydroxide, dried (MgSO₄), evaporated and purified by flash chromatography on silica eluting with ethyl acetate/hexane (15:85) to give the title compound as a white solid (510 mg).

LC/MS: Rt=3.74, [MH⁺] 308.17, 310.17

### Methyl 2-({5-chloro-2-[(2-methylpropyl)oxy]phenyl}amino)-1,3-thiazole-4-carboximidoate hydrochloride

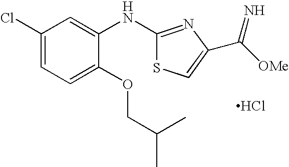

60% Sodium hydride (10 mg, 0.25 mmol) was added to a solution of 2-({5-chloro-2-[(2-methylpropyl)oxy]phenyl}amino)-1,3-thiazole-4-carbonitrile (500 mg, 1.62 mmol) in methanol (20 ml) and left at room temperature for 20 hours then evaporated, dissolved in ether/water and the organic phase dried (MgSO₄), and filtered. The resulting solution was treated with 1M hydrogen chloride in ether producing a gummy yellow precipitate. The solvent was decanted and the residue dissolved in dichloromethane/ether and evaporated to give the title compound as a yellow foam (580 mg). LC/MS: Rt=2.49, [MH⁺] 340.19, 342.22.

### Ethyl 2-({5-chloro-2-[(phenylmethyl)oxy]phenyl}methyl)-1,3-thiazole-4-carboximidoate hydrochloride

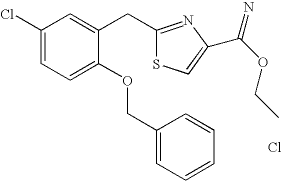

A solution of 2-({5-chloro-2-[(phenylmethyl)oxy]phenyl}methyl)-1,3-thiazole-4-carbonitrile (250 mg, 0.73 mmol) and ethanol (40 mg, 0.9 mmol) in dry diethyl ether (10 ml) was saturated with hydrogen chloride gas and allowed to stand for four days at 4° C. The solvent was evaporated and the residue triturated with dry diethyl ether to give the title compound as a colourless solid 200 mg, 67%.

### Ethyl 2-({5-chloro-2-[(2-methylpropyl)oxy]phenyl}methyl)-1,3-thiazole-4-carboximidoate hydrochloride

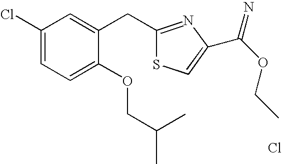

The title compound was prepared in a similar manner to ethyl 2-({5-chloro-2-[(phenylmethyl)oxy]phenyl}methyl)-1,3-thiazole-4-carboximidoate hydrochloride using 2-({5-chloro-2-[(2-methylpropyl)oxy]phenyl}methyl)-1,3-thiazole-4-carbonitrile.

### Methyl 2-({5-chloro-2-[(2-methylpropyl)oxy]phenyl}methyl)-1,3-oxazole-4-carboximidoate hydrochloride

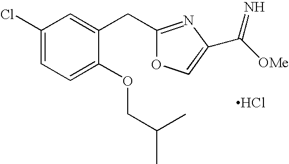

60% Sodium hydride (20 mg, 0.5 mmol) was added to a solution of 2-({5-chloro-2-[(2-methylpropyl)oxy]phenyl}methyl)-1,3-oxazole-4-carbonitrile (990 mg, 3.4 mmol) in methanol (15 ml) and left at room temperature for 16 hours then evaporated to dryness. The residue was dissolved in ether, washed with water, dried (MgSO₄), evaporated and treated with 1M hydrogen chloride in ether (6 ml). The gum which separated crystallised on scratching and was filtered off to give the title compound as a white solid (1.05 g).

LC/MS Rt=2.89, [MH⁺] 323, 325

### Methyl 2-({5-chloro-2-[(phenylmethyl)oxy]phenyl}methyl)-1,3-thiazole-4-carboximidoate hydrochloride

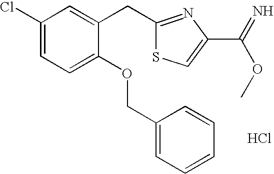

Sodium hydride, 60% in oil (100 mg, 2.5 mmol) was added portionwise to a solution of 2-({5-chloro-2-[(phenylmethyl)oxy]phenyl}methyl)-1,3-thiazole-4-carbonitrile (1.30 g, 3.82 mmol) in methanol (25 ml). The mixture was stirred at room temperature overnight. The solvent was evaporated, and the residue dissolved in diethyl ether (25 ml). The solution was washed with water (2×10 ml). The organic phase was dried, then treated with 1.0M hydrogen chloride in diethyl ether (10 ml) to give the title compound as colourless solid 900 mg. LC/MS Rt=2.69 min, [MH⁺] 373.

### Methyl 2-({5-chloro-2-[(phenylmethyl)oxy]phenyl}methyl)-1,3-thiazole-4-carboximidoate

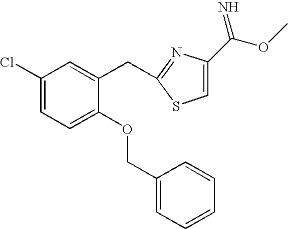

2-({5-Chloro-2-[(phenylmethyl)oxy]phenyl}methyl)-1,3-thiazole-4-carbonitrile (1.32 g, 3.87 mmol) was dissolved in methanol (20 ml) and NaOMe (90 mg) was added, the mixture was stirred at room temperature overnight. The solvent was evaporated, redissolved with methanol and more NaOMe added, stirred for another 4 hours. The solvent was evaporated and the residue triturated with diethyl ether/hexane mixture to give the title compound as a brown solid. LC/MS Rt=2.68 min, [MH⁺] 373.1, 375.2

### Example 90

### 2-({5-Chloro-2-[(phenylmethyl)oxy]phenyl}methyl)-1,3-thiazole-4-carboxamide

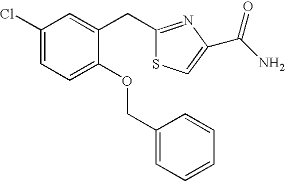

2-({5-Chloro-2-[(phenylmethyl)oxy]phenyl}methyl)-1,3-thiazole-4-carboxylic acid (108 mg, 0.3 mmol) was dissolved in dichloromethane (5 ml) and ammonium 1H-1,2,3-benzotriazol-1-olate (50 mg, 0.33 mmol), N-(3-dimethylaminopropyl)-N′-ethylcarbodiimide (60 mg, 0.33 mmol) and N-methylmorpholine (66 μl, 0.33 mmol) were added. The mixture was stirred at room temperature for 2 h. The solvent was evaporated and the residue dissolved in ethyl acetate which was washed with 5% sodium bicarbonate solution (×2) and brine, dried (MgSO₄) and evaporated. The residue was purified by flash chromatography, eluting with 20-50% ethyl acetate in iso-hexane to leave a white solid (57 mg).

LC/MS Rt=3.17 min, [MH⁺] 359, 361.

### 2-({5-Chloro-2-[(2-methylpropyl)oxy]phenyl}methyl)-1,3-thiazole-4-carboxamide

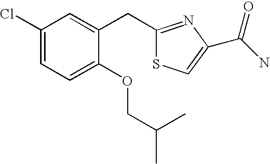

The title compound was prepared in a similar manner to 2-({5-chloro-2-[(phenylmethyl)oxy]phenyl}methyl)-1,3-thiazole-4-carboxamide using 2-({5-chloro-2-[(2-methylpropyl)oxy]phenyl}methyl)-1,3-thiazole-4-carboxylic acid.

LC/MS Rt=2.99 min, [MH⁺] 325.

### 2-({5-Chloro-2-[(2-methylpropyl)oxy]phenyl}methyl)-1,3-oxazole-4-carboxamide

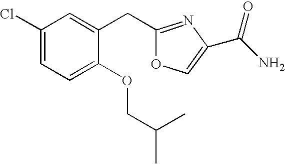

Oxalyl chloride (1 ml) was added to a solution of 2-({5-chloro-2-[(2-methylpropyl)oxy]phenyl}methyl)-1,3-oxazole-4-carboxylic acid (1.21 g, 3.91 mmol) and DMF (1 drop) in dichloromethane (15 ml) and left at room temperature for one hour. The resulting solution was evaporated to dryness and azeotroped with toluene then dissolved in dichloromethane (20 ml) and aqueous ammonia (6 ml) added with vigorous stirring. The organic layer was separated, washed with brine, dried (MgSO₄), and evaporated to dryness to give the title compound as a white solid (1.16 g). LC/MS Rt=3.00, [MH⁺] 309.

### Example 91

### 2-({5-Chloro-2-[(phenylmethyl)oxy]phenyl}methyl)-N-phenyl-1,3-thiazole-4-carboxamide

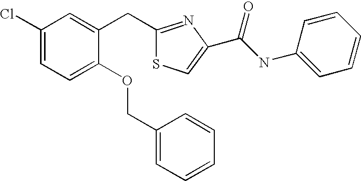

2-({5-Chloro-2-[(phenylmethyl)oxy]phenyl}methyl)-1,3-thiazole-4-carboxylic acid (72 mg, 0.2 mmol) was dissolved in tetrahdrofuran (2 ml). Aniline (20 μl, 0.22 mmol), N-(3-dimethylaminopropyl)-N′-ethylcarbodiimide (44 mg, 0.24 mmol) and N,N-dimethyl-4-pyridinamine (2 mg) were added. The mixture was stirred at room temperature for 2 h. The solvent was evaporated and the residue dissolved in ethyl acetate. The solution was washed with 5% sodium bicarbonate solution, 1M hydrochloric acid and brine, dried (MgSO₄) and evaporated. The residue was purified by flash chromatography, eluting with 2% methanol in dichloromethane. The product was triturated with iso-hexane and diethyl ether to leave a cream solid (16 mg). LC/MS Rt=4.11 min, [MH⁻] 435, 437.

The following examples were prepared in a similar manner to 2-({5-chloro-2-[(phenylmethyl)oxy]phenyl}methyl)-N-phenyl-1,3-thiazole-4-carboxamide from the appropriate intermediates.

| Example | | Name | Data |
|---|---|---|---|
| 92 | 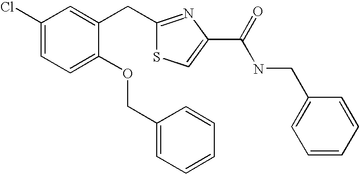 | 2-({5-Chloro-2-[(phenylmethyl)oxy]phenyl}methyl)-N-(phenylmethyl)-1,3-thiazole-4-carboxamide | LC/MS Rt =3.95 min,[MH⁺] 449,451. |
| 93 | 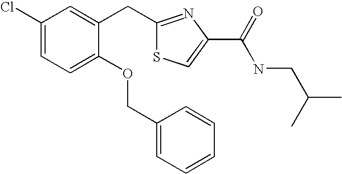 | 2-({5-Chloro-2-[(phenylmethyl)oxy]phenyl}methyl)-N-(2-methylpropyl)-1,3-thiazole-4-carboxamide | LC/MS Rt =3.89 min,[MH⁺] 415,417. |
| 94 | 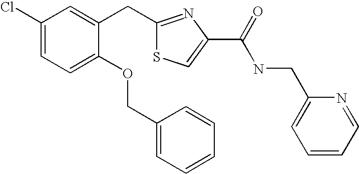 | 2-({5-Chloro-2-[(phenylmethyl)oxy]phenyl}methyl)-N-(2-pyridinylmethyl)-1,3-thiazole-4-carboxamide | LC/MS Rt =3.59 min,[MH⁺] 450,452. |
| 95 | 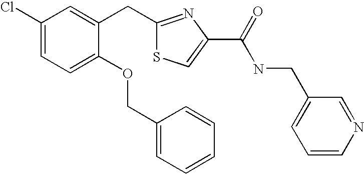 | 2-({5-Chloro-2-[(phenylmethyl)oxy]phenyl}methyl)-N-(3-pyridinylmethyl)-1,3-thiazole-4-carboxamide | LC/MS Rt =3.43 min,[MH⁺] 450,452. |
| 96 | 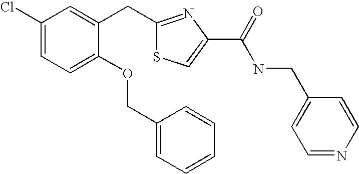 | 2-({5-Chloro-2-[(phenylmethyl)oxy]phenyl}methyl)-N-(4-pyridinylmethyl)-1,3-thiazole-4-carboxamide | LC/MS Rt =3.39 min,[MH⁺] 450,452. |
| 97 | 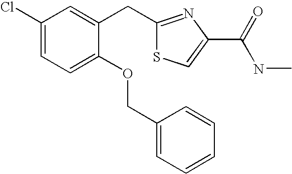 | 2-({5-Chloro-2-[(phenylmethyl)oxy]phenyl}methyl)-N-methyl-1,3-thiazole-4-carboxamide | LC/MS Rt =3.47 min,[MH⁺] 373,375. |
| 98 | 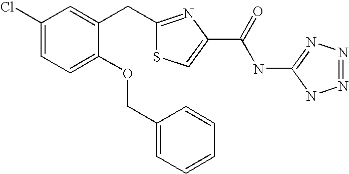 | 2-({5-Chloro-2-[(phenylmethyl)oxy]phenyl}methyl)-N-1H-tetrazol-5-yl-1,3-thiazole-4-carboxamide | LC/MS Rt =3.66 min,[MH⁺] 427,429. |
| 99 | 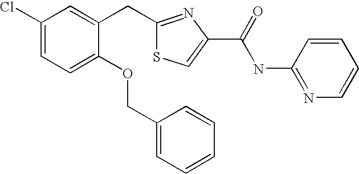 | 2-({5-Chloro-2-[(phenylmethyl)oxy]phenyl}methyl)-N-2-pyridinyl-1,3-thiazole-4-carboxamide | LC/MS Rt =4.08 min,[MH⁺] 436,438. |

### Example 100

### 2-({5-Chloro-2-[(phenylmethyl)oxy]phenyl}methyl)-N-(phenylsulfonyl)-1,3-thiazole-4-carboxamide

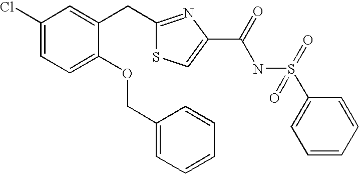

2-({5-Chloro-2-[(phenylmethyl)oxy]phenyl}methyl)-1,3-thiazole-4-carboxylic acid (130 mg, 0.361 mmol) was dissolved in tetrahydrofuran (1 ml) and dichloromethane (1 ml). Benzenesulphonamide (85 mg, 0.542 mmol), N-(3-dimethylaminopropyl)-N′-ethylcarbodiimide (79 mg, 0.434 mmol) and N,N-dimethyl-4-pyridinamine (5 mg) were added. The mixture was stirred at room temperature for 3 h. The mixture was diluted with dichloromethane and washed with 5% sodium bicarbonate solution (×2), 1M hydrochloric acid and brine, dried (MgSO₄) and evaporated. The residue was purified by flash chromatography, eluting with 2% methanol in dichloromethane. The product was triturated with iso-hexane and diethyl ether and the cream solid dried in vacuo (102 mg).

LC/MS Rt=3.92 min, [MH⁺] 499, 501.

The following examples were prepared in a similar manner to 2-({5-chloro-2-[(phenylmethyl)oxy]phenyl}methyl)-N-(phenylsulfonyl)-1,3-thiazole-4-carboxamide from the appropriate intermediates.

| Example | | Name | Data |
|---|---|---|---|
| 101 | 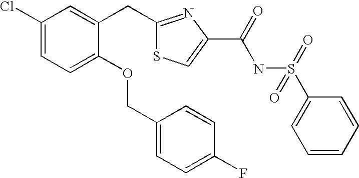 | 2-[(5-Chloro-2-{[(4-fluorophenyl)methyl]-oxy}phenyl)methyl]-N-(phenylsulfonyl)-1,3-thiazole-4-carboxamide | LC/MS Rt =3.89 min,[MH⁺] 517,519, |
| 102 | 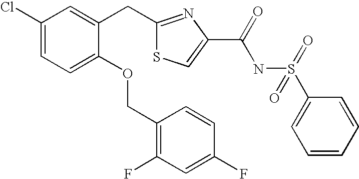 | 2-[(5-Chloro-2-{[(2,4-difluorophenyl)methyl]oxy}phenyl)methyl]-N-(phenylsulfonyl)-1,3-thiazole-4-carboxamide | LC/MS Rt =3.95 min,[MH⁺] 535,537. |
| 103 | 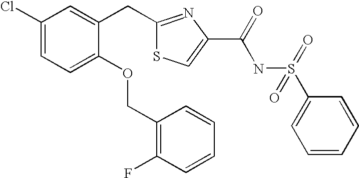 | 2-[(5-Chloro-2-{[(2-fluorophenyl)methyl]-oxy}phenyl)methyl]-N-(phenylsulfonyl)-1,3-thiazole-4-carboxamide | LC/MS Rt =3.92 min,[MH⁺] 517,519. |
| 104 | 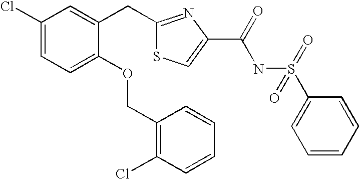 | 2-[(5-Chloro-2-{[(2-chlorophenyl)methyl]-oxy}phenyl)methyl]-N-(phenylsulfonyl)-1,3-thiazole-4-carboxamide | LC/MS Rt =4.11 min,[MH⁺] 533,535. |
| 105 | 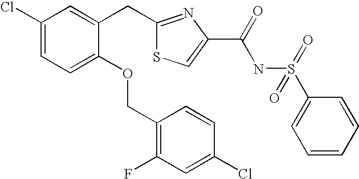 | 2-[(5-Chloro-2-{[(4-chloro-2-fluorophenyl)methyl]-oxy}phenyl)methyl]-N-(phenylsulfonyl)-1,3-thiazole-4-carboxamide | LC/MS Rt =4.14 min,[MH⁺] 551,553. |
| 106 | 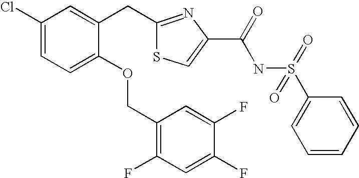 | 2-[(5-Chloro-2-{[(2,4,5-trifluorophenyl)methyl]oxy}phenyl)methyl]-N-(phenylsulfonyl)-1,3-thiazole-4-carboxamide | LC/MS Rt =3.93 min,[MH⁺] 553,555. |
| 107 | 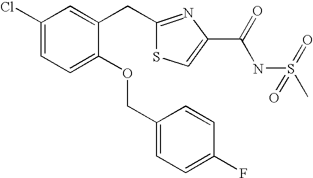 | 2-[(5-Chloro-2-{[(4-fluorophenyl)methyl]-oxy)phenyl)methyl]-N-(methylsulfonyl)-1,3-thiazole-4-carboxamide | LC/MS Rt =3.42 min,[MH⁻] 453,455. |
| 108 | 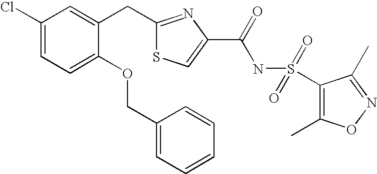 | 2-({5-Chloro-2-[(phenylmethyl)oxy]-phenyl}methyl)-N-[(3,5-dimethyl-4-isoxazolyl)sulfonyl]-1,3-thiazole-4-carboxamide | LC/MS Rt =3.71 min,[MH⁻] 518,520. |

### Example 109

### 5-[2-({5-Chloro-2-[(phenylmethyl)oxy]phenyl}methyl)-1,3-thiazol-4-yl]-1H-tetrazole

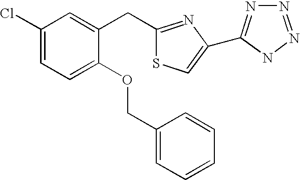

2-({5-Chloro-2-[(phenylmethyl)oxy]phenyl}methyl)-1,3-thiazole-4-carbonitrile (170 mg, 0.5 mmol) was dissolved in dimethylformamide (3 ml) and sodium azide (98 mg, 1.5 mmol) and ammonium chloride (80 mg, 1.5 mmol) added. The mixture was heated at 100° C. for 64 h. The cooled mixture was diluted with ethyl acetate and washed with water (×4). The organic layer was dried (MgSO₄) and evaporated. The residue was triturated with diethyl ether and the cream powder filtered and dried in vacuo (127 mg).

LC/MS Rt=3.84 min, [MH⁻] 384, 386.

### Example 110

### 1,1-Dimethylethyl [2-({5-chloro-2-[(phenylmethyl)oxy]phenyl}methyl)-1,3-thiazol-4-yl]carbamate

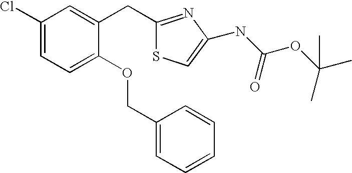

2-({5-Chloro-2-[(phenylmethyl)oxy]phenyl}methyl)-1,3-thiazole-4-carboxylic acid (10.79 g, 30 mmol) was stirred in 2-methyl-2-propanol (100 ml) under nitrogen. Triethylamine (4.62 g, 33 mmol) and diphenylphosphoryl azide (7.12 ml, 33 mmol) were added and the mixture was heated to reflux for 6 h. The solvent was evaporated and the residue purified by flash chromatography, eluting with 15% ethyl acetate in iso-hexane to give the title compound as a cream solid (8.94 g). LC/MS Rt=4.18 min, [MH] 431, 433. A second crop was obtained from slightly impure chromatography fractions which were evaporated and triturated with diethyl ether filtered and dried (1.1 g)

### Example 111

### 1,1-Dimethylethyl [2-({5-chloro-2-[(2-methylpropyl)oxy]phenyl}methyl)-1,3-thiazol-4-yl]carbamate

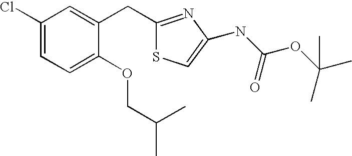

The title compound was prepared in a manner similar to 1,1-dimethylethyl [2-({5-chloro-2-[(phenylmethyl)oxy]phenyl}methyl)-1,3-thiazol-4-yl]carbamate. LC/MS Rt=4.04 min, [MH] 397.

### 2-({5-Chloro-2-[(phenylmethyl)oxy]phenyl}methyl)-1,3-thiazol-4-amine

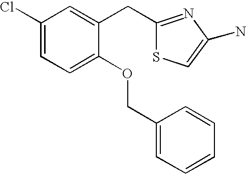

1,1-Dimethylethyl [2-({5-chloro-2-[(phenylmethyl)oxy]phenyl}methyl)-1,3-thiazol-4-yl]carbamate (8.94 g, 20.75 mmol) was dissolved in acetonitrile (50 ml) and 4-toluenesulphonic acid (11.83 g, 62.26 mmol) added. The mixture was stirred and heated to reflux for 30 minutes, cooled and evaporated. The residue was dissolved in ethyl acetate and washed with 5% sodium bicarbonate solution (×2) and water, dried (MgSO₄) and evaporated. The brown residue was purified by flash chromatography, eluting with 1-2% methanol in dichloromethane to give the title compound as an orange oil which crystallised on standing (3.48 g). LC/MS Rt=3.40 min, [MH⁻] 331, 333.

### Methyl 4-({[2-({5-chloro-2-[(phenylmethyl)oxy]phenyl}methyl)-1,3-thiazol-4-yl]amino}carbonyl)benzoate

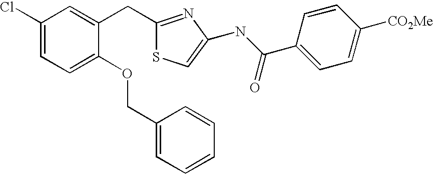

A solution of 2-({5-chloro-2-[(phenylmethyl)oxy]phenyl}methyl)-1,3-thiazol-4-amine (600 mg, 2 mmol), methyl 4-(chlorocarbonyl)benzoate (436 mg, 2.2 mmol) and triethylamine (255 mg, 2.5 mmol) in dichloromethane (10 ml) was stirred at room temperature for 1 hour. The solvent was evaporated and the residue purified by flash chromatography eluting with 25% ethyl acetate in hexane to give the title compound as an off white solid.

LC/MS Rt=3.76 min, [MH⁺] 1493.

### Example 112

### N-[2-({5-chloro-2-[(phenylmethyl)oxy]phenyl}methyl)-1,3-thiazol-4-yl]-4-(hydroxymethyl)benzamide

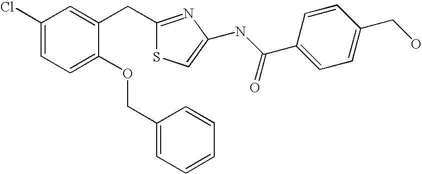

A solution of methyl 4-({[2-({5-chloro-2-[(phenylmethyl)oxy]phenyl}methyl)-1,3-thiazol-4-yl]amino}carbonyl)benzoate (299 mg, 0.4 mmol) in dry tetrahydrofuran (2 ml) was cooled to 0° C. under argon and treated with 1.0M lithium aluminium hydride in diethyl ether (0.40 ml, 0.4 mmol). The reaction mixture was stirred at 0° C. for 30 mins., then quenched with water (10 ml). The mixture was extracted with ethyl acetate (2×10 ml). The combined extracts were dried and evaporated. The residue was triturated with ethyl acetate/hexane to give the title compound as a colourless solid 160 mg, 86%. LC/MS Rt=3.33 min, [MH⁺] 465.

### 2-({-5-Chloro-2-[(phenylmethyl)oxy]phenyl}methyl)-N-[4-(hydroxymethyl)phenyl]-1,3-thiazole-4-carboxamide

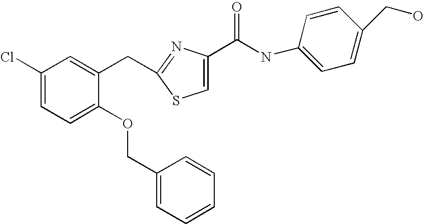

2-({5-Chloro-2-[(phenylmethyl)oxy]phenyl}methyl)-1,3-thiazole-4-carboxylic acid (600 mg, 1.67 mmol) was dissolved in DCM, 4-methylmorpholine (0.36 ml, 3.3 mmol), EDAC (383 mg, 2 mmol), HOBt (270 mg, 2 mmol) and (4-aminophenyl)methanol (406 mg, 3.3 mmol) were added and the solution was stirred under argon overnight. The solvent was evaporated; the residue was dissolved in ethyl acetate and washed with saturated NaHCO₃ solution, 0.5N HCl, brine and water. The organic phase was dried (MgSO₄) and evaporated to give a brown oil that was purified on a Biotage using 1-3% methanol in dichloromethane; the residue was triturated with diethyl ether and purified on a Flash Master II using 20-60% ethyl acetate in hexane. Trituration with Et₂O gave the title compound as a white solid.

LC/MS Rt=3.36 min, [MH⁺] 465, 467 [MH⁻] 463.1, 465.2

### N-[2-({5-Chloro-2-[(phenylmethyl)oxy]phenyl}methyl)-1,3-thiazol-4-yl]-4-formylbenzamide

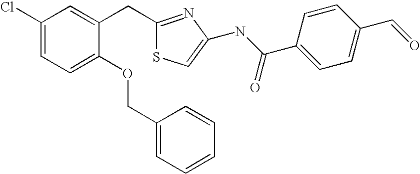

Dess-Martin periodinane (146 mg, 0.34 mmol) was added to a stirred solution of N-[2-({5-chloro-2-[(phenylmethyl)oxy]phenyl}methyl)-1,3-thiazol-4-yl]-4-(hydroxymethyl)benzamide (160 mg, 0.34 mmol) in dry dichloromethane (2 ml) and stirred at room temperature for 1 hour. 10% aqueous sodium thiosulphate (10 ml) and saturated sodium hydrogen carbonate (10 ml) were added. The mixture was extracted with dichloromethane (2×10 ml). The combined extracts were dried and evaporated to give the title compound as a yellow oil.

LC/MS Rt=3.61 min, [MH⁺] 463.

### 2-({-5-Chloro-2-[(phenylmethyl)oxy]phenyl}methyl)-N-(4-formylphenyl)-1,3-thiazole-4-carboxamide

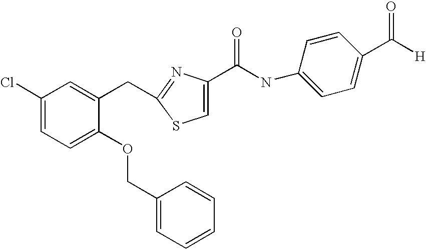

Dess-Martin periodinane (433 mg, 1.02 mmol) was added to a stirred solution 2-({5-chloro-2-[(phenylmethyl)oxy]phenyl}methyl)-N-[4-(hydroxymethyl)phenyl]-1,3-thiazole-4-carboxamide (473 mg, 1.02 mmol) in dichloromethane (20 ml) and a drop of water and stirred at room temperature for 1 hour. The reaction was quenched with 10% aqueous sodium thiosulphate, washed with saturated sodium hydrogen carbonate, and the organic phase dried (MgSO₄) and evaporated to give the title compound as solid (520 mg).

LC/MS Rt=3.7 min, [MH⁺] 463.1, 465 [MH⁻] 461.1, 463.1

### Example 113

### N-[2-({5-Chloro-2-[(phenylmethyl)oxy]phenyl}methyl)-1,3-thiazol-4-yl]-4-(1-piperidinylmethyl)benzamide

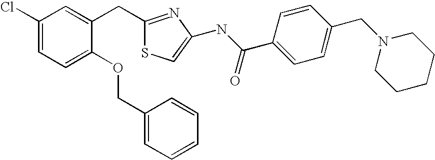

A mixture of N-[2-({5-chloro-2-[(phenylmethyl)oxy]phenyl}methyl)-1,3-thiazol-4-yl]-4-formylbenzamide (157 mg, 0.34 mmol), piperidine (29 mg, 0.34 mmol), acetic acid (20 mg, 0.34 mmol) and sodium triacetoxyborohydride (71 mg, 0.34 mmol) in dry dichloromethane (2 ml) was stirred at room temperature overnight. The solvent was evaporated and the residue purified by flash chromatography eluting with 3% methanol in dichloromethane to give the title compound as a yellow oil.

LC/MS Rt=2.53 min, [MH⁺] 532.

The following examples were prepared in a similar manner to N-[2-({5-Chloro-2-[(phenylmethyl)oxy]phenyl}methyl)-1,3-thiazol-4-yl]-4-(1-piperidinylmethyl)benzamide from the appropriate intermediates

| Example | | Name | LC/MS |
|---|---|---|---|
| 114 | 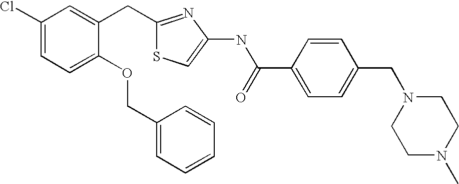 | N-[2-({5-chloro-2-[(phenylmethyl)oxy]-phenyl}methyl)-1,3-thiazol-4-yl]-4-[(4-methyl-1-piperazinyl)methyl]-benzamide | Rt =2.46 min.,[MH⁺] 546. |
| 115 | 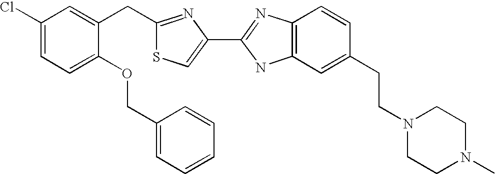 | 2-[2-({5-chloro-2-[(phenylmethyl)oxy]-phenyl}methyl)-1,3-thiazol-4-yl]-5-[2-(4-methyl-1-piperazinyl)ethyl]-1H-benzimidazole | Rt = 2.17min.,[MH⁺] 558 |
| 116 | 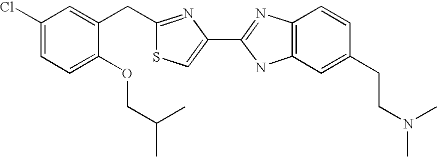 | (2-{2-[2-({5-Chloro-2-[(2-methylpropyl)oxy]-phenyl}methyl)-1,3-thiazol-4-yl]-1H-benzimidazol-5-yl}ethyl)dimethylamine | Rt = 2.33min.,[MH⁺] 469 |
| 117 | 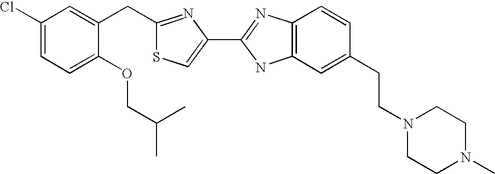 | 2-[2-({5-Chloro-2-[(2-methylpropyl)oxy]phenyl}-methyl)-1,3-thiazol-4-yl]-5-(2-(4-methyl-1-piperazinyl)ethyl]-1H-benzimidazole | Rt = 2.27min.,[MH⁺] 524 |

### Example 118

### 2-({5-Chloro-2-[(phenylmethyl)oxy]phenyl}methyl)-N-{4-[(dimethylamino)methyl]phenyl}-1,3-thiazole-4-carboxamide hydrochloride

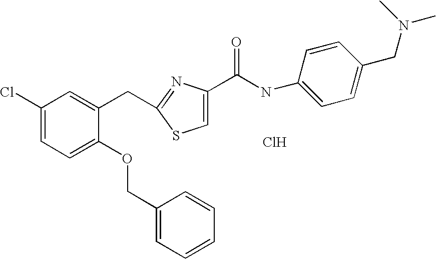

Sodium triacetoxyborohydride (69 mg, 0.32 mmol) was added to a stirred solution of 2-({5-chloro-2-[(phenylmethyl)oxy]phenyl}methyl)-N-(4-formylphenyl)-1,3-thiazole-4-carboxamide (75 mg, 0.16 mmol) and dimethylamine (18 μl, 0.32 mmol) in THF (4 ml). The mixture was stirred at room temperature over the weekend. More sodium triacetoxyborohydride was added and the mixture stirred for another 20 h. Diluted with H₂O and extracted with ethyl acetate (×3), combined organics dried and evaporated. The residue was first purified on a Flash Master 11 (8% of methanol in dichloromethane), followed by purification on a MDAP. The oil obtained was treated with 1M HCl in Et₂O to give a white solid.

LC/MS Rt=2.44 min, [MH⁻] 490.2, 492.1

The following compounds were prepared in a similar manner to 2-({5-chloro-2-[(phenylmethyl)oxy]phenyl}methyl)-N-{4-[(dimethylamino)methyl]phenyl}-1,3-thiazole-4-carboxamide hydrochloride from the appropriate intermediates

| Example | | Name | LC/MS |
|---|---|---|---|
| 119 | 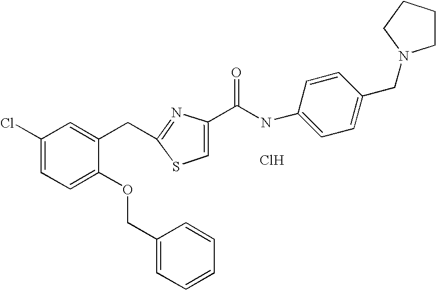 | 2-({5-Chloro-2-[(phenylmethyl)oxy]phenyl}-methyl)-N-[4-(1-pyrrolidinylmethyl)phenyl]-1,3-thiazole-4-carboxamidehydrochloride | LC/MS Rt =2.55 min., [MH⁺]518.3 [MH⁻]516.1, 518.1 |
| 120 | 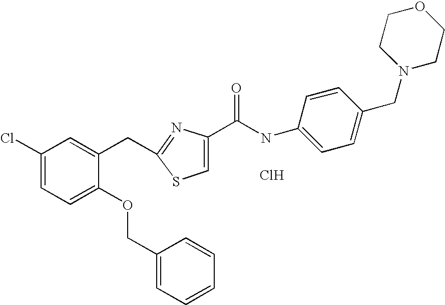 | 2-({5-Chloro-2-[(phenylmethyl)oxy]phenyl}-methyl)-N-[4-(4-morpholinylmethyl)phenyl]-1,3-thiazole-4-carboxamidehydrochloride | LC/MS Rt = 2.5min, [MH⁺]534.3 [MH⁻]532.2, 534.2 |
| 121 | 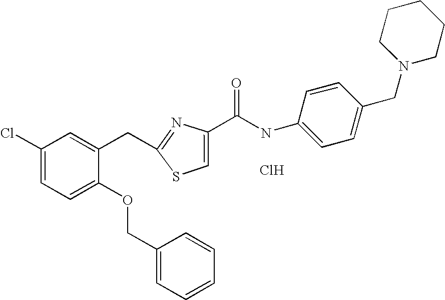 | 2-({5-Chloro-2-[(phenylmethyl)oxy]phenyl}-methyl)-N-[4-(1-piperidinylmethyl)phenyl]-1,3-thiazole-4-carboxamidehydrochloride | LC/MS Rt =2.56 min, [MH⁺]532.3 [MH⁻]530.2 |
| 122 | 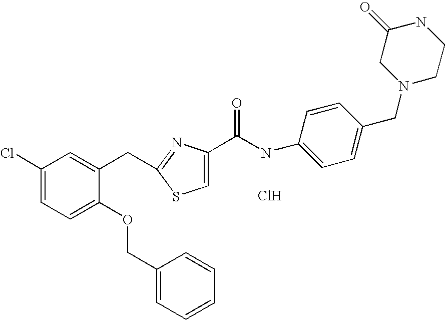 | 2-({5-Chloro-2-[(phenylmethyl)oxy]phenyl}-methyl)-N-{4-[(3-oxo-1-piperazinyl)methyl]phenyl}-1,3-thiazole-4-carboxamidehydrochloride | LC/MS Rt =2.55 min, [MH⁺]547.1 [MH⁻]545.2 |
| 123 | 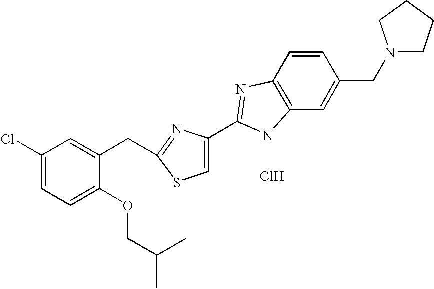 | 2-[2-({5-Chloro-2-[(2-methylpropyl)oxy]phenyl}-methyl)-1,3-thiazol-4-yl]-5-(1-pyrrolidinylmethyl)-1H-benzimidazole hydrochloride | LC/MS Rt =2.34 min, [MH⁺]481.1 [MH⁻]479.1, 481.2 |
| 124 | 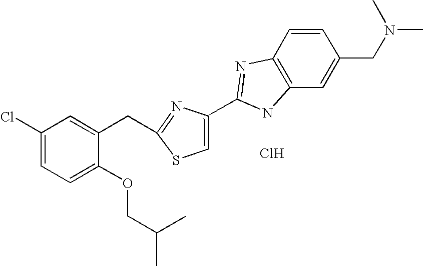 | ({2-[2-({5-Chloro-2-[(2-methylpropyl)oxy]phenyl}-methyl)-1,3-thiazol-4-yl]-1H-benzimidazol-5-yl}methyl)dimethylaminehydrochloride | LC/MS Rt =2.34 min, [MH⁻]453.1, 455.2 |
| 125 | 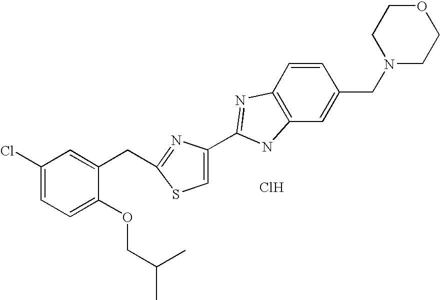 | 2-[2-({5-Chloro-2-[(2-methylpropyl)oxy]phenyl}-methyl)-1,3-thiazol-4-yl]-5-(4-morpholinylmethyl)-1H-benzimidazolehydrochloride | LC/MS Rt =2.23 min, [MH⁺]497.1 [MH⁻]495.2, 497.2 |
| 126 | 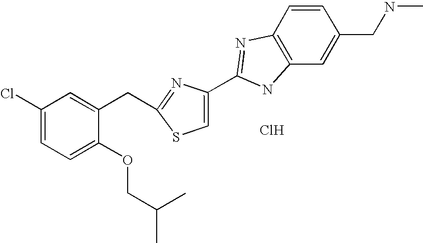 | ({2-[2-({5-Chloro-2-[(2-methylpropyl)oxy]phenyl}-methyl)-1,3-thiazol-4-yl]-1H-benzimidazol-5-yl}methyl)methylaminehydrochloride | LC/MS Rt =2.25 min, [MH⁺]441.2 [MH⁻]439.2, 441.2 |

### Example 127

### N-[2-({5-Chloro-2-[(phenylmethyl)oxy]phenyl}methyl)-1,3-thiazol-4-yl]acetamide

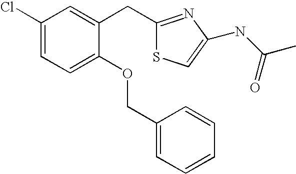

2-({5-Chloro-2-[(phenylmethyl)oxy]phenyl}methyl)-1,3-thiazol-4-amine (100 mg, 0.3 mmol) was dissolved in dichloromethane (2 ml) and triethylamine (50 μl, 0.36 mmol) added, followed by acetyl chloride (24 μl, 0.33 mmol). The reaction was stirred at room temperature for 2 h. The mixture was diluted with diethyl ether and washed with 1M hydrochloric acid, 5% sodium bicarbonate solution and water, dried (MgSO₄) and evaporated. The residue was purified by flash chromatography, eluting with 10-20% ethyl acetate in iso-hexane. The product was triturated with iso-hexane and diethyl ether to give the title compound (26 mg). LC/MS Rt=3.52 min, [MH⁻] 373, 375.

The following examples were prepared in a similar manner to N-[2-({5-chloro-2-[(phenylmethyl)oxy]phenyl}methyl)-1,3-thiazol-4-yl]acetamide from the appropriate intermediates.

| Example | | Name | Data |
|---|---|---|---|
| 128 | 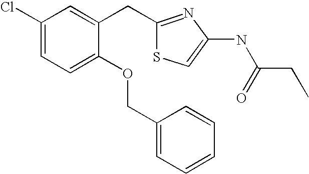 | 2-({5-Chloro-2-[(phenylmethyl)oxy]phenyl}methyl)-1,3-thiazol-4-yl]propanamide | LC/MS Rt =3.68 min,[MH⁻] 387,389. |
| 129 | 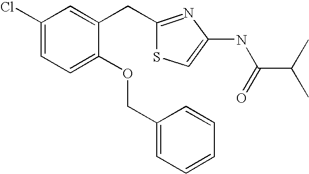 | N-[2-({5-Chloro-2-[(phenylmethyl)oxy]phenyl}methyl)-1,3-thiazol-4-yl]-2-methylpropanamide | LC/MS Rt =3.84 min,[MH⁻] 401,403. |
| 130 | 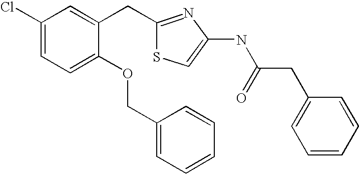 | N-[2-({5-Chloro-2-[(phenylmethyl)oxy]phenyl}methyl)-1,3-thiazol-4-yl]-2-phenylacetamide | LC/MS Rt =3.98 min,[MH⁻]449,451. |

### Example 131

### 2-[2-({5-Chloro-2-[(phenylmethyl)oxy]phenyl}methyl)-1,3-thiazol-4-yl]-1H-benzimidazole

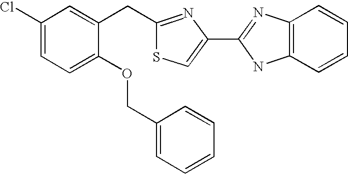

A mixture of 2-({5-chloro-2-[(phenylmethyl)oxy]phenyl}methyl)-1,3-thiazole-4-carboxylic acid (200 mg, 0.55 mmol) and 1,2-phenylenediamine (60 mg, 0.55 mmol) in phosphorus oxychloride (1 ml) was heated at 100° C. for 30 mins. The reaction mixture was cooled, and poured onto ice (50 ml). The mixture was neutralized with saturated sodium hydrogen carbonate and extracted with ethyl acetate (2×20 ml). The combined extracts were dried and evaporated, the residue was purified by chromatography, eluting with 2% methanol in dichloromethane to give the title compound as a brown solid 50 mg 20%.

LC/MS Rt=2.81 min, [MH⁺] 432.

The following compounds were made in a manner similar to 2-[2-({5-chloro-2-[(phenylmethyl)oxy]phenyl}methyl)-1,3-thiazol-4-yl]-1H-benzimidazole using the appropriate intermediates.

Hydrochloride salts were prepared by stirring a solution of benzimidazole product in 1.0M hydrogen chloride in diethyl ether (2 ml) for 15mins. The solvent was evaporated and the products were obtained by trituration of the solid with diethyl ether/hexane.

| Example | | Name | Data |
|---|---|---|---|
| 132 | 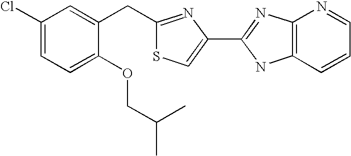 | 2-[2-({5-Chloro-2-[(2-methyl-propyl)oxy]-phenyl}methyl)-1,3-thiazol-4-yl]-1H-imidazo[4,5-b]pyridine | LC/MS Rt =3.11 min,[MH⁺] 399. |
| 133 | 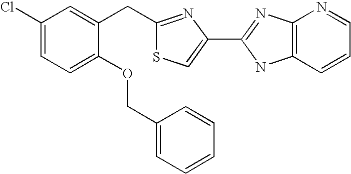 | 2-[2-({5-Chloro-2-[(phenylmethyl)oxy]phenyl}methyl)-1,3-thiazol-4-yl]-1H-imidazo[4,5-b]pyridine | LC/MS Rt =2.95 min,[MH⁺] 433. |
| 134 | 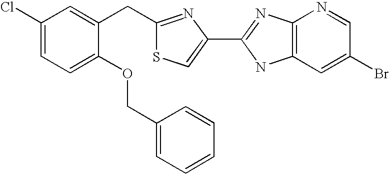 | 6-Bromo-2-[2-({5-chloro-2-[(phenylmethyl)oxy]phenyl}methyl)-1,3-thiazol-4-yl]-1H-imidazo[4,5-b]pyridine | LC/MS Rt =3.48 min,[MH⁺] 510,512. |
| 135 | 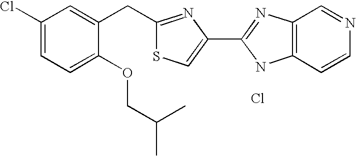 | 2-[2-({5-Chloro-2-[(2-methyl-propyl)oxy]-phenyl}methyl)-1,3-thiazol-4-yl]-1H-imidazo[4,5-c]pyridinehydrochloride | LC/MS Rt =2.29 min,[MH⁺] 399. |
| 136 | 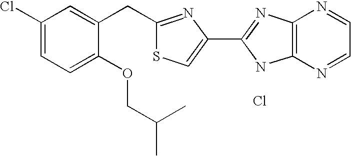 | 2-[2-({5-Chloro-2-[(2-methyl-propyl)oxy]-phenyl}methyl)-1,3-thiazol-4-yl]-1H-imidazo[4,5-b]pyrazinehydrochloride | LC/MS Rt =3.17 min,[MH⁺] 400. |
| 137 | 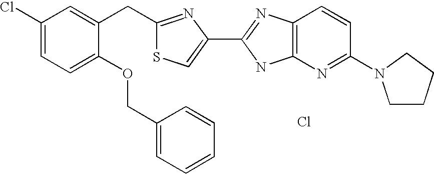 | 2-[2-({5-Chloro-2-[(phenylmethyl)oxy]phenyl}methyl)-1,3-thiazol-4-yl]-5-(1-pyrrolidinyl)-1H-imidazo[4,5-b]pyridinehydrochloride | LC/MS Rt =2.72 min,[MH⁺] 502. |
| 138 | 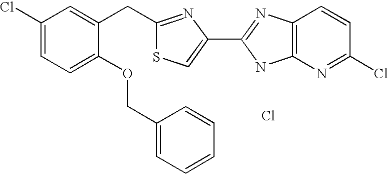 | 5-Chloro-2-[2-({5-chloro-2-[(phenylmethyl)oxy]phenyl}methyl)-1,3-thiazol-4-yl]-1H-imidazo[4,5-b]pyridinehydrochloride | LC/MS Rt =3.47 min,[MH⁺] 467. |
| 139 | 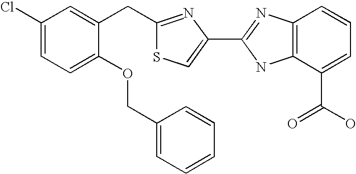 | 2-[2-({5-Chloro-2-[(phenylmethyl)oxy]phenyl}methyl)-1,3-thiazol-4-yl]-1H-benzimidazole-4-carboxylic acid | LC/MS Rt =3.24 min,[MH⁺] 476. |
| 140 | 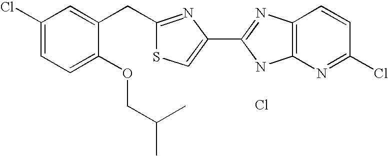 | 5-Chloro-2-[2-({5-chloro-2-[(2-methylpropyl)oxy]phenyl}methyl)-1,3-thiazol-4-yl]-1H-imidazo[4,5-b]pyridinehydrochloride | LC/MS Rt =3.52 min,[MH⁺] 432. |

### Example 141

### 2-{2-[{5-chloro-2-[(2-methylpropyl)oxy]phenyl}(difluoro)methyl]-1,3-thiazol-4-yl}-1H-benzimidazole hydrochloride

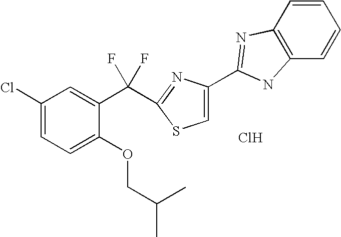

2-[{5-Chloro-2-[(2-methylpropyl)oxy]phenyl}(difluoro)methyl]-1,3-thiazole-4-carboxylic acid (125 mg, 0.345 mmol) and 1,2-phenylenediamine (37 mg, 0.345 mmol) in phosphorus oxychloride (15 ml) was heated at 100° C. for 4 h. The reaction mixture was cooled, and poured onto ice neutralized with potassium carbonate and extracted with ethyl acetate (×3). The combined extracts were dried and evaporated; the residue was purified using a FLEX. The residue was redissolved in methanol and 3 ml of 1M HCl in Et₂O added, stirred for 15 minutes, evaporated and triturated with Et₂O to give the title compound as a white solid. LC/MS Rt=3.44 min, [MH⁺] 434.2, 436.2, [MH⁻] 432.06, 434.05.

### Methyl 2-[2-({5-chloro-2-[(2-methylpropyl)oxy]phenyl}methyl)-1,3-thiazol-4-yl]-1H-benzimidazole-5-carboxylate

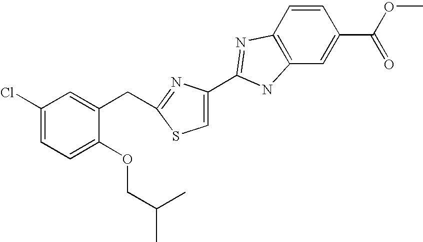

The title compound was prepared in a similar manner to 2-{2-[{5-chloro-2-[(2-methylpropyl)oxy]phenyl}(difluoro)methyl]-1,3-thiazol-4-yl}-1H-benzimidazole hydrochloride using 1.2 equivalent of the methyl 3,4-diaminobenzoate.

LC/MS Rt=3.58 min, [MH⁺] 456.1, 458, [MH⁻] 454.1, 456.1

### {2-[2-({5-Chloro-2-[(2-methylpropyl)oxy]phenyl}methyl)-1,3-thiazol-4-yl]-1H-benzimidazol-5-yl}methanol

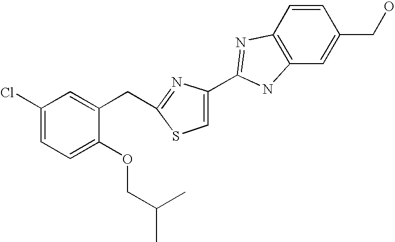

Methyl 2-[2-({5-chloro-2-[(2-methylpropyl)oxy]phenyl}methyl)-1,3-thiazol-4-yl]-1H-benzimidazole-5-carboxylate (0.35 mmol) was dissolved in THF, cooled at −10° C. under argon, 1M LiAlH₄ in THF (0.35 ml) was added, allowed to warm to room temperature. More LiAlH₄ (0.35 ml) was added, the mixture stirred for other 2 h, then quenched with water and extracted with diethyl ether (×4). The combined organic phases were dried (MgSO₄), evaporated and purified on a Biotage using 2-4% of methanol in dichloromethane.

LC/MS Rt=2.62 min, [MH⁺] 428.2, 430.2, [MH⁻] 426.1, 428.

### 2-[2-({5-Chloro-2-[(2-methylpropyl)oxy]phenyl}methyl)-1,3-thiazol-4-yl]-1H-benzimidazole-5-carbaldehyde

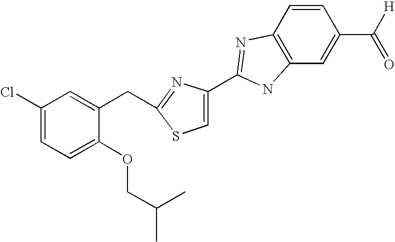

The title compound was prepared in a similar manner to 2-({5-chloro-2-[(phenylmethyl)oxy]phenyl}methyl)-N-(4-formylphenyl)-1,3-thiazole-4-carboxamide.

LC/MS Rt=3.47 min, [MH⁺] 426.2, 428.1 [MH⁻] 424

### {2-[2-({5-Chloro-2-[(2-methylpropyl)oxy]phenyl}methyl)-1,3-oxazol-4-yl]-1H-benzimidazol-5-yl}acetaldehyde

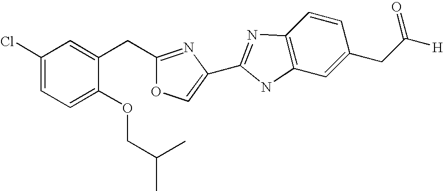

2-{2-[2-({5-Chloro-2-[(2-methylpropyl)oxy]phenyl}methyl)-1,3-oxazol-4-yl]-1H-benzimidazol-5-yl}ethanol (353 mg, 0.829 mmol) was dissolved in THF (6 ml). Dess Martin periodinane (352 mg, 0.829 mmol) was added and the mixture was stirred at room temperature for 1 hour. Diluted with DCM and washed with 10% aqueous sodium thiosulphate followed by saturated sodium hydrogen carbonate. The organic phase was dried and evaporated to give the title compound as a yellow solid (345 mg). LC/MS Rt=3.0, [MH⁺] 424.1, 426.1.

### 2-[2-({5-Chloro-2-[(phenylmethyl)oxy]phenyl}methyl)-1,3-oxazol-4-yl]-1H-benzimidazole-5-carbaldehyde

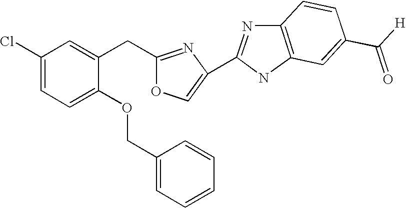

The title compound was prepared in a similar manner to {2-[2-({5-chloro-2-[(2-methylpropyl)oxy]phenyl}methyl)-1,3-oxazol-4-yl]-1H-benzimidazol-5-yl}acetaldehyde.

LC/MS Rt=3.28, [MH⁺] 444.1, 446.1

### 2-[2-({5-Chloro-2-[(2-methylpropyl)oxy]phenyl}methyl)-1,3-oxazol-4-yl]-1H-benzimidazole-5-carbaldehyde

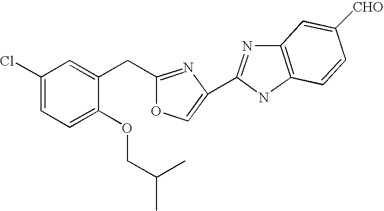

Prepared in a similar manner to {2-[2-({5-chloro-2-[(2-methylpropyl)oxy]phenyl}methyl)-1,3-oxazol-4-yl]-1H-benzimidazol-5-yl}acetaldehyde using {2-[2-({5-chloro-2-[(2-methylpropyl)oxy]phenyl}methyl)-1,3-oxazol-4-yl]-1H-benzimidazol-5-yl}methanol.

LC/MS Rt=3.32, [MH⁺] 410.1, 412.1

### Example 142

### 2-[2-({5-Chloro-2-[(2-methylpropyl)oxy]phenyl}methyl)-1,3-thiazol-4-yl]-5-(4-methyl-1-piperazinyl)-1H-benzimidazole hydrochloride

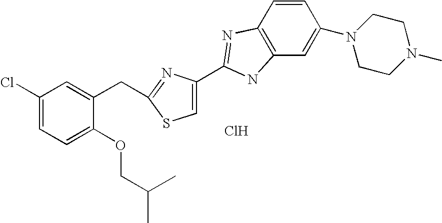

The title compound was prepared in a similar manner to 2-{2-[{5-chloro-2-[(2-methylpropyl)oxy]phenyl}(difluoro)methyl]-1,3-thiazol-4-yl}-1H-benzimidazole hydrochloride. LC/MS Rt=2.14 min, [MH⁺] 496.1 [MH⁻] 494.2, 496.3, 497.3.

### Methyl 2-[2-({5-chloro-2-[(2-methylpropyl)oxy]phenyl}methyl)-1,3-thiazol-4-yl]-1H-benzimidazole-5-carboxylate

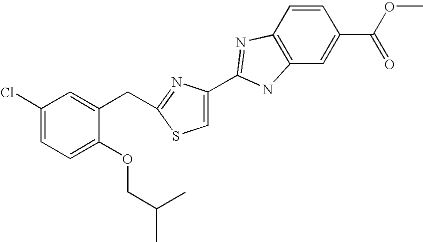

The title compound was prepared in a similar manner to 2-{2-[{5-chloro-2-[(2-methylpropyl)oxy]phenyl}(difluoro)methyl]-1,3-thiazol-4-yl}-1H-benzimidazole hydrochloride using 1.2 equivalent of the methyl 3,4-diaminobenzoate.

LC/MS Rt=3.58 min, [MH⁺] 456.1, 458, [MH⁻] 454.1, 456.1

### Example 143

### {2-[2-({5-Chloro-2-[(phenylmethyl)oxy]phenyl}methyl)-1,3-thiazol-4-yl]-1H-benzimidazol-4-yl}methanol hydrochloride

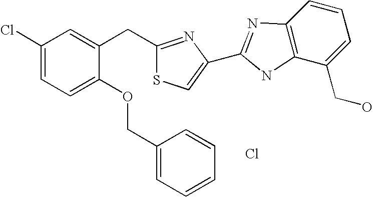

A solution of methyl 2-[2-({5-chloro-2-[(phenylmethyl)oxy]phenyl}methyl)-1,3-thiazol-4-yl]-1H-benzimidazole-4-carboxylate hydrochloride (200 mg, 0.41 mmol) in dry tetrahydrofuran (5 ml) was treated with 1.0M lithium aluminium hydride in THF (0.45 ml, 0.45 mmol) and stirred at room temperature for 1 hour. The reaction was quenched by the careful addition of 2M sodium hydroxide solution. The mixture was extracted with ethyl acetate (3×5 ml). The combined extracts were washed with water, dried and evaporated. Trituration of the residue with diethyl ether gave the title compound as an off-white solid 160 mg 84%.

LC/MS Rt=2.60 min., [MH⁺] 462.

### {2-[2-({5-Chloro-2-[(2-methylpropyl)oxy]phenyl}methyl)-1,3-thiazol-4-yl]-1-1H-benzimidazol-5-yl}methanol

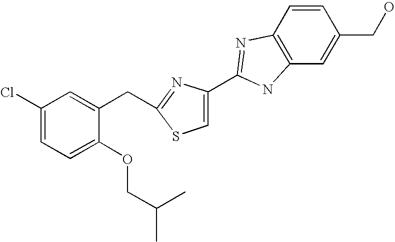

Methyl 2-[2-({5-chloro-2-[(2-methylpropyl)oxy]phenyl}methyl)-1,3-thiazol-4-yl]-1H-benzimidazole-5-carboxylate (0.35 mmol) was dissolved In THF, cooled at −10° C. under argon, 1M LiAlH₄ in THF (0.35 ml) was added, allowed to warm to room temperature. More LiAlH₄ (0.35 ml) was added, the mixture stirred for another 2 h, then quenched with water and extracted with diethyl ether (×4). The combined organic phases were dried (MgSO₄), evaporated and purified on a Biotage using 2-4% of methanol in dichloromethane.

LC/MS Rt=2.62 min, [MH⁺] 428.2, 430.2, [MH⁻] 426.1, 428

### 2-[2-({5-Chloro-2-[(phenylmethyl)oxy]phenyl}methyl)-1,3-thiazol-4-yl]-1H-benzimidazole-4-carbaldehyde

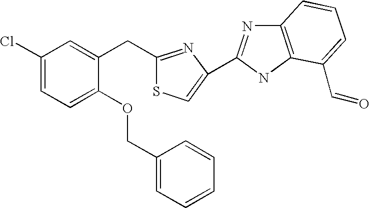

Dess-Martin periodinane (148 mg, 0.35 mmol) was added, under argon, to a stirred solution of {2-[2-({5-chloro-2-[(phenylmethyl)oxy]phenyl}methyl)-1,3-thiazol-4-yl]-1H-benzimidazol-4-yl}methanol (160 mg, 0.35 mmol) in dry dichloromethane (10 ml). The reaction mixture was stirred at room temperature for 2 hours. Saturated aqueous sodium hydrogen carbonate (5 ml) and 10% aq. sodium thiosulphate (5 ml) were added. The organic phase was separated dried and evaporated to give the title compound as a brown gum.

LC/MS Rt=3.51 min., [MH⁺] 460.

### {2-[2-({5-Chloro-2-[(phenylmethyl)oxy]phenyl}methyl)-1,3-thiazol-4-yl]-1H-benzimidazol-5-yl}acetaldehyde

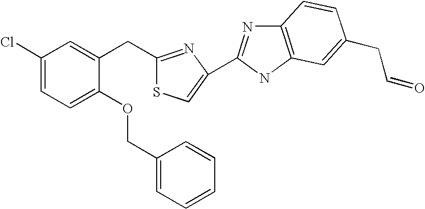

The title compound was prepared in a similar manner to 2-[2-({5-chloro-2-[(phenylmethyl)oxy]phenyl}methyl)-1,3-thiazol-4-yl]-1H-benzimidazole-4-carbaldehyde using 2-{2-[2-({5-chloro-2-[(phenylmethyl)oxy]phenyl}methyl)-1,3-thiazol-4-yl]-1H-benzimidazol-5-yl}ethanol. Crude product used without purification.

### {2-[2-({5-Chloro-2-[(2-methylpropyl)oxy]phenyl}methyl)-1,3-thiazol-4-yl]-1H-benzimidazol-5-yl}acetaldehyde

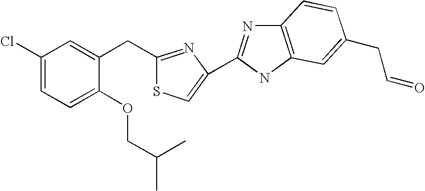

The title compound was prepared in a similar manner to 2-[2-({5-chloro-2-[(phenylmethyl)oxy]phenyl}methyl)-1,3-thiazol-4-yl]-1H-benzimidazole-4-carbaldehyde using 2-{2-[2-({5-chloro-2-[(2-methylpropyl)oxy]phenyl}methyl)-1,3-thiazol-4-yl]-1H-benzimidazol-5-yl}ethanol. Crude product used without purification.

### Example 144

### 2-[2-({5-Chloro-2-[(2-methylpropyl)oxy]phenyl}methyl)-1,3-thiazol-4-yl]-5-(4-morpholinyl)-1H-imidazo[4,5-b]pyridine

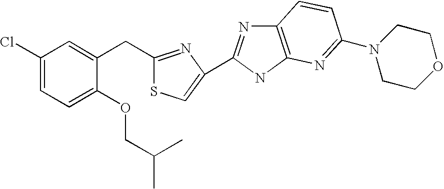

A solution of 5-chloro-2-[2-({5-chloro-2-[(2-methylpropyl)oxy]phenyl}methyl)-1,3-thiazol-4-yl]-1H-imidazo[4,5-b]pyridine (50 mg, 0.1 mmol), morpholine (34 mg, 0.4 mmol), and methanesuphonic acid (38 mg, 0.4 mmol) in 1,4-dioxane (1 ml) was heated, in a microwave, to 180° C. for 11 hours. The reaction mixture was cooled to room temperature and diluted with methanol (5 ml). The solvent was evaporated and the residue dissolved in ethyl acetate (10 ml). The solution was washed with saturated sodium hydrogen carbonate, water and brine. The organic phase was dried and evaporated and the residue purified by MDAP to give the title compound as a colourless solid.

LC/MS Rt=3.07 min, [MH⁺] 484.

The following compounds were prepared in a similar manner to 2-[2-({5-Chloro-2-[(2-methylpropyl)oxy]phenyl}methyl)-1,3-thiazol-4-yl]-5-(4-morpholinyl)-1H-imidazo[4,5-b]pyridine using the appropriate amine.

| Example | | Name | Data |
|---|---|---|---|
| 145 | 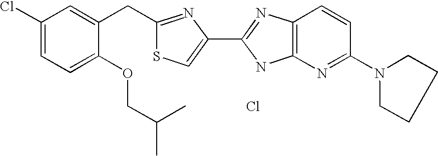 | 2-[2-({5-Chloro-2-[(2-methylpropyl)oxy]phenyl}methyl)-1,3-thiazol-4-yl]-5-(1-pyrrolidinyl)-1H-imidazo[4,5-b]pyridinehydrochloride | LC/MS Rt =2.69 min,[MH⁺] 468. |
| 146 | 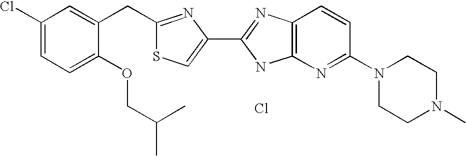 | 2-[2-({5-chloro-2-[(2-methylpropyl)oxy]phenyl}methyl)-1,3-thiazol-4-yl]-5-(4-methyl-1-piperazinyl)-1H-imidazo[4,5-b]pyridinehydrochloride | LC/MS Rt =2.21 min,[MH⁻] 495. |
| 147 | 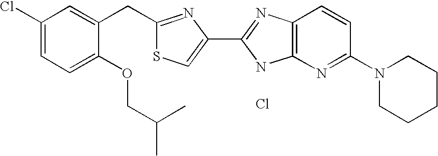 | 2-({5-Chloro-2-[(2-methylpropyl)oxy]phenyl}methyl)-1,3-thiazol-4-yl]-5-(1-piperidinyl)-1H-imidazo[4,5-b]pyridinehydrochloride | LC/MS Rt =3.24 min,[MH⁺] 482. |

### Example 148

### 2-[2-({5-Chloro-2-[(phenylmethyl)oxy]phenyl}methyl)-1,3-thiazol-4-yl]-5-(4-methyl-1-piperazinyl)-1H-benzimidazole hydrochloride

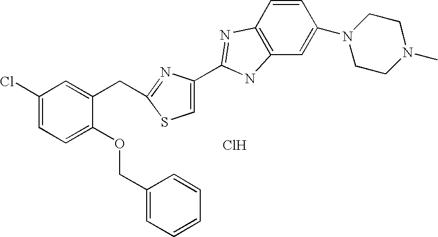

Methyl 2-({5-chloro-2-[(phenylmethyl)oxy]phenyl}methyl)-1,3-thiazole-4-carboximidoate hydrochloride (200 mg, 0.49 mmol) was dissolved in EtOH (4 ml) and 4-(4-methyl-1-piperazinyl)-1,2-benzenediamine (59 mg, 0.54 mmol) was added and the mixture heated at 90° C. for 3 h. The solvent was evaporated and the residue was diluted with water, basified with 2M NaOH and extracted with ethyl acetate (×5). The combined organic extracts were dried (MgSO₄) and evaporated. The residue was purified on a Biotage using DCMWM/eOH gradient mixture to give a solid. The solid was treated with 1M HCl in Et₂O to give the title compound. LC/MS Rt=2.06 min, [MH⁺] 530.1, 533.2 [MH] 528.2, 530.2.

The following compounds were prepared in a manner similar to 2-[2-({5-chloro-2-[(phenylmethyl)oxy]phenyl}methyl)-1,3-thiazol-4-yl]-5-(4-methyl-1-piperazinyl)-1H-benzimidazole hydrochloride

| Example | | Name | Data |
|---|---|---|---|
| 149 | 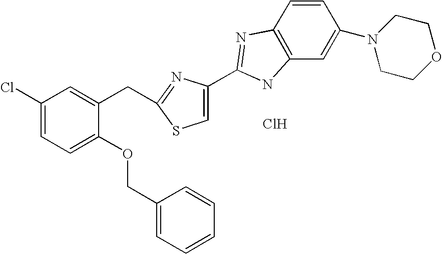 | 2-[2-({5-Chloro-2-[(phenylmethyl)oxy]phenyl}methyl)-1,3-thiazol-4-yl]-5-(4-morpholinyl)-1H-benzimidazole hydrochloride | LC/MS Rt =2.65 min,[MH⁺] 517.1,519.1, 521.1[MH⁻] 515.2,517.1 |
| 150 | 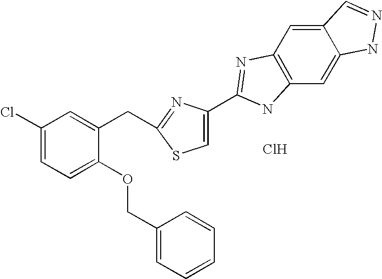 | 6-[2-({5-Chloro-2-[(phenylmethyl)oxy]phenyl}methyl)-1,3-thiazol-4-yl]-1,5-dihydroimidazo[4,5-f]indazolehydrochloride | LC/MS Rt =2.64 min,[MH⁺] 472,474 [MH⁻]470.1, 472.1 |

### Example 151

### 2-[2-({5-Chloro-2-[(phenylmethyl)oxy]phenyl}methyl)-1,3-thiazol-4-yl]-5-(4-methyl-1-piperazinyl)-1H-imidazo[4,5-b]pyridine hydrochloride

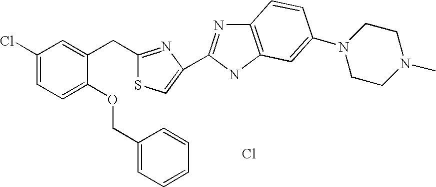

A solution of 5-chloro-2-[2-({5-chloro-2-[(phenylmethyl)oxy]phenyl}methyl)-1,3-thiazol-4-yl]-1H-imidazo[4,5-b]pyridine hydrochloride (100 mg, 0.21 mmol) in 1-methyl-2-pyrrolidinone (2 ml) was treated with 1-methylpiperazine (0.5 ml, large excess) and water (a few drops). The mixture was heated in a microwave at 200° C. for 6 hours. The mixture was partitioned between ethyl acetate (25 ml) and water (25 ml). The aqueous phase was extracted with ethyl acetate (20 ml). The combined organics were washed with water (3×10 ml), dried and evaporated. Purification of the residue by flash chromatography eluting with 5% methanol in dichloromethane followed by conversion to the hydrochloride salt by treatment with hydrogen chloride in diethyl ether gave the title compound as a pale yellow solid 20 mg LC/MS Rt=2.29 min., [MH⁺] 531.

### Example 152

### 2-[2-({-5-Chloro-2-[(phenylmethyl)oxy]phenyl}methyl)-1,3-thiazol-4-yl]-N,N-dimethyl-1H-imidazo[4,5-b]pyridin-5-amine hydrochloride

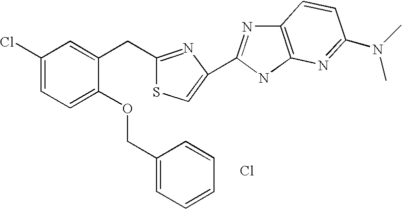

The title compound was prepared in a similar manner to 2-[2-({5-Chloro-2-[(phenylmethyl)oxy]phenyl}methyl)-1,3-thiazol-4-yl]-5-(4-methyl-1-piperazinyl)-1H-imidazo[4,5-b]pyridine hydrochloride using 5-chloro-2-[2-({5-chloro-2-[(phenylmethyl)oxy]phenyl}methyl)-1,3-thiazol-4-yl]-1H-imidazo[4,5-b]pyridine and 40% aqueous dimethylamine. LC/MS Rt=2.70 min., [MH⁺] 476.

### Example 153

### 2-{2-[2-({5-Chloro-2-[(phenylmethyl)oxy]phenyl}methyl)-1,3-thiazol-4-yl]-1H-benzimidazol-5-yl}ethanol

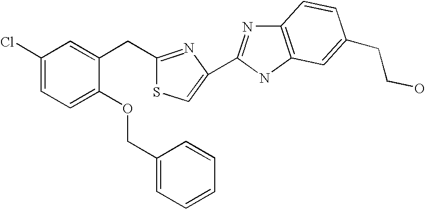

A mixture of methyl 2-({5-chloro-2-[(phenylmethyl)oxy]phenyl}methyl)-1,3-thiazole-4-carboximidoate hydrochloride (450 mg, 1.1 mmol) and 2-(3,4-diaminophenyl)ethanol (182 mg, 1.2 mmol) in ethanol (10 ml) was refluxed for 1 hour. The mixture was cooled to room temperature and the solvent evaporated. Purification of the residue by flash chromatography eluting with 30-60% ethyl acetate in hexane followed by 5% methanol in dichloromethane gave the title compound as a pale yellow solid 180 mg 34%. LC/MS Rt=2.51 min., [MH⁺] 476.

### Example 154

### 2-{2-[2-({5-Chloro-2-[(2-methylpropyl)oxy]phenyl}methyl)-1,3-thiazol-4-yl]-1H-benzimidazol-5-yl}ethanol

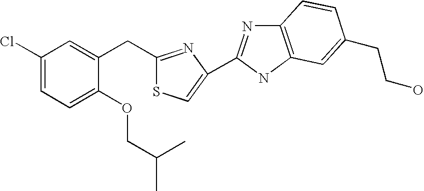

A solution of N-[2-amino-5-(2-hydroxyethyl)phenyl]-2-({5-chloro-2-[(2-methylpropyl)oxy]phenyl}methyl)-1,3-thiazole-4-carboxamide (660 mg, 1.44 mmol) in glacial acetic acid (5 ml) was refluxed for 2 hours. The mixture was cooled, diluted with water (20 ml) and extracted with ethyl acetate (2×10 ml). The combined extracts were washed with saturated sodium hydrogen carbonate, water and brine. The organic phase was dried and evaporated. Purification of the residue by flash chromatography eluting with 30-60% ethyl acetate in hexane followed by 5% methanol in dichloromethane gave the title compound as a pale yellow solid 100 mg. LC/MS Rt=2.53 min., [MH⁺] 442.

### 2-{2-[2-({5-Chloro-2-[(2-methylpropyl)oxy]phenyl}methyl)-1,3-oxazol-4-yl]-1H-benzimidazol-5-yl}ethanol

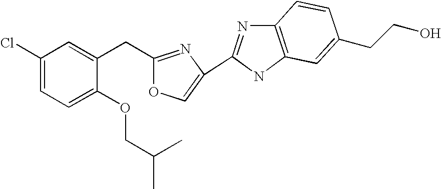

2-({5-Chloro-2-[(2-methylpropyl)oxy]phenyl}methyl)-1,3-oxazole-4-carboxylic acid (900 mg, 2.9 mmol), EDAC (666 mg, 3.49 mmol), HOBt (471 mg, 3.49 mmol), 4-methylmorpholine (5.8 mmol, 638 μl) and 2-(3,4-diaminophenyl)ethanol (530 mg, 3.49 mmol) were stirred at room temperature in dichloromethane (30 ml) for 3 hours. Diluted with more DCM and washed with a saturated solution of sodium bicarbonate and water. The organic phase was dried and evaporated to give a dark oil that was purified on a 50 g SPE Si column with 5% of methanol in ethyl acetate to give a yellow oil (530 mg).

The yellow oil (530 mg) was dissolved in 5 ml of acetic acid and heated at 110° C. for 30 minutes. The mixture was then diluted with water, extracted with EtOAc (×3), the combined organics were washed with saturated sodium bicarbonate solution (×3), dried (MgSO₄) and evaporated.

The residue was chromatographed using a mixture of hexane/ethyl acetate to give the title compound as a yellow solid (157 mg).

LC/MS Rt=2.61, [MH⁺] 426.2, 428.2, [MH⁻] 1424.2, 426.2

### Methyl 2-[2-({5-chloro-2-[(phenylmethyl)oxy]phenyl}methyl)-1,3-oxazol-4-yl]-1H-benzimidazole-5-carboxylate

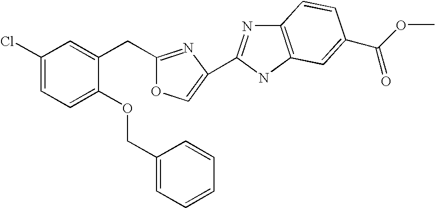

The title compound was prepared in a similar manner to 2-{2-[2-({5-chloro-2-[(2-methylpropyl)oxy]phenyl}methyl)-1,3-oxazol-4-yl]-1H-benzimidazol-5-yl}ethanol.

LC/MS Rt=3.41, [MH⁺] 474.2, 476.2

### {2-[2-({5-Chloro-2-[(phenylmethyl)oxy]phenyl}methyl)-1,3-oxazol-4-yl]-1H-benzimidazol-5-yl}methanol

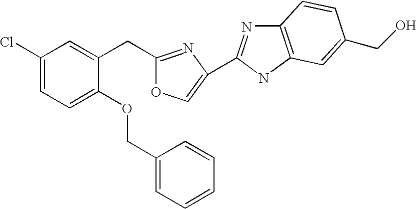

Methyl 2-[2-({5-chloro-2-[(phenylmethyl)oxy]phenyl}methyl)-1,3-oxazol-4-yl]-1H-benzimidazole-5-carboxylate (648 mg, 1.36 mmol in THF (8 ml) under argon was cooled to −10° C., 1.0M lithium aluminium hydride in THF (1.5 ml) was added. The reaction mixture was warmed to room temperature, more LiAlH₄ (0.35 ml) was added and solution stirred for another 40 minutes. The mixture was quenched with water, diluted with diethyl ether, filtered off insoluble solid on celite, aqueous extracted with ethyl ether (×3), dried and evaporated. The residue was purified on an SPE column using hexane/ethyl acetate gradient to give the title compound as an orange solid (400 mg).

LC/MS Rt=2.57, [MH⁺] 446.1, 448.1

### {2-[2-({5-Chloro-2-[(2-methylpropyl)oxy]phenyl}methyl)-1,3-oxazol-4-yl]-1H-benzimidazol-5-yl}methanol

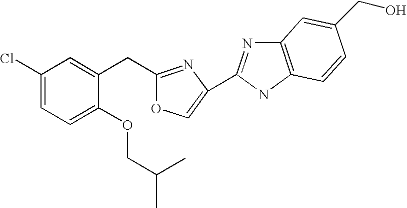

Was prepared in a similar manner to {2-[2-({5-chloro-2-[(phenylmethyl)oxy]phenyl}methyl)-1,3-oxazol-4-yl]-1H-benzimidazol-5-yl}methanol using methyl 2-[2-({5-chloro-2-[(2-methylpropyl)oxy]phenyl}methyl)-1,3-oxazol-4-yl]-1H-benzimidazole-5-carboxylate as starting material. LC/MS Rt=2.61, [MH+] 412.2, 414.3

### N-[2-Amino-5-(2-hydroxyethyl)phenyl]-2-({5-chloro-2-[(2-methylpropyl)oxy]phenyl}methyl)-1,3-thiazole-4-carboxamide

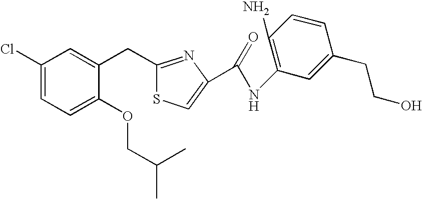

The title compound was prepared in a similar manner to 2-({5-chloro-2-[(phenylmethyl)oxy]phenyl}methyl)-N-phenyl-1,3-thiazole-4-carboxamide using 2-({5-chloro-2-[(2-methylpropyl)oxy]phenyl}methyl)-1,3-thiazole-4-carboxylic acid and 2-(3,4-diaminophenyl)ethanol. LC/MS Rt=3.23 min., [MH⁺] 460.

### Example 155

### 2-[2-({5-Chloro-2-[(2-methylpropyl)oxy]phenyl}methyl)-1,3-thiazol-4-yl]-5,6-difluoro-1H-benzimidazole

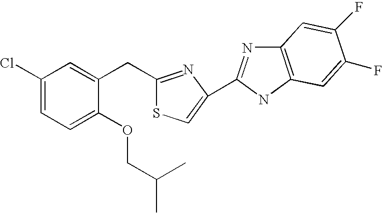

A mixture of ethyl 2-({5-chloro-2-[(2-methylpropyl)oxy]phenyl}methyl)-1,3-thiazole-4-carboximidoate hydrochloride (100 mg, 0.26 mmol) and 4,5-difluoro-1,2-phenylenediamine (45 mg, 0.3 mmol) in ethanol (5 ml) was heated at 80° C. for 2 hours. The reaction mixture was cooled to room temperature and partitioned between ethyl acetate and water. The organic phase was separated, dried and evaporated. Purification by column chromatography gave the title compound. LC/MS Rt=3.62 min, [MH⁺] 434.

### 2-({5-Chloro-2-[(phenylmethyl)oxy]phenyl}methyl)-N-[(1E)-(dimethylamino)methylidene]-1,3-thiazole-4-carboxamide

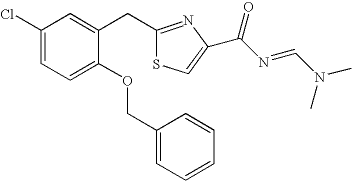

A solution of 2-({5-chloro-2-[(phenylmethyl)oxy]phenyl}methyl)-1,3-thiazole-4-carboxamide (500 mg, 1.4 mmol) in dimethylformamide dimethyl acetal (2 ml) was heated at 120° C. for two hours. The reaction mixture was allowed to cool to room temperature. The title compound crystallised and was collected by filtration 400 mg, 70%.

¹H NMR (CDCl₃) δ: 3.18 (3H, s), 3.19 (3H, s), 4.43 (2H, s) 5.09 (2H, s), 6.86 (1H, d), 7.12-7.76 (7H, m), 8.15 (1H, s), 8.67 (1H, s).

### 2-({5-Chloro-2-[(2-methylpropyl)oxy]phenyl}methyl)-N-[(1E)-(dimethylamino)methylidene]-1,3-thiazole-4-carboxamide

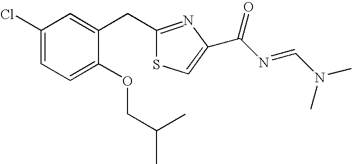

The title compound was prepared in a similar manner to 2-({5-chloro-2-[(phenylmethyl)oxy]phenyl}methyl)-N-[(1E)-(dimethylamino)methylidene]-1,3-thiazole-4-carboxamide using 2-({5-chloro-2-[(2-methylpropyl)oxy]phenyl}methyl)-1,3-thiazole-4-carboxamide.

¹H NMR (CDCl3) δ: 0.96 (6H, d), 2.03-2.09 (1H, m), 3.18 (3H, s), 3.20 (3H, s), 3.71 (2H, d), 4.39 (2H, s), 6.79 (1H, s), 7.18-7.24 (2H, m), 8.15 (1H, s), 8.67 (1H, s).

### 2-({5-Chloro-2-[(2-methylpropyl)oxy]phenyl}methyl)-N-[(1E)-1-(dimethylamino)ethylidene]-1,3-thiazole-4-carboxamide

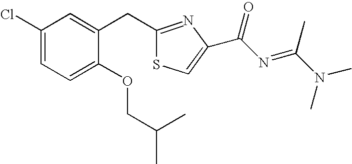

The title compound was prepared in a similar manner to 2-({5-chloro-2-[(phenylmethyl)oxy]phenyl}methyl)-N-[(1E)-(dimethylamino)methylidene]-1,3-thiazole-4-carboxamide using 2-({5-chloro-2-[(2-methylpropyl)oxy]phenyl}methyl)-1,3-thiazole-4-carboxamide and dimethylacetamide dimethylacetal. The product was used directly without purification.

### 2-({5-Chloro-2-[(phenylmethyl)oxy]phenyl}methyl)-N-[(1E)-1-(dimethylamino)ethylidene]-1,3-thiazole-4-carboxamide

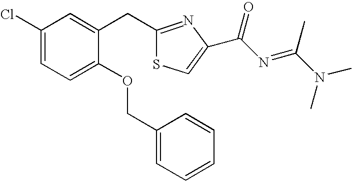

The title compound was prepared in a similar manner to 2-({5-chloro-2-[(phenylmethyl)oxy]phenyl}methyl)-N-[(1E)-(dimethylamino)methylidene]-1,3-thiazole-4-carboxamide using dimethylacetamide dimethylacetal. The product was used directly without purification.

### Example 156

### 3-[2-({5-Chloro-2-[(phenylmethyl)oxy]phenyl}methyl)-1,3-thiazol-4-yl]-1H-1,2,4-triazole

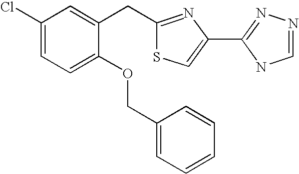

A mixture of hydrazine hydrate (18 mg, 0.35 mmol) and 2-({5-chloro-2-[(phenylmethyl)oxy]phenyl}methyl)-N-[(1E)-(dimethylamino)methylidene]-1,3-thiazole-4-carboxamide (133 mg, 0.32 mmol) in glacial acetic acid (1 ml) was refluxed for 1 hour. The reaction mixture was cooled to room temperature, the solvent was evaporated and the residue triturated with ethyl acetate/hexane to give the title compound as a pale yellow solid 70 mg, 57%. LC/MS Rt=2.90 min, [MH⁺] 383.

The following examples were prepared in a similar manner to 3-[2-({5-chloro-2-[(phenylmethyl)oxy]phenyl}methyl)-1,3-thiazol-4-yl]-1H-1,2,4-triazole from the appropriate intermediates.

| Example | | Name | Data |
|---|---|---|---|
| 157 | 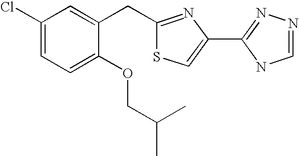 | 3-[2-({5-Chloro-2-[(2-methyl-propyl)oxy]-phenyl}methyl)-1,3-thiazol-4-yl]-1H-1,2,4-triazole | LC/MS Rt =2.96 min,[MH⁺] 349. |
| 158 | 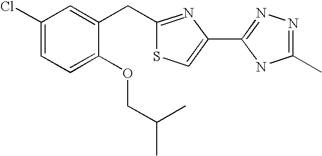 | 3-[2-({5-Chloro-2-[(2-methyl-propyl)oxy]-phenyl}methyl)-1,3-thiazol-4-yl]-5-methyl-1H-1,2,4-triazole | LC/MS Rt =2.95 min,[MH⁺] 363. |
| 159 | 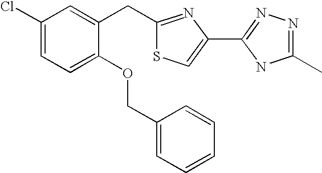 | 3-[2-({5-Chloro-2-[(phenylmethyl)oxy]phenyl}methyl)-1,3-thiazol-4-yl]-5-methyl-1H-1,2,4-triazole | LC/MS Rt =2.89 min,[MH⁺] 397. |

### 2-({5-Chloro-2-[(phenylmethyl)oxy]phenyl}methyl)-N-methyl-N-(methyloxy)-1,3-thiazole-4-carboxamide

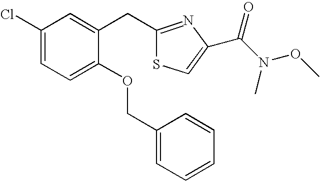

2-({5-Chloro-2-[(phenylmethyl)oxy]phenyl}methyl)-1,3-thiazole-4-carboxylic acid (360 mg, 1.0 mmol) was dissolved in dichloromethane (5 ml) and N-methylmorpholine (202 mg, 2 mmol), N-hydroxybenzotriazole hydrate (184 mg, 1.2 mmol), N-(3-dimethylaminopropyl)-N′-ethylcarbodiimide (230 mg, 1.2 mmol) and O,N-dimethylhydroxylamine hydrochloride (116 mg, 1.2 mmol) were added. The reaction mixture was stirred at room temperature overnight. Then the mixture was diluted with ethyl acetate (20 ml) and washed with saturated sodium hydrogen carbonate, 2M hydrochloric acid, water and brine. The organic phase was dried and evaporated. Purification of the residue by chromatography eluting with 20% ethyl acetate in hexane gave the title compound as a colourless solid 340 mg, 80%. LC/MS Rt=3.22 min, [MH⁺] 403.

### 1-[2-({5-Chloro-2-[(phenylmethyl)oxy]phenyl}methyl)-1,3-thiazol-4-yl]ethanone

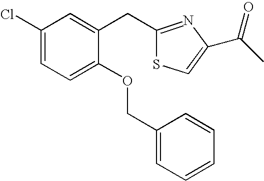

A solution of 2-({5-chloro-2-[(phenylmethyl)oxy]phenyl}methyl)-N-methyl-N-(methyloxy)-1,3-thiazole-4-carboxamide (340 mg, 0.84 mmol) in dry tetrahydrofuran (5 ml) was cooled to 0° C. and treated with 3.0M methylmagnesium bromide in diethyl ether (0.330 ml, 0.9 mmol). The mixture was stirred at 0° C. for 30 minutes, then diluted with diethyl ether (10 ml). The solution was washed with 2M hydrochloric, and water. The organic phase was dried and evaporated to give the title compound as an off-white solid 250 mg 83%.

LC/MS Rt=3.37 min, [MH⁺] 358.

### 2-Bromo-1-[2-({5-chloro-2-[(phenylmethyl)oxy]phenyl}methyl)-1,3-thiazol-4-yl]ethanone

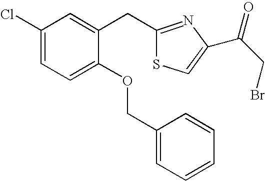

Bromine (112 mg, 0.7 mmol) was added to a solution of 1-[2-({5-chloro-2-[(phenylmethyl)oxy]phenyl}methyl)-1,3-thiazol-4-yl]ethanone (250 mg, 0.7 mmol) in chloroform (5 ml). The mixture was refluxed for 1 hour, a further portion of bromine (112 mg, 0.7 mmol) was added and reflux continued for another hour. The reaction mixture was cooled to room temperature, the solvent was evaporated and the residue chromatographed eluting with 5% ethyl acetate in hexane to give the title compound as a colourless oil 130 mg 43%. LC/MS Rt=3.61 min. [MH⁺] 436, 438.

### Example 160

### 2-[2-({5-Chloro-2-[(phenylmethyl)oxy]phenyl}methyl)-1,3-thiazol-4-yl]imidazo[1,2-a]pyridine

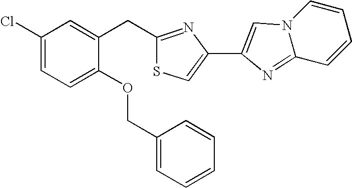

A mixture of 2-bromo-1-[2-({5-chloro-2-[(phenylmethyl)oxy]phenyl}methyl)-1,3-thiazol-4-yl]ethanone (130 mg, 0.3 mmol) and 2-aminopyridine (56 mg, 0.6 mmol) in ethanol (5 ml) was refluxed for 1 hour. The mixture was cooled and the solvent evaporated. The residue was partitioned between ethyl acetate and water. The organic phase was separated dried and evaporated. Trituration of the residue with diethyl ether gave the title compound as an off-white soild 70 mg 54%. LC/MS Rt=2.65 min, [MH⁺] 432.

### Example 161

### 1,1-Dimethylethyl [2-({5-chloro-2-[(phenylmethyl)oxy]phenyl}methyl)-1,3-oxazol-4-yl]carbamate

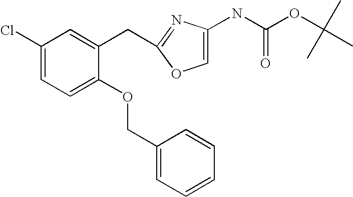

Sodium 2-({5-chloro-2-[(phenylmethyl)oxy]phenyl}methyl)-1,3-oxazole-4-carboxylate was suspended in water and acidified with 2M hydrochloric acid and extracted with ethyl acetate (×3), the combined organic phases were dried, filtered and evaporated to give the free acid, 150 mg. 2-({5-Chloro-2-[(phenylmethyl)oxy]phenyl}methyl)-1,3-oxazole-4-carboxylic acid (150 mg, 0.43 mmol) was dissolved in t-butanol (5 ml), triethylamine (73 μl, 0.52 mmol) and diphenyl phosphorylazide (104 μl, 0.48 mmol) were added and the resulting solution was refluxed for 5 hours. After cooling the mixture was evaporated and the residue was purified by flash chromatography with 10% of ethyl acetate in iso-hexane to yield the title compound as a white solid.

¹H NMR (CDCl₃)δ: 1.49 (9H, s), 4.04 (2H, s), 5.05 (2H, s), 6.69 (1H, bs), 6.84 (1H, d), 7.17-7.23 (2H, m), 7.30-7.40 (5H, m), 7.67 (1H, bs).

### Example 162

### 2-[2-({5-Chloro-2-[(2-methylpropyl)oxy]phenyl}methyl)-1,3-oxazol-4-yl]-1H-benzimidazole hydrochloride

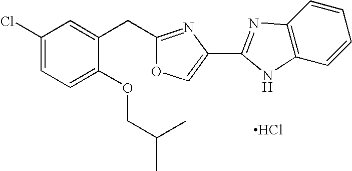

A mixture of methyl 2-({5-chloro-2-[(2-methylpropyl)oxy]phenyl}methyl)-1,3-oxazole-4-carboximidoate hydrochloride (72 mg, 0.2 mmol) and 1,2-phenylenediamine (27 mg, 0.25 mmol) in methanol (4 ml) was stirred and refluxed for 4 hours. The resulting solution was cooled, diluted with ether/water and basified with 2M sodium hydroxide. The organic phase was dried with magnesium sulphate, evaporated and purified by chromatography on a Biotage eluting with (1:3) ethyl acetate/hexane. The product was dissolved in dichloromethane and 1M hydrogen chloride in ether (1 ml) was added. The solution was evaporated to dryness to give the title compound as a solid (70 mg).

LC/MS Rt=3.00, [MH⁺] 382, 384

The following compounds were prepared in a similar manner to 2-[2-({5-chloro-2-[(2-methylpropyl)oxy]phenyl}methyl)-1,3-oxazol-4-yl]-1H-benzimidazole hydrochloride.

| Example | | Name | Data |
|---|---|---|---|
| 163 | 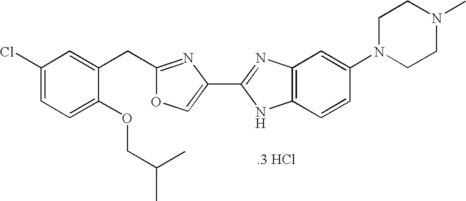 | 2-[2-({5-Chloro-2-[(2-methylpropyl)oxy]phenyl}methyl)-1,3-oxazol-4-yl]-5-(4-methyl-1-piperazinyl)-1H-benzimidazoletrihydrochloride | LC/MS Rt =2.06, [MH−]478.2,480.2 |
| | 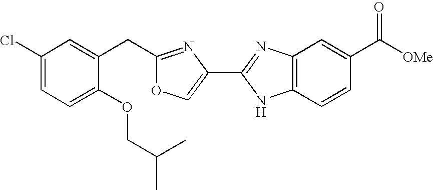 | Methyl 2-[2-({5-chloro-2-[(2-methyl-propyl)oxy]phenyl}methyl)-1,3-oxazol-4-yl]-1H-benzimidazole-5-carboxylate | LC/MS Rt =3.44, (MH+]440.1,442.1 |
| 164 | 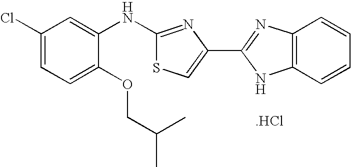 | 4-(1H-Benzimidazol-2-yl)-N-{5-chloro-2-[(2-methylpropyl)oxy]phenyl}-1,3-thiazol-2-aminehydrochloride | LC/MS Rt =2.81 [MH]⁺399.19,401.19 |
| 165 | 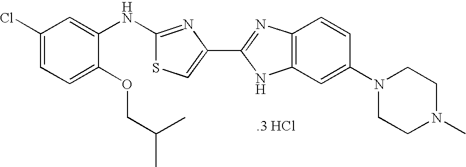 | N-{5-Chloro-2-[(2-methylpropyl)oxy]phenyl}-4-[5-(4-methyl-1-piperazinyl)-1H-benzimidazol-2-yl]-1,3-thiazol-2-aminetrihydrochloride | LC/MS Rt =2.03 [MH]⁻495.21,497.19 |

### Example 166

### (2-{2-[2-({5-Chloro-2-[(2-methylpropyl)oxy]phenyl}methyl)-1,3-oxazol-4-yl]-1H-benzimidazol-5-yl}ethyl)dimethylamine hydrochloride

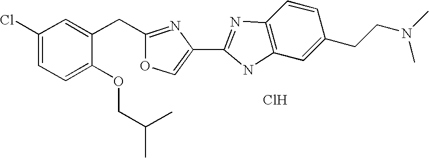

A mixture of {2-[2-({5-chloro-2-[(2-methylpropyl)oxy]phenyl}methyl)-1,3-oxazol-4-yl]-1H-benzimidazol-5-yl}acetaldehyde (86 mg, 0.2 mmol), sodium triacetoxyborohydride (129 mg, 0.6 mmol) and dimethylamine (108 μl, 0.6 mmol, 5.6M in EtOH) in THF (4 ml) was stirred at room temperature over the weekend. Diluted with H₂O and extracted with ethyl acetate (×3), combined organics dried and evaporated. The residue was purified on MDAP, solvent was evaporated, redissolved in methanol, treated with 1M HCl in Et₂O and evaporated to give the title compound. LC/MS Rt=2.24, [MH⁻] 451.2, 453.2

The following examples were prepared in a similar manner to (2-{2-[2-({5-chloro-2-[(2-methylpropyl)oxy]phenyl}methyl)-1,3-oxazol-4-yl]-1H-benzimidazol-5-yl}ethyl)dimethylamine hydrochloride:

| Example | | Name | Data |
|---|---|---|---|
| 167 | 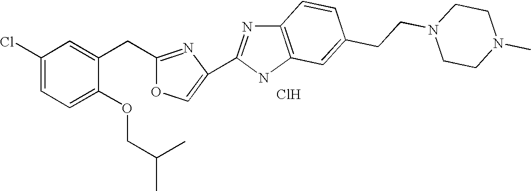 | 2-[2-({5-chloro-2-[(2-methylpropyl)oxy]phenyl}methyl)-1,3-oxazol-4-yl]-5-[2-(4-methyl-1-piperazinyl)ethyl]-1H-benzimidazolehydrochloride | LC/MS Rt =2.1, [MH⁻]506.3, 508.2 |
| 168 | 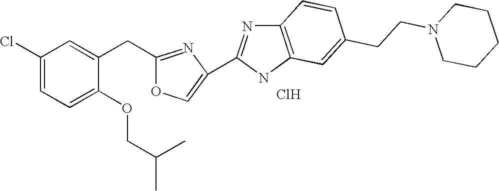 | 2-[2-({5-chloro-2-[(2-methylpropyl)oxy]phenyl}methyl)-1,3-oxazol-4-yl]-5-[2-(1-piperidinyl)ethyl]-1H-benzimidazolehydrochloride | LC/MS Rt =2.19, [MH⁺]493.2,496.2 [MH⁻]491.3, 493.2 |
| 169 | 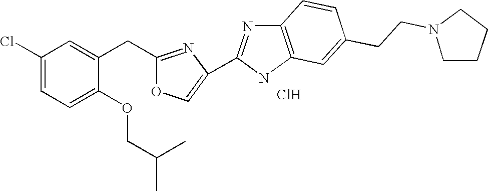 | 2-[2-({5-chloro-2-[(2-methylpropyl)oxy]phenyl}methyl)-1,3-oxazol-4-yl]-5-[2-(1-pyrrolidinyl)ethyl]-1H-benzimidazolehydrochloride | LC/MS Rt =2.19, [MH⁺]479.2, 481.2,482.2 [MH⁻]477.3, 479.2 |
| 170 | 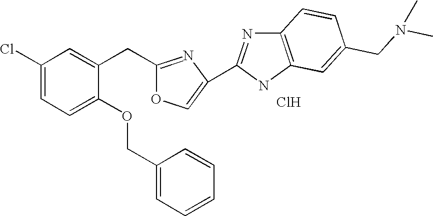 | ({2-[2-({5-chloro-2-[(phenylmethyl)oxy]phenyl}-methyl)-1,3-oxazol-4-yl]-1H-benzimidazol-5-yl}methyl)dimethylaminehydrochloride | LC/MS Rt =2.2, [MH⁻]471.3, 473.2 |
| 171 | 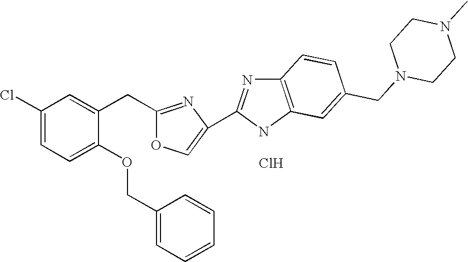 | 2-[2-({5-chloro-2-[(phenylmethyl)oxy]phenyl}-methyl)-1,3-oxazol-4-yl]-5-[(4-methyl-1-piperazinyl)methyl]-1H-benzimidazolehydrochloride | LC/MS Rt =2.1, [MH⁻]526.2, 528.2 |
| 172 | 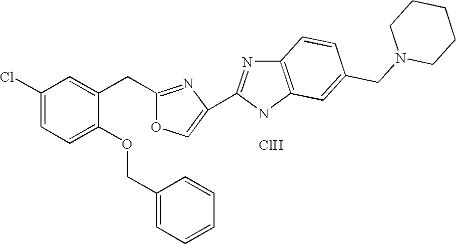 | 2-[2-({5-chloro-2-[(phenylmethyl)oxy]phenyl}-methyl)-1,3-oxazol-4-yl]-5-(1-piperidinylmethyl)-1H-benzimidazolehydrochloride | LC/MS Rt =2.22, [MH⁻]511.3, 513.2 |
| 173 | 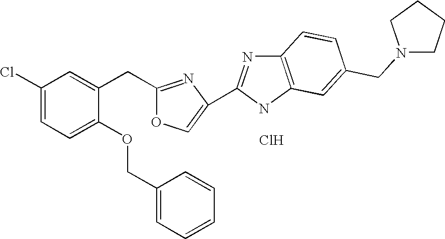 | 2-[2-({5-chloro-2-[(phenylmethyl)oxy]phenyl}-methyl)-1,3-oxazol-4-yl]-5-(1-pyrrolidinylmethyl)-1H-benzimidazolehydrochloride | LC/MS Rt =2.25, [MH⁻]497.2, 499.2 |
| 174 | 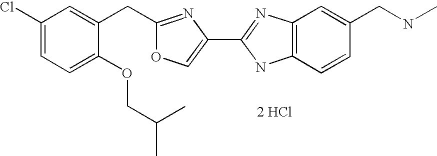 | ({2-[2-({5-Chloro-2-[(2-methylpropyl)oxy]phenyl}methyl)-1,3-oxazol-4-yl]-1H-benzimidazol-5-yl}methyl)methylaminedihydrochloride | LC/MS Rt =2.23, [MH⁻]423.15, 425.11 |
| 175 | 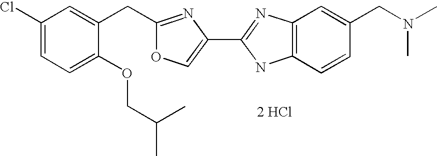 | ({2-[2-({5-Chloro-2-[(2-methylpropyl)oxy]phenyl}methyl)-1,3-oxazol-4-yl]-1H-benzimidazol-5-yl}methyl)dimethylaminedihydrochloride | LC/MS Rt =2.24, [MH⁻]437.17, 439.15 |
| 176 | 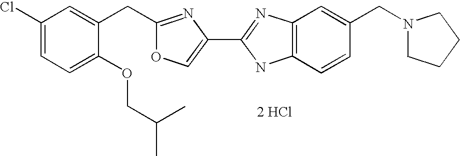 | 2-[2-({5-chloro-2-[(2-methylpropyl)oxy]phenyl}methyl)-1,3-oxazol-4-yl]-5-(1-pyrrolidinylmethyl)-1H-benzimidazoledihydrochloride | LC/MS Rt =2.24, [MH⁻]463.2, 465.2 |
| 177 | 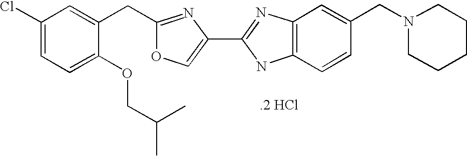 | 2-[2-({5-chloro-2-[(2-methylpropyl)oxy]phenyl}methyl)-1,3-oxazol-4-yl]-5-(1-piperidinylmethyl)-1H-benzimidazoledihydrochloride | LC/MS Rt =2.32, [MH⁻]477.18, 479.16 |
| 178 | 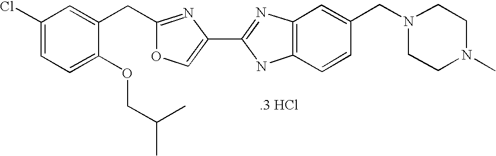 | 2-[2-({5-Chloro-2-[(2-methylpropyl)oxy]phenyl}methyl)-1,3-oxazol-4-yl]-5-[(4-methyl-1-piperazinyl)methyl]-1H-benzimidazoletrihydrochloride | LC/MS Rt =2.08, [MH⁻]492.2, 494.3 |

### Example 179

### N-{5-Chloro-2-[2-methylpropyl)oxy]phenyl}-4-{5-[(methylamino)methyl]-1H-benzimidazol-2-yl}-1,3-thiazol-2-amine hydrochloride

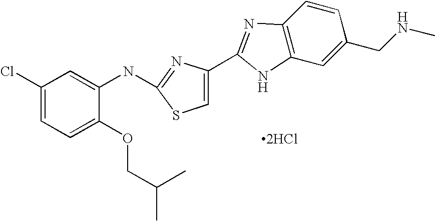

2-[2-({5-Chloro-2-[(2-methylpropyl)oxy]phenyl}amino)-1,3-thiazol-4-yl]-1H-benzimidazole-5-carbaldehyde (90.5 mg, 0.21 mmol) was dissolved in THF (2 ml) and 40 wt % methylamine in water (33 ul, 0.42 mmol) and sodium triacetoxyborohydride (137 mg, 0.63 mmom) added. The reaction was stirred under argon at room temperature for 2 hours. Ethyl acetate and water were added and the organic layer dried (MgSO₄) and evaporated. The residue was purified by flash chromatography, eluting with 2-20% methanol in dichloromethane. The product was dissolved in dichloromethane (1 ml) and 1M HCl in diethyl ether (1 ml) added. The solid was isolated by decantation and dried 32 mg. LC/MS Rt=2.21, [MH⁺] 442, 444.

The following examples were prepared in a similar manner to N-{5-chloro-2-[(2-methylpropyl)oxy]phenyl}-4-{5-[(methylamino)methyl]-1H-benzimidazol-2-yl}-1,3-thiazol-2-amine hydrochloride:

| Example | | Name | Data |
|---|---|---|---|
| 180 | 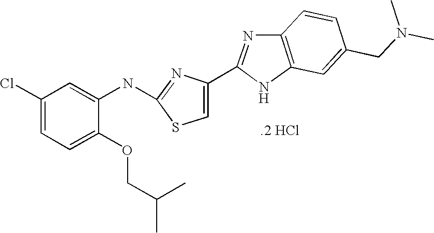 | N-{5-Chloro-2-[(2-methylpropyl)oxy]phenyl}-4-{5-[(dimethylamino)methyl]-1H-benzimidazol-2-yl}-1,3-thiazol-2-aminehydrochloride | LC/MS Rt =2.27, [MH+]456, 458 |
| 181 | 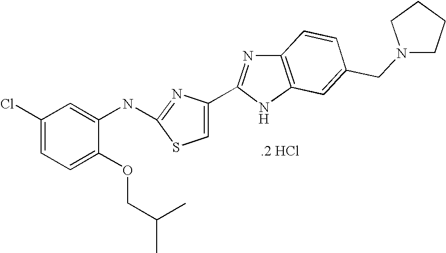 | N-{5-Chloro-2-[(2-methylpropyl)oxy]phenyl)-4-[5-(1-pyrrolidinylmethyl)-1H-benzimidazol-2-yl]-1,3-thiazol-2-amine | LC/MS Rt =2.34, [MH+]482, 484 |
| 182 | 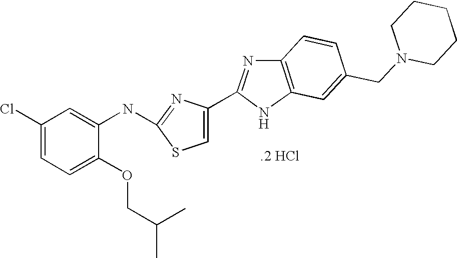 | N-{5-Chloro-2-[(2-methylpropyl)oxy]phenyl}-4-[5-(1-piperidinylmethyl)-1H-benzimidazol-2-yl]-1,3-thiazol-2-amine | LC/MS Rt =2.41, [MH+]496, 498 |
| 183 | 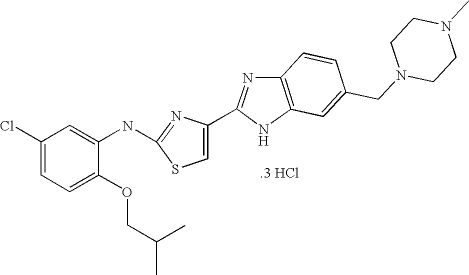 | N-{5-Chloro-2-[(2-methylpropyl)oxy]phenyl}-4-{5-[(4-methyl-1-piperazinyl)methyl]-1H-benzimidazol-2-yl}-1,3-thiazol-2-amine | LC/MS Rt =2.16, [MH+]511, 513 |

It is to be understood that the present invention covers all combinations of particular and preferred subgroups described herein above.

It is to be understood that the present invention covers all combinations of particular and preferred subgroups described herein above.

### Assays for Determining Biological Activity

The compounds of formula (I) can be tested using the following assays to demonstrate their prostanoid antagonist or agonist activity in vitro and in vivo and their selectivity. Prostaglandin receptors that may be investigated are DP, EP₁, EP₂, EP₃, EP₄, FP, IP and TP.

### Biological Activity at EP₁ and EP3 Receptors

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable (pCIN; BioTechniques 20 (1996): 102-110) vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 g/ml puromycin. For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds. The data so generated may be analysed by means of a computerised curve-fitting routine. The concentration of compound that elicits a half-maximal inhibition of the calcium mobilisation induced by PGE₂ (pIC₅₀) may then be estimated.

### Binding Assay for the Human Prostanoid EP₁ Receptor

Competition assay using [³H]-PGE2.

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10 s burst at full setting), centrifuged at 48,000×g for 20mins and the pellet containing the membrane fraction is washed (optional) three times by suspension and centrifugation at 48,000×g for 20mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at 80° C. until required.

For the binding assay the cell membranes, competing compounds and [³H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques to determine the concentration of compound producing 50% inhibition of specific binding (IC₅₀).

### Biological Activity at TP Receptor

To determine if a compound has agonist or antagonist activity at the TP receptor a functional calcium mobilisation assay may be performed. Briefly, the antagonist properties of compounds are assessed by their ability to inhibit the mobilisation of intracellular calcium ([Ca²⁺]ᵢ) in response to activation of TP receptors by the stable TXA₂ mimetic U46619 (9,11-dideoxy-11α,9α-epoxy-methanoprostaglandin F2α; commercially available from e.g Sigma-Aldrich). Increasing concentrations of antagonist reduce the amount of calcium that a given concentration of U46619 can mobilise. The net effect is to displace the U46619 concentration-effect curve. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software. The agonist activity of the compounds are determined by their ability to cause an increase in intracellular mobilisation in the absence of U46619.

The human TP calcium mobilisation assay utilises Chinese hamster ovary-K1 (CHO-K1) cells into which a stable (pCIN; BioTechniques 20 (1996): 102-110) vector containing TP cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 96-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of U46619 are then added to the plate in order to assess the antagonist properties of the compounds.

The data so generated may be analysed by means of a computerised curve-fitting routine. The concentration of compound that elicits a half-maximal inhibition of the calcium mobilisation induced by U46619 (pIC₅₀) may then be estimated, and the percentage activation caused by the compounds directly can be used to determine if there is any agonism present.

### Results

The compounds of examples 1-183 were tested in the binding assay for the human prostanoid EP₁ receptor. The results are expressed as pIC₅ values. A pIC₅₀ is the negative logarithms of the IC₅₀. The results given are averages of a number of experiments. The compounds of examples 1-6, 8-160 and 162-183 had a pIC₅₀ value ≧6. More particularly, the compounds of examples 9-18, 22-24, 26, 27, 29-34, 36, 37, 39, 41, 43-45, 51-54, 56-61, 65, 71, 73, 74, 76-78, 83-87, 89,100-109, 115-117, 122-126, 132, 135, 136, 138, 140, 142, 146,148-154, 162-169, 174-180 and 183 exhibited a pIC₅₀ value ≧7.5. The compounds of examples 7 and 161 exhibited pIC₅₀ values of <6.

The compounds of examples 46-49, 50, 79-81, 91-99, 101-108, 110-121, 124-37, 139, 140-155, 158-160, and 164-183 (free bases or sodium salts) were tested in the human EP₁ calcium mobilisation assay. The results are expressed as functional pKᵢ values. A functional pKᵢ is the negative logarithms of the antagonist dissociation constant as determined in the human EP₁ calcium mobilisation assay. The results given are averages of a number of experiments. The compounds of examples 46-49, 50, 94-96, 98, 101-108, 111, 113-117, 124-126, 129-137, 139, 140, 142, 144-155, 158-160, 164, 166-178, and 183 exhibited a functional pKi value >6. More particularly, the compounds of examples 98, 106, 108, 135, 136, 144 and 154 exhibited a functional pKi value of ≧7.5. The compounds of examples 79-81, 91-93, 97, 99, 110, 112, 118-121, 127, 128, 141, 143, 165, and 179-182 exhibited a functional pKi value <6.

The compounds of examples 46-50, 79-82, 90-99, 101-137, 139-160, and 166-183 (free bases or sodium salts) were tested in the human EP₃ calcium mobilisation assay. The results are expressed as functional pKᵢ values. A functional pKi is the negative logarithms of the antagonist dissociation constant as determined in the human EP₃ calcium mobilisation assay. The results given are averages of a number of experiments. The compounds of examples 46, 47, 49, 50, 79-82, 90-93, 95-99, 101,104-108, 110-137, 139-160, and 166-183 exhibited a functional pKi value of <6.5. The compounds of examples 50, 79-82, 91-93, 95, 97, 99, 104, 107, 108, 110, 112-126, 129-131, 133, 136, 139-147, 149, 151-154, 156, 157, 159, 160, and 166-183 showed no activity in a functional assay.

No toxicological effects were observed in these tests.

The application of which this description and claims forms part may be used as a basis for priority in respect of any subsequent application. The claims of such subsequent application may be directed to any feature or combination of features described herein.

They may take the form of product, composition, process, or use claims and may include, by way of example and without limitation the following claims:

What is claimed is:

1. A compounds of formula (I): 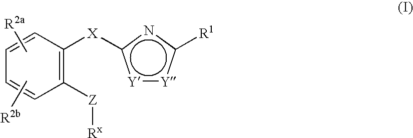
wherein:
either Y′ is CH and Y″ is O or S, or Y′ is O or S and Y″ is CH thus forming an oxazole or a thiazole ring;
X is CR⁷R⁸, O, NR⁴, S, SO, or SO₂, or X is a bond;
Z is O, S, SO or SO₂;
R^{x} is optionally substituted C₃₋₁₀alkyl, optionally substituted C₃₋₁₀alkenyl, optionally substituted C₃₋₁₀alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;
R¹ is CO₂H, CQ^{c}Q^{d}CO₂H, tetrazolyl, CH₂tetrazolyl, CONR⁴R⁵, NR⁴CO₂R⁶, NR⁴COR⁶ or 1,2,4-triazol-3-yl optionally substituted on a ring carbon; or R¹ represents imidazolyl or pyrazolyl wherein optionally the imidazole or pyrazole ring is fused to give an optionally substituted bicyclic or tricyclic ring system;
R^{2a} and R^{2b} independently represents hydrogen, halo, CN, SO₂alkyl, SR⁴ or NO₂; or optionally substituted alkyl or optionally substituted alkoxy;
R⁴ is hydrogen or optionally substituted alkyl;
R⁵ is hydrogen or optionally substituted alkyl, optionally substituted aryl, optionally substituted heterocyclyl, optionally substituted SO₂aryl, optionally substituted SO₂alkyl, optionally substituted SO₂heterocyclyl, optionally substituted CQ^{a}Q^{b}aryl, or optionally substituted CQ^{a}Q^{b} heterocyclyl; or
R⁴ and R⁵ together with the nitrogen to which they are attached form a heterocyclic or bicyclic heterocyclic ring;
R⁶ is optionally substituted alkyl or optionally substituted aryl;
R⁷ is hydrogen, fluorine or alkyl;
R⁸ is hydrogen, hydroxy, fluorine or alkyl;
or R⁷ and R⁸ together with the carbon to which they are attached form a cycloalkyl ring, optionally containing up to one heteroatom selected from O, S, NH and N-alkyl; or R⁷ and R⁸ together with the carbon to which they are attached form a carbonyl group; and
Q^{a} and Q^{b} are each independently selected from hydrogen, CH₃ and fluorine;
Q^{c} and Q^{d} are each independently selected from hydrogen and CH₃;
or a derivative thereof;
provided that:
when X is a bond, then R¹ is CQ^{c}Q^{d}CO₂H;
when X is CR⁷R⁸, then R¹ is not CQ^{c}Q^{d} CO₂H;
when R¹ is benzimidazolyl it is unsubstituted on the 1-position; and
when R¹ is benzimidazole optional substituents on the 4 or 7 position are selected from CH₂OH or CO₂H.

2. A compound according to claim 1 which is a compound of formula (IA): 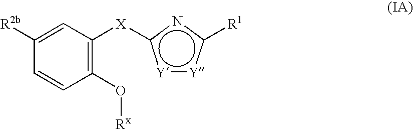
wherein:
either Y′ is CH and Y″ is O or S, or Y′ is O or S and Y″ is CH thus forming an oxazole or a thiazole ring;
X is CR⁷R⁸, or NR⁴, or X is a bond;
R^{x} is optionally substituted C₃₋₁₀alkyl, optionally substituted C₃₋₁₀alkenyl, optionally substituted C₃₋₁₀alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;
R¹ is CO₂H, CQ^{c}Q^{d}CO₂H, tetrazolyl, CH₂tetrazolyl, CONR⁴R⁵, NR⁴COR⁶ or 1,2,4-triazol-3-yl optionally substituted on a ring carbon; or R¹ represents imidazolyl or pyrazolyl wherein optionally the imidazole or pyrazole ring is fused to give an optionally substituted bicyclic or tricyclic ring system;
R^{2b} is Cl, Br, or CF₃.
R⁴ is hydrogen or optionally substituted alkyl;
R⁵ is hydrogen or optionally substituted alkyl, optionally substituted aryl, optionally substituted heterocyclyl, optionally substituted SO₂aryl, optionally substituted SO₂alkyl, optionally substituted SO₂heterocyclyl, optionally substituted CQ^{a}Q^{b}aryl, or optionally substituted CQ^{a}Q^{b} heterocyclyl; or
R⁴ and R⁵ together with the nitrogen to which they are attached form a heterocyclic or bicyclic heterocyclic ring;
R⁶ is optionally substituted alkyl or optionally substituted aryl;
R⁷ is hydrogen, fluorine or alkyl;
R⁸ is hydrogen, hydroxy, fluorine or alkyl;
or R⁷ and R⁸ together with the carbon to which they are attached form a cycloalkyl ring, optionally containing up to one heteroatom selected from O, S, NH and N-alkyl; or R⁷ and R⁸ together with the carbon to which they are attached form a carbonyl group; and
Q^{a} and Q^{b} are each independently selected from hydrogen, CH₃ and fluorine;
Q^{c} and Q^{d} are each independently selected from hydrogen and CH₃;
or a derivative thereof;
provided that:
when X is a bond, then R¹ is CQ^{c}Q^{d} CO₂H;
when X is CR⁷R⁸, then R¹ is not CQ^{c}Q^{d}CO₂H;
when Y′ or Y″ is O, then R¹ is not CQ^{c}Q^{d} CO₂H;
when R¹ is benzimidazolyl it is unsubstituted on the 1-position; and
when R¹ is benzimidazole optional substituents on the 4 or 7 position are selected from CH₂OH or CO₂H.

3. (canceled)

4. A pharmaceutical composition comprising a compound according to claim 1 or a pharmaceutically acceptable derivative thereof together with a pharmaceutical carrier and/or excipient.

5. A compound according to claim 1 or a pharmaceutically acceptable derivative thereof for use as an active therapeutic substance.

6. (canceled)

7. (canceled)

8. A method of treating a human or animal subject suffering from a pain, or an inflammatory, immunological, bone, neurodegenerative or renal disorder, which method comprises administering to said subject an effective amount of a compound according to claim 1 or a pharmaceutically acceptable derivative thereof.

9. A method of treating a human or animal subject suffering from inflammatory pain, neuropathic pain or visceral pain which method comprises administering to said subject an effective amount of a compound according to claim 1 or a pharmaceutically acceptable derivative thereof.

10. (canceled)

11. (canceled)

12. (canceled)
